(19) United States
(12) Patent Application Publication
(10) Pub. No.: US 2026/0001862 A1
(43) Pub. Date: Jan. 1, 2026

(54) QUINOXALINEDIONE AND PYRIDO[2,3-B]PYRAZINE-2,3-DIONE B CELL LYMPHOMA 6 (BCL6) DEGRADERS AND USES THEREOF

(71) Applicant: DANA-FARBER CANCER INSTITUTE, INC., Boston, MA (US)

(72) Inventors: Lyn Howard JONES, Boston, MA (US); Jianwei CHE, Boston, MA (US); Huang HUANG, Boston, MA (US); Nikki KONG, Boston, MA (US); Justin CRUITE, Boston, MA (US); Yingpeng LIU, Boston, MA (US)

(73) Assignee: DANA-FARBER CANCER INSTITUTE, INC., Boston, MA (US)

(21) Appl. No.: 18/993,089

(22) PCT Filed: Jul. 18, 2023
(86) PCT No.: PCT/US2023/027970

§ 371 Date: Jan. 10, 2025

Related U.S. Application Data
(60) Provisional application No. 63/390,482, filed on Jul. 19, 2022.

(51) Int. Cl. C07D 401/14; A61K 31/498; A61K 31/506; A61K 45/06; C07D 405/14; C07D 409/14; C07D 413/14; C07D 471/04

(52) U.S. Cl. CPC C07D 401/14; A61K 31/498; A61K 31/506; A61K 45/06; C07D 405/14; C07D 409/14; C07D 413/14; C07D 471/04

(57) ABSTRACT

Described are the compounds, compositions and methods of treating a cancer characterized by aberrant B-cell lymphoma 6 (BCL6) activity.

### RELATED APPLICATIONS

This application claims the benefit of priority under 35 U.S.C. § 119(e) to U.S. Provisional Application No. 63/390,482, filed Jul. 19, 2022, which is incorporated herein by reference in its entirety.

### BACKGROUND

BCL6 is a promising drug target for non-Hodgkin lymphomas such as diffuse large B cell lymphoma (DLBCL) (Cerchietti et al., Cancer Cell 17:400-411 (2010); Cardenas et al., J. Clin. Invest. 126:3351-3362 (2016)) and follicular lymphoma (Bosga-Bouwer et al., Genes Chromosomes Cancer 44:301-304 (2005)). Pathologically increased BCL6 expression, as a result of somatic BCL6 translocation, exonic mutation, promoter mutation, or mutations in regulatory pathways, is a common driver of B cell malignancies (Hatzi et al., Trends Mol. Med. 20:343-352 (2014)). In genetically engineered mice, overexpression of BCL6 drives lymphoma development (Cattoretti et al., Cancer Cell 7:445-455 (2005)). BCL6 acts as a master transcriptional repressor enabling rapid expression of germinal center (GC) B cells and tolerance to genomic instability caused by hypermutation of the immunoglobulin genes and class switch recombination (Hatzi et al., Trends Mol. Med. 20:343-352 (2014)). BCL6 represses a broad range of genes involved in the DNA damage response (Ranuncolo et al., Blood Cells Mol. Dis. 41:95-99 (2008)), cell cycle checkpoints (Tunyaplin et al., J. Immunol. 173:1158-1165 (2004)), and differentiation (Phan et al., Nat. Immunol. 6:1054-1060 (2005)).

Knock-out of BCL6 in lymphoma cells results in tumor stasis (Schlager et al., Oncotarget 11:875-890 (2020)). Several peptide and small molecule inhibitors targeting BCL6 have shown efficacy in vivo, but only at high concentrations, which has limited their translation into clinical therapeutic agents (Cerchietti et al., Cancer Cell 17:400-411 (2010); Cardenas et al., J. Clin. Invest. 126:3351-3362 (2016)).

### SUMMARY

A first aspect of the present disclosure is directed to a compound having a structure represented by formula I:

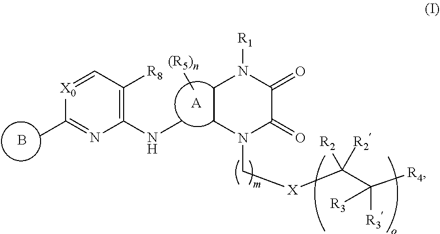

or a pharmaceutically acceptable salt or stereoisomer thereof, wherein:

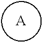

is phenyl or pyridyl;

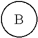

is

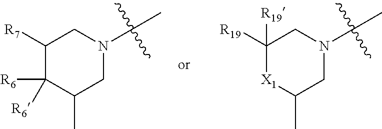

X is a bond, (C₃-C₆) carbocyclyl, (C₃-C₆) carbocyclyl(C═O), or SO₂;
X₀ is N or CCN,
X₁ is CH₂, S, CHF, CHCl, CHOH, or CF₂;
R₁ is (C₁-C₆) alkyl, (C₁-C₆) hydroxyalkyl, (C₁-C₆) aminoalkyl, (C₃-C₆) carbocyclyl, 4- to 6-membered heterocyclyl, (C₁-C₆) alkyl-(C₃-C₆) carbocyclyl, or (C₁-C₆) alkyl-4- to 6-membered heterocyclyl, wherein said alkyl, hydroxyalkyl, aminoalkyl, carbocyclyl, or heterocyclyl is further optionally substituted by one or more, identical or different R₁ₐ groups, wherein R₁ₐ is deuterium, (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl;
R₂ and R_{2′} are each independently H, (C₁-C₃) alkyl, (C₁-C₃) hydroxyalkyl, or (C₁-C₃) aminoalkyl,
or R₂ and R_{2′}, together with the same carbon atom to which they are attached, form (C₃-C₆) carbocyclyl or 4- to 6-membered heterocyclyl, wherein said alkyl, hydroxyalkyl, aminoalkyl, carbocyclyl, or heterocyclyl is optionally substituted by one or more, identical or different R₂ₐ groups, wherein R₂ₐ is (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl;
R₃ and R_{3′} are each independently H, (C₁-C₃) alkyl, (C₁-C₃) hydroxyalkyl, or (C₁-C₃) aminoalkyl, or
R₃ and R_{3′}, together with the same carbon atom to which they are attached, form C═O, (C₃-C₆) carbocyclyl, 4- to 6-membered heterocyclyl, wherein said carbocyclyl or heterocyclyl is optionally substituted by one or more, identical or different R₃ₐ groups, wherein each R₃ₐ is independently (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl, or wherein two R₃ₐ groups, together with the same carbon atom to which they are attached, form C═O,
or wherein R₂ or R_{2′} and R₃ or R_{3′}, together with the same carbon atom to which they are attached, form (C₃-C₆) carbocyclyl or 4- to 6-membered heterocyclyl, wherein said carbocyclyl or heterocyclyl is optionally substituted by one or more, identical or different R₂ₐ groups;
R₄ is H, OH, NH—(C₁-C₆) alkyl, NH—(C₁-C₆) hydroxyalkyl, NH—(C₁-C₆) aminoalkyl, NH—(C₃-C₆) carbocyclyl, NH-4- to 6-membered heterocyclyl, NH—(C₁-C₆) alkyl-(C₃-C₆) carbocyclyl, NH—(C₁-C₆) alkyl-4- to 6-membered heterocyclyl, wherein said alkyl, hydroxyalkyl, aminoalkyl, carbocyclyl, or heterocyclyl is further optionally substituted by one or more, identical or different R₄ₐ groups, wherein each R₄ₐ is independently (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl;
R₅ is halogen or (C₁-C₃) alkoxy;
R₆ and R₆, are each independently H or halogen;
R₇ is H, halogen, (C₁-C₄) alkyl, (C₁-C₄) alkoxy, (C₁-C₄) haloalkyl, (C₁-C₄) haloalkoxy, CN, NO₂, OH, (C₂-C₄) alkenyl, (C₂-C₄) alkynyl, or —R₇ₐ-R_{7b}, wherein R₇ₐ is absent or O, N(R₇ₐ₁)(CR₇ₐ₁R₇ₐ₂)_{q}, S, SO, SO₂, C(O), C(O)O, OC(O), C(O)N(R₇ₐ₁), N(R₇ₐ₁)C(O), N(R₇ₐ₁)C(O)N(R₇ₐ₂), N(R₇ₐ₃)C(O)O, OC(O)N(R₇ₐ₁), S(O)₂N(R₇ₐ₁), N(R₇ₐ₁)SO₂, wherein R₇ₐ₁ and R₇ₐ₂ are each independently H or (C₁-C₄) alkyl, and q is 0, 1, 2, and R_{7b} is H, (C₁-C₆) alkyl, (C₆-C₁₀) aryl, (C₃-C₆) cycloalkyl, (C₂-C₄) alkenyl, (C₁-C₄) alkynyl, (C₃-C₆) cycloalkenyl, wherein said alkyl, aryl, cycloalkyl, alkenyl, alkynyl, or cycloalkenyl is optionally further substituted by one or more substituent groups independently selected from oxo, (C₁-C₄) alkyl, (C₁-C₄) cycloalkyl, halogen, (C₁-C₄) haloalkyl, (C₁-C₄) haloalkoxy, (C₁-C₄) hydoxyalkyl, amino, CN, OH, amido, carboxy, carbamoyl, sulphamoyl, mercapto, C(O)NR_{7b1}R_{7b2}, R_{7b1}R_{7b2}, or OR_{7b1}, wherein R_{7b1} and R_{7b2} each is independently selected from hydrogen, (C₁-C₄) alkyl, (C₃-C₆) cycloalkyl, wherein said oxo, alkyl, cycloalkyl, haloalkyl, haloalkoxy, hydroxyalkyl, amino, CN OH, amido, carboxy, carbamoyl, sulphamoyl, or mercapto is optionally further substituted by R_{7b3}—R_{7b4}, wherein R_{7b3} is absent or (C₁-C₅) alkylene optionally substituted by one or more substituents selected from (C₁-C₂) alkyl or oxo, and R_{7b4} is (C₆-C₁₀) aryl, 5- to 12-membered heteroaryl, 4- to 12-membered heterocyclyl, (C₃-C₆) carbocyclyl, halogen, (C₁-C₄) haloalkyl, (C₁-C₄) haloalkoxy, CN, OH, (C₁-C₄) alkoxy, C(O)R_{7b4a}, COOR_{7b4b}, C(O)NR_{7b4a}R_{7b4b}, or NR_{7b4a}R_{7b4b}, wherein: R_{7b4a} and R_{7b4b} are each independently selected from H or (C₁-C₄) alkyl; said aryl, heteroaryl, heterocyclyl, or carbocyclyl is optionally further substituted by one or more substituent groups independently selected from (C₁-C₄)alkyl, halogen, (C₁-C₄) haloalkyl, amino, CN or OH; and said heterocyclyl or heteroaryl contains at least one nitrogen atom and is linked via nitrogen, wherein said heterocyclyl group is optionally and independently substituted by one or more, identical or different groups independently selected from R₉, and said heteroaryl group is optionally and independently substituted by one or more, identical or different group independently selected from R₁₀;
R₈ is halogen or CN;
R₉ is ═O, CN, C≡CH, OH, COOH, halogen, (C₁-C₆) alkyl, (C₁-C₆) alkoxy, (C₁-C₆) haloalkyl, 5 or 6 membered heteroaryl, phenyl, N(R₁₁R₁₂), C(O)—R₁₃, C(O)N(R₁₄R₁₅), or 5- to 8-membered heterocyclyl, wherein said (C₁-C₆) alkyl is optionally substituted with COOH, OH, COO—(C₁-C₆) alkyl, —CON((C₁-C₆) alkyl)₂, (C₁-C₆) alkoxy, N((C₁-C₃) alkyl)₂, phenyl, or 5- or 6-membered heterocyclyl, and said heteroaryl, phenyl, or heterocyclyl is optionally substituted with one group selected from (C₁-C₆) alkyl;
R₁₀ is COOH, (C₁-C₆) alkyl, C(O)—R₁₆, or C(O)N(R₁₇)(R₁₈);
R₁₁ is H or (C₁-C₄) alkyl;
R₁₂ is H, (C₁-C₄) alkyl, (C₁-C₄) haloalkyl, 6-membered heterocyclyl, or 6-membered heteroaryl;
R₁₃ is (C₁-C₃) alkyl-N((C₁-C₃) alkyl)₂ or 5- or 6-membered heterocyclyl, wherein said heterocyclyl is optionally substituted with (C₁-C₃) alkyl;
R₁₄ is hydrogen or (C₁-C₃) alkyl;
R₁₅ is (C₁-C₆) alkyl optionally substituted with NH₂, (C₁-C₆) alkoxy, O—(C₁-C₆) alkyl-NH₂, or O—(C₁-C₆) alkyl-O—(C₁-C₆) alkyl-NH₂, or R₁₅ is a 6-membered heterocyclyl optionally substituted with (C₁-C₃) alkyl;
R₁₆ is a 6-membered heterocyclyl optionally substituted with (C₁-C₃) alkyl;
R₁₇ and R₁₈ are each independently H or (C₁-C₃) alkyl;
R₁₉ is H, ═O, —CN, —C≡CH, —OH, —SH, —NH₂, —COOH, halo, (C₁-C₆)alkyl, —O—(C₁-C₆)alkyl, (C₁-C₆)haloalkyl, amido, carboxy, carbamoyl, sulfamoyl, phenyl, 5- to 8-membered heterocyclyl, —NR₂₀R₂₁, —C(O)R₂₂, —C(O)NR₂₃R₂₄, or L₁Y₁, wherein said alkyl, phenyl, or heterocyclyl is optionally substituted with one or more groups selected from halo, —COOH, —OH, —NH₂, (C₁-C₆)alkyl, —C(O)O—(C₁-C₆)alkyl, —C(O)N(C₁-C₆ alkyl)₂, —O—(C₁-C₆)alkyl, —N(C₁-C₃ alkyl)₂, phenyl, and 4- to 6-membered heterocyclyl, optionally substituted with one or more groups selected from halo and (C₁-C₆)alkyl,
L₁ is absent, (C₁-C₆)alkylene or (C₃-C₇)carbocyclyl; wherein said alkylene or carbocyclyl is further optionally substituted by one or more, identical or different R₂₅ groups, or L₁ is (C₂-C₄)alkylene which is bound to R₂₆ to form a 4- to 6-membered heterocyclyl group; and
Y₁ is —CN, —OH, halo, (C₁-C₄)alkoxy, (C₁-C₄)haloalkyl, (C₁-C₄)haloalkoxy, 4- to 7-membered heterocyclyl, (C₃-C₆)carbocyclyl, —NR₂₆R₂₇, —C(O)R₂₂, —C(O)NR₂₃R₂₄, wherein said alkyl, carbocyclyl, or heterocyclyl is further optionally substituted by one or more, identical or different groups selected from (C₁-C₄)alkyl, halo, (C₁-C₄)haloalkyl, —CN, —OH, and —NH₂;
R₁₉′ is absent, H, —CN, —C≡CH, —OH, —SH, —NH₂, —COOH, halo, (C₁-C₆)alkyl, —O—(C₁-C₆)alkyl, (C₁-C₆)haloalkyl, amido, carboxy, carbamoyl, sulfamoyl, phenyl, 5- to 8-membered heterocyclyl, —NR₂₀R₂₁, —C(O)R₂₂, or —C(O)NR₂₃R₂₄; wherein said alkyl, phenyl, or heterocyclyl is further optionally substituted by one or more, identical or different R₂₅ groups, or
R₁₉′ and L₁ together with the same carbon atom to which they are attached form a spiro (C₃-C₇)carbocyclyl group or a 4- to 7-membered heterocyclyl group; wherein said carbocyclyl or heterocyclyl is further optionally substituted by one or more, identical or different R₂₅ groups;
R₂₀ is hydrogen, (C₁-C₄)alkyl, or (C₃-C₆)cycloalkyl;
R₂₁ is hydrogen, (C₁-C₄)alkyl, (C₁-C₄)haloalkyl, (C₃-C₆)cycloalkyl, or 6-membered heterocyclyl;
R₂₂ is —(C₁-C₃)alkyl-N(C₁-C₃ alkyl)₂, (C₃-C₆)cycloalkyl, or 5- to 6-membered heterocyclyl, wherein said heterocyclyl is optionally substituted with (C₁-C₃)alkyl;
R₂₃ is hydrogen, (C₁-C₃)alkyl, or (C₃-C₆)cycloalkyl;
R₂₄ is (C₃-C₆)cycloalkyl or (C₁-C₆)alkyl optionally substituted with —NH₂, —O—(C₁-C₆)alkyl, —O—(C₁-C₆)alkyl-NH₂, or —O—(C₁-C₆)alkyl-O—(C₁-C₆)alkyl-NH₂;
each R₂₅ is independently oxo, alkyl, alkenyl, alkynyl, halo, haloalkyl, carbocyclyl, heterocyclyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, or phosphinyl;
R₂₆ and R₂₇ are each independently hydrogen, (C₁-C₆)alkyl, (C₃-C₇)carbocyclyl, 4- to 7-membered heterocyclyl, (C₆-C₁₀)aryl, or monocyclic or bicyclic 5- to 10-membered heteroaryl;
wherein said alkyl, carbocyclyl, heterocyclyl, aryl or heteroaryl is further optionally substituted by one or more, identical or different R₂₅ groups, or
R₂₆ and R₂₇ together with the nitrogen atom to which they are attached form a 3- to 7-membered heterocyclyl, wherein said heterocyclyl is further optionally substituted by one or more, identical or different R₂₅ groups;
m is 0, 1, or 2;
n is 0, 1, 2, or 3; and
o is 0 or 1.

Another aspect of the present disclosure is directed to a pharmaceutical composition that includes a therapeutically effective amount of a compound of formula (I) or a pharmaceutically acceptable salt or stereoisomer thereof, and a pharmaceutically acceptable carrier.

A further aspect of the present disclosure is directed to a method of treating cancer that is characterized or mediated by aberrant B cell lymphoma 6 (BCL6) activity that entails administering to a subject in need thereof a therapeutically effective amount of a compound of formula (I) or a pharmaceutically acceptable salt or stereoisomer thereof.

In some embodiments, the cancer is a lymphoid malignancy. In some embodiments, the lymphoid malignancy is peripheral T-cell lymphoma (PTCL), diffuse large B-cell lymphoma (DLBCL), mantle cell lymphoma (MCL), follicular lymphoma (FL), chronic lymphocytic leukemia (CLL), acute lymphoblastic leukemia/lymphoma (ALL), cutaneous T-cell lymphoma, chronic myeloid leukemia, or B-cell non-Hodgkin's lymphoma.

### DETAILED DESCRIPTION

Unless defined otherwise, all technical and scientific terms used herein have the same meaning as is commonly understood by one of skill in the art to which the subject matter herein belongs. As used in the specification and the appended claims, unless specified to the contrary, the following terms have the meaning indicated in order to facilitate the understanding of the present disclosure.

As used in the description and the appended claims, the singular forms “a”, “an”, and “the” include plural referents unless the context clearly dictates otherwise. Thus, for example, reference to “a composition” includes mixtures of two or more such compositions, reference to “an inhibitor” includes mixtures of two or more such inhibitors, and the like.

Unless stated otherwise, the term “about” means within 10% (e.g., within 5%, 2% or 1%) of the particular value modified by the term “about.”

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

With respect to compounds of the present disclosure, and to the extent the following terms are used herein to further describe them, the following definitions apply.

As used herein, the term “alkyl” refers to a saturated linear or branched-chain monovalent hydrocarbon radical. In some embodiments, and to the extent not specified otherwise with respect to any one or more groups in the compounds of formula (I), the alkyl radical is a C₁-C₄ group. In other embodiments, the alkyl radical is a C₀-C₄, C₀-C₃, C₁-C₄, C₁-C₃ or C₁-C₂ group (wherein C₀ alkyl refers to a bond). Representative examples of alkyl groups include methyl, ethyl, 1-propyl, 2-propyl, i-propyl, 1-butyl, 2-methyl-1-propyl, 2-butyl, and 2-methyl-2-propyl. In some embodiments, an alkyl group is a C₁-C₃ alkyl group. In some embodiments, an alkyl group is a C₃-C₅ branched-chain alkyl group.

As used herein, the term “alkylene” refers to a straight or branched divalent hydrocarbon chain linking the rest of the molecule to a radical group, consisting solely of carbon and hydrogen. The alkylene chain may be attached to the rest of the molecule through a single bond and to the radical group through a single bond. In some embodiments, and to the extent not specified otherwise with respect to any one or more groups in the compounds of formula (I), the alkylene group contains one to 4 carbon atoms (C₁-C₄ alkylene (e, methylene, ethylene, propylene, and n-butylene)). In other embodiments, an alkylene group contains one to 3 carbon atoms (C₁-C₃ alkylene). In other embodiments, an alkylene group contains one to 2 carbon atoms (C₁-C₂ alkylene). In other embodiments, an alkylene group contains one carbon atom (C₁ alkylene).

As used herein, the term “alkenyl” refers to a linear or branched-chain monovalent hydrocarbon radical with at least one carbon-carbon double bond. An alkenyl includes radicals having “cis” and “trans” orientations, or alternatively, “E” and “Z” orientations. In some embodiments, and to the extent not specified otherwise with respect to any one or more groups in the compounds of formula (I), the alkenyl radical is a C₂-C₆ group. In other embodiments, the alkenyl radical is a C₂-C₆ or C₂-C₃ group. Examples include ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl and hexa-1,3-dienyl.

The terms “alkoxyl” or “alkoxy” as used herein refer to an alkyl group, as defined above, having an oxygen radical attached thereto. Representative alkoxyl groups include methoxy, ethoxy, propyloxy, tert-butoxy and the like. An “ether” is two hydrocarbyl groups covalently linked by an oxygen. Accordingly, the substituent of an alkyl that renders that alkyl an ether is or resembles an alkoxyl, such as can be represented by one of —O-alkyl, —O-alkenyl, and —O-alkynyl.

As used herein, the term “alkoxylene” refers to a saturated monovalent aliphatic radicals of the general formula (—O—CₙH₂ₙ—) where n represents an integer (e.g., 1, 2, 3, 4, 5, 6, or 7) and is inclusive of both straight-chain and branched-chain radicals. The alkoxylene chain may be attached to the rest of the molecule through a single bond and to the radical group through a single bond. In some embodiments, and to the extent not specified otherwise with respect to any one or more groups in the compounds of formula (I), the alkoxylene group contains one to 3 carbon atoms (—O—C₁-C₃ alkoxylene). In other embodiments, an alkoxylene group contains one to 5 carbon atoms (—O—C₁-C₅ alkoxylene).

As used herein, the term “cyclic group” broadly refers to any group that used alone or as part of a larger moiety, contains a saturated, partially saturated or aromatic ring system e.g., carbocyclic (cycloalkyl, cycloalkenyl), heterocyclic (heterocycloalkyl, heterocycloalkenyl), aryl and heteroaryl groups. Cyclic groups may have one or more (e.g., fused) ring systems. Thus, for example, to the extent not specified otherwise with respect to any one or more groups in the compounds of formula (I), a cyclic group can contain one or more (e.g., 1, 2, or 3) carbocyclic, heterocyclic, aryl or heteroaryl groups.

As used herein, the term “carbocyclic” (also “carbocyclyl”) refers to a group that used alone or as part of a larger moiety, contains a saturated, partially unsaturated, or aromatic ring system having 3 to 20 carbon atoms, that is alone or part of a larger moiety (e.g., an alkcarbocyclic group). The term carbocyclyl includes mono-, bi-, tri-, fused, bridged, and spiro-ring systems, and combinations thereof. To the extent not specified otherwise with respect to any one or more groups in the compounds of formula (I), in one embodiment, carbocyclyl includes 3 to 15 carbon atoms (C₃-C₁₅). In one embodiment, carbocyclyl includes 3 to 12 carbon atoms (C₃-C₁₂). In another embodiment, carbocyclyl includes C₃-C₈, C₃-C₁₀ or C₅-C₁₀. In another embodiment, carbocyclyl, as a monocycle, includes C₃-C₈, C₃-C₆ or C₅-C₆. In some embodiments, carbocyclyl, as a bicycle, includes C₇-C₁₂. In another embodiment, carbocyclyl, as a spiro system, includes C₅-C₁₂. Representative examples of monocyclic carbocyclyls include cyclopropyl, cyclobutyl, cyclopentyl, 1-cyclopent-1-enyl, 1-cyclopent-2-enyl, 1-cyclopent-3-enyl, cyclohexyl, perdeuteriocyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, phenyl, and cyclododecyl; bicyclic carbocyclyls having 7 to 12 ring atoms include [4,3], [4,4], [4,5], [5,5], [5,6] or [6,6] ring systems, such as for example bicyclo[2.2.1]heptane, bicyclo[2.2.2]octane, naphthalene, and bicyclo[3.2.2]nonane. Representative examples of spiro carbocyclyls include spiro[2.2]pentane, spiro[2.3]hexane, spiro[2.4]heptane, spiro[2.5]octane and spiro[4.5]decane. The term carbocyclyl includes aryl ring systems as defined herein. The term carbocyclyl also includes cycloalkyl rings (e.g., saturated or partially unsaturated mono-, bi-, or spiro-carbocycles). The term carbocyclic group also includes a carbocyclic ring fused to one or more (e.g., 1, 2 or 3) different cyclic groups (e.g., aryl or heterocyclic rings), where the radical or point of attachment is on the carbocyclic ring.

As used herein, the term “heterocyclyl” refers to a “carbocyclyl” that used alone or as part of a larger moiety, contains a saturated, partially unsaturated or aromatic ring system, wherein one or more (e.g., 1, 2, 3, or 4) carbon atoms have been replaced with a heteroatom (e.g., O, N, N(O), S, S(O), or S(O)₂). The term heterocyclyl includes mono-, bi-, tri-, fused, bridged, and spiro ring systems, and combinations thereof. In some embodiments, to the extent not specified otherwise with respect to any one or more groups in the compounds of formula (I), a heterocyclyl refers to a 3 to 15 membered heterocyclyl ring system. In some embodiments, a heterocyclyl refers to a 3 to 12 membered heterocyclyl ring system. In some embodiments, a heterocyclyl refers to a saturated ring system, such as a 3 to 12 membered saturated heterocyclyl ring system. In some embodiments, a heterocyclyl refers to a heteroaryl ring system, such as a 5 to 14 membered heteroaryl ring system. The term heterocyclyl also includes C₃-C₈ heterocycloalkyl, which is a saturated or partially unsaturated mono-, bi-, or spiro-ring system containing 3-8 carbons and one or more (1, 2, 3 or 4) heteroatoms.

In some embodiments, a heterocyclyl group includes 3-12 ring atoms and includes monocycles, bicycles, tricycles and spiro ring systems, wherein the ring atoms are carbon, and one to 5 ring atoms is a heteroatom such as nitrogen, sulfur or oxygen. In some embodiments, to the extent not specified otherwise, heterocyclyl includes 3- to 7-membered monocycles having one or more heteroatoms selected from nitrogen, sulfur or oxygen. In some embodiments, heterocyclyl includes 4- to 6-membered monocycles having one or more heteroatoms selected from nitrogen, sulfur or oxygen. In some embodiments, heterocyclyl includes 3-membered monocycles. In some embodiments, heterocyclyl includes 4-membered monocycles. In some embodiments, heterocyclyl includes 5-6 membered monocycles. In some embodiments, the heterocyclyl group includes 0 to 3 double bonds. In any of the foregoing embodiments, heterocyclyl includes 1, 2, 3 or 4 heteroatoms. Any nitrogen or sulfur heteroatom may optionally be oxidized (e.g., NO, SO, SO₂), and any nitrogen heteroatom may optionally be quaternized (e.g., [NR₄]⁺Cl⁻, [NR₄]⁺OH⁻). Representative examples of heterocyclyls include oxiranyl, aziridinyl, thiiranyl, azetidinyl, oxetanyl, thietanyl, 1,2-dithietanyl, 1,3-dithietanyl, pyrrolidinyl, dihydro-1H-pyrrolyl, dihydrofuranyl, tetrahydropyranyl, dihydrothienyl, tetrahydrothienyl, imidazolidinyl, piperidinyl, piperazinyl, morpholinyl, thiomorpholinyl, 1,1-dioxo-thiomorpholinyl, dihydropyranyl, tetrahydropyranyl, hexahydrothiopyranyl, hexahydropyrimidinyl, oxazinanyl, thiazinanyl, thioxanyl, homopiperazinyl, homopiperidinyl, azepanyl, oxepanyl, thiepanyl, oxazepinyl, oxazepanyl, diazepanyl, 1,4-diazepanyl, diazepinyl, thiazepinyl, thiazepanyl, tetrahydrothiopyranyl, oxazolidinyl, thiazolidinyl, isothiazolidinyl, 1,1-dioxoisothiazolidinonyl, oxazolidinonyl, imidazolidinonyl, 4,5,6,7-tetrahydro[2H]indazolyl, tetrahydrobenzoimidazolyl, 4,5,6,7-tetrahydrobenzo[d]imidazolyl, 1,6-dihydroimidazol[4,5-d]pyrrolo[2,3-b]pyridinyl, thiazinyl, thiophenyl, oxazinyl, thiadiazinyl, oxadiazinyl, dithiazinyl, dioxazinyl, oxathiazinyl, thiatriazinyl, oxatriazinyl, dithiadiazinyl, imidazolinyl, dihydropyrimidyl, tetrahydropyrimidyl, 1-pyrrolinyl, 2-pyrrolinyl, 3-pyrrolinyl, indolinyl, thiapyranyl, 2H-pyranyl, 4H-pyranyl, dioxanyl, 1,3-dioxolanyl, pyrazolinyl, pyrazolidinyl, dithianyl, dithiolanyl, pyrimidinonyl, pyrimidindionyl, pyrimidin-2,4-dionyl, piperazinonyl, piperazindionyl, pyrazolidinylimidazolinyl, 3-azabicyclo[3.1.0]hexanyl, 3,6-diazabicyclo[3.1.1]heptanyl, 6-azabicyclo[3.1.1]heptanyl, 3-azabicyclo[3.1.1]heptanyl, 3-azabicyclo[4.1.0]heptanyl, azabicyclo[2.2.2]hexanyl, 2-azabicyclo[3.2.1]octanyl, 8-azabicyclo[3.2.1]octanyl, 2-azabicyclo[2.2.2]octanyl, 8-azabicyclo[2.2.2]octanyl, 7-oxabicyclo[2.2.1]heptane, azaspiro[3.5]nonanyl, azaspiro[2.5]octanyl, azaspiro[4.5]decanyl, 1-azaspiro[4.5]decan-2-only, azaspiro[5.5]undecanyl, tetrahydroindolyl, octahydroindolyl, tetrahydroisoindolyl, tetrahydroindazolyl, 1,1-dioxohexahydrothiopyranyl. Examples of 5-membered heterocyclyls containing a sulfur or oxygen atom and one to three nitrogen atoms are thiazolyl, including thiazol-2-yl and thiazol-2-yl N-oxide, thiadiazolyl, including 1,3,4-thiadiazol-5-yl and 1,2,4-thiadiazol-5-yl, oxazolyl, for example oxazol-2-yl, and oxadiazolyl, such as 1,3,4-oxadiazol-5-yl, and 1,2,4-oxadiazol-5-yl. Example 5-membered ring heterocyclyls containing 2 to 4 nitrogen atoms include imidazolyl, such as imidazol-2-yl; triazolyl, such as 1,3,4-triazol-5-yl; 1,2,3-triazol-5-yl, 1,2,4-triazol-5-yl, and tetrazolyl, such as 1H-tetrazol-5-yl. Representative examples of benzo-fused 5-membered heterocyclyls are benzoxazol-2-yl, benzthiazol-2-yl and benzimidazol-2-yl. Example 6-membered heterocyclyls contain one to three nitrogen atoms and optionally a sulfur or oxygen atom, for example pyridyl, such as pyrid-2-yl, pyrid-3-yl, and pyrid-4-yl; pyrimidyl, such as pyrimid-2-yl and pyrimid-4-yl; triazinyl, such as 1,3,4-triazin-2-yl and 1,3,5-triazin-4-yl; pyridazinyl, in particular pyridazin-3-yl, and pyrazinyl.

Thus, the term heterocyclic embraces N-heterocyclyl groups which as used herein refer to a heterocyclyl group containing at least one nitrogen and where the point of attachment of the heterocyclyl group to the rest of the molecule is through a nitrogen atom in the heterocyclyl group. To the extent not specified otherwise with respect to any one or more groups in the compounds of formula (I), representative examples of N-heterocyclyl groups include 1-morpholinyl, 1-piperidinyl, 1-piperazinyl, 1-pyrrolidinyl, pyrazolidinyl, imidazolinyl and imidazolidinyl. The term heterocyclic also embraces C-heterocyclyl groups which as used herein refer to a heterocyclyl group containing at least one heteroatom and where the point of attachment of the heterocyclyl group to the rest of the molecule is through a carbon atom in the heterocyclyl group. To the extent not specified otherwise, representative examples of C-heterocyclyl radicals include 2-morpholinyl, 2- or 3- or 4-piperidinyl, 2-piperazinyl, and 2- or 3-pyrrolidinyl. The term heterocyclic also embraces heterocyclylalkyl groups which as disclosed above refer to a group of the formula —R^{c}-heterocyclyl where R^{c} is an alkylene chain. The term heterocyclic also embraces heterocyclylalkoxy groups which as used herein refer to a radical bonded through an oxygen atom of the formula —O—R^{c}-heterocyclyl where R^{c} is an alkylene chain.

As used herein, the term “aryl” used alone or as part of a larger moiety (e.g., “aralkyl”, wherein the terminal carbon atom on the alkyl group is the point of attachment, e.g., a benzyl group), “aralkoxy” wherein the oxygen atom is the point of attachment, or “aroxyalkyl” wherein the point of attachment is on the aryl group) refers to a group that includes monocyclic, bicyclic or tricyclic, carbon ring system, that includes fused rings, wherein at least one ring in the system is aromatic. In some embodiments, the aralkoxy group is a benzoxy group. The term “aryl” may be used interchangeably with the term “aryl ring”. In one embodiment, to the extent not specified otherwise, aryl includes groups having 6-18 carbon atoms. In another embodiment, aryl includes groups having 6-10 carbon atoms. Examples of aryl groups include phenyl, naphthyl, anthracyl, biphenyl, phenanthrenyl, naphthacenyl, 1,2,3,4-tetrahydronaphthalenyl, 1H-indenyl, 2,3-dihydro-TH-indenyl, naphthyridinyl, and the like, which may be substituted or independently substituted by one or more substituents described herein. A particular aryl is phenyl. In some embodiments, to the extent not specified otherwise, an aryl group includes an aryl ring fused to one or more (e.g., 1, 2 or 3) different cyclic groups (e.g., carbocyclic rings or heterocyclic rings), where the radical or point of attachment is on the aryl ring.

Thus, the term aryl embraces aralkyl groups (e.g., benzyl) which as disclosed above refer to a group of the formula —R_{c}-aryl where R^{c} is an alkylene chain such as methylene or ethylene. In some embodiments, to the extent not specified otherwise with respect to any one or more groups in the compounds of formula (I), the aralkyl group is an optionally substituted benzyl group. The term aryl also embraces aralkoxy groups which as used herein refer to a group bonded through an oxygen atom of the formula —O—R^{c}-aryl where R^{c} is an alkylene chain such as methylene or ethylene.

As used herein, the term “heteroaryl” used alone or as part of a larger moiety (e.g., “heteroarylalkyl” (also “heteroaralkyl”), or “heteroarylalkoxy” (also “heteroaralkoxy”), refers to a monocyclic, bicyclic or tricyclic ring system having 5 to 14 ring atoms, wherein at least one ring is aromatic and contains at least one heteroatom. In one embodiment, to the extent not specified otherwise with respect to any one or more groups in the compounds of formula (I), heteroaryl includes 5-6 membered monocyclic aromatic groups where one or more ring atoms is nitrogen, sulfur or oxygen that is independently optionally substituted. Representative examples of heteroaryl groups include thienyl, furyl, imidazolyl, pyrazolyl, thiazolyl, isothiazolyl, oxazolyl, isoxazolyl, triazolyl, thiadiazolyl, oxadiazolyl, tetrazolyl, thiatriazolyl, oxatriazolyl, pyridyl, pyrimidyl, imidazopyridyl, pyrazinyl, pyridazinyl, triazinyl, tetrazinyl, tetrazolo[1,5-b]pyridazinyl, purinyl, deazapurinyl, benzoxazolyl, benzofuryl, benzothiazolyl, benzothiadiazolyl, benzotriazolyl, benzoimidazolyl, indolyl, 1,3-thiazol-2-yl, 1,3,4-triazol-5-yl, 1,3-oxazol-2-yl, 1,3,4-oxadiazol-5-yl, 1,2,4-oxadiazol-5-yl, 1,3,4-thiadiazol-5-yl, 1H-tetrazol-5-yl, 1,2,3-triazol-5-yl, and pyrid-2-yl N-oxide. The term “heteroaryl” also includes groups in which a heteroaryl is fused to one or more (e.g., 1, 2 or 3) cyclic (e.g., carbocyclyl, or heterocyclyl) rings, where the radical or point of attachment is on the heteroaryl ring. Nonlimiting examples include indolyl, indolizinyl, isoindolyl, benzothienyl, benzothiophenyl, methylenedioxyphenyl, benzofuranyl, dibenzofuranyl, indazolyl, benzimidazolyl, benzodioxazolyl, benzthiazolyl, quinolyl, isoquinolyl, cinnolinyl, phthalazinyl, quinazolinyl, quinoxalinyl. 4H-quinolizinyl, carbazolyl, acridinyl, phenazinyl, phenothiazinyl, phenoxazinyl, tetrahydroquinolinyl, tetrahydroisoquinolinyl and pyrido[2,3-b]-1,4-oxazin-3(4H)-one. A heteroaryl group may be mono-, bi- or tri-cyclic. In some embodiments, a heteroaryl group includes a heteroaryl ring fused to one or more (e.g., 1, 2 or 3) different cyclic groups (e.g., carbocyclic rings or heterocyclic rings), where the radical or point of attachment is on the heteroaryl ring, and in some embodiments wherein the point of attachment is a heteroatom contained in the heterocyclic ring.

The term heteroaryl also embraces N-heteroaryl groups which as used herein refers to a heteroaryl group, as defined above, and which contains at least one nitrogen atom and where the point of attachment of the N-heteroaryl group to the rest of the molecule is through a nitrogen atom in the heteroaryl group. The term heteroaryl further embraces C-heteroaryl groups which as used herein refer to a heteroaryl group as defined above and where the point of attachment of the heteroaryl group to the rest of the molecule is through a carbon atom in the heteroaryl group. The term heteroaryl further embraces heteroarylalkyl groups which as disclosed above refer to a group of the formula —R^{c}-heteroaryl, wherein R^{c} is an alkylene chain as defined above. The term heteroaryl further embraces heteroaralkoxy (or heteroarylalkoxy) groups which as used herein refer to a group bonded through an oxygen atom of the formula —O—R^{c}-heteroaryl, where R^{c} is an alkylene group as defined above.

To the extent not disclosed otherwise with respect to any one or more groups in the compounds of formula (I), representative examples of substituents may thus include alkyl, substituted alkyl (e.g., C₁-C₆, C₁-C₅, C₁-C₄, C₁-C₃, C₁-C₂, C₁), alkoxy (e.g., C₁-C₆, C₁-C₅, C₁-C₄, C₁-C₃, C₁-C₂, C₁), substituted alkoxy (e.g., C₁-C₆, C₁-C₅, C₁-C₄, C₁-C₃, C₁-C₂, C₁), haloalkyl (e.g., CF₃), alkenyl (e.g., C₂-C₆, C₂-C₅, C₂-C₄, C₂-C₃, C₂), substituted alkenyl (e.g., C₂-C₆, C₂-C₅, C₂-C₄, C₂-C₃, C₂), alkynyl (e.g., C₂-C₆, C₂-C₅, C₂-C₄, C₂-C₃, C₂), substituted alkynyl (e.g., C₂-C₆, C₂-C₅, C₂-C₄, C₂-C₃, C₂), cyclic (e.g., C₃-C₁₂, C₅-C₆), substituted cyclic (e.g., C₃-C₁₂, C₅-C₆), carbocyclic (e.g., C₃-C₁₂, C₅-C₆), substituted carbocyclic (e.g., C₃-C₁₂, C₅-C₆), heterocyclic (e.g., C₃-C₁₂, C₅-C₆), substituted heterocyclic (e.g., C₃-C₁₂, C₅-C₆), aryl (e.g., benzyl and phenyl), substituted aryl (e.g., substituted benzyl or phenyl), heteroaryl (e.g., pyridyl or pyrimidyl), substituted heteroaryl (e.g., substituted pyridyl or pyrimidyl), aralkyl (e.g., benzyl), substituted aralkyl (e.g., substituted benzyl), halo, hydroxyl, aryloxy (e.g., C₆-C₁₂, C₆), substituted aryloxy (e.g., C₆-C₁₂, C₆), alkylthio (e.g., C₁-C₆), substituted alkylthio (e.g., C₁-C₆), arylthio (e.g., C₆-C₁₂, C₆), substituted arylthio (e.g., C₆-C₁₂, C₆), cyano, carbonyl, substituted carbonyl, carboxyl, substituted carboxyl, amino, substituted amino, amido, substituted amido, thio, substituted thio, sulfinyl, substituted sulfinyl, sulfonyl, substituted sulfonyl, sulfinamide, substituted sulfinamide, sulfonamide, substituted sulfonamide, urea, substituted urea, carbamate, substituted carbamate, amino acid, and peptide groups.

In one aspect, compounds of the disclosure are represented by formula (I):

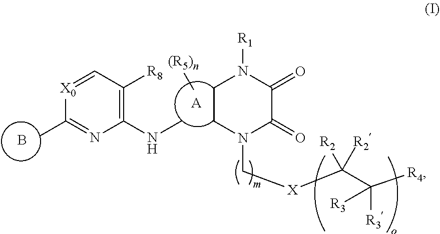

or a pharmaceutically acceptable salt or stereoisomer thereof, wherein:

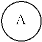

is phenyl or pyridyl;

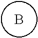

is

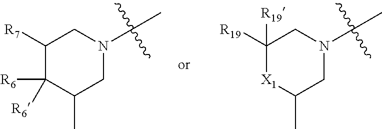

X is a bond, (C₃-C₆) carbocyclyl, (C₃-C₆) carbocyclyl(C═O), or SO₂;
X₀ is N or CCN,
X₁ is CH₂, S, CHF, CHCl, CHOH, or CF₂;
R₁ is (C₁-C₆) alkyl, (C₁-C₆) hydroxyalkyl, (C₁-C₆) aminoalkyl, (C₃-C₆) carbocyclyl, 4- to 6-membered heterocyclyl, (C₁-C₆) alkyl-(C₃-C₆) carbocyclyl, or (C₁-C₆) alkyl-4- to 6-membered heterocyclyl, wherein said alkyl, hydroxyalkyl, aminoalkyl, carbocyclyl, or heterocyclyl is further optionally substituted by one or more, identical or different R₁ₐ groups, wherein R₁ₐ is deuterium, (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl;
R₂ and R_{2′} are each independently H, (C₁-C₃) alkyl, (C₁-C₃) hydroxyalkyl, or (C₁-C₃) aminoalkyl, or R₂ and R_{2′}, together with the same carbon atom to which they are attached, form (C₃-C₆) carbocyclyl or 4- to 6-membered heterocyclyl, wherein said alkyl, hydroxyalkyl, aminoalkyl, carbocyclyl, or heterocyclyl is optionally substituted by one or more, identical or different R₂ₐ groups, wherein R₂ₐ is (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl;
R₃ and R_{3′} are each independently H, (C₁-C₃) alkyl, (C₁-C₃) hydroxyalkyl, or (C₁-C₃) aminoalkyl, or
R₃ and R_{3′}, together with the same carbon atom to which they are attached, form C═O, (C₃-C₆) carbocyclyl, 4- to 6-membered heterocyclyl, wherein said carbocyclyl or heterocyclyl is optionally substituted by one or more, identical or different R₃ₐ groups, wherein each R₃ₐ is independently (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl, or wherein two R₃ₐ groups, together with the same carbon atom to which they are attached, form C═O,
or wherein R₂ or R_{2′} and R₃ or R_{3′}, together with the same carbon atom to which they are attached, form (C₃-C₆) carbocyclyl or 4- to 6-membered heterocyclyl, wherein said carbocyclyl or heterocyclyl is optionally substituted by one or more, identical or different R₂ₐ groups;
R₄ is H, OH, NH—(C₁-C₆) alkyl, NH—(C₁-C₆) hydroxyalkyl, NH—(C₁-C₆) aminoalkyl, NH—(C₃-C₆) carbocyclyl, NH-4- to 6-membered heterocyclyl, NH—(C₁-C₆) alkyl-(C₃-C₆) carbocyclyl, NH—(C₁-C₆) alkyl-4- to 6-membered heterocyclyl, wherein said alkyl, hydroxyalkyl, aminoalkyl, carbocyclyl, or heterocyclyl is further optionally substituted by one or more, identical or different R₄ₐ groups, wherein each R₄ₐ is independently (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl;
R₅ is halogen or (C₁-C₃) alkoxy;
R₆ and R_{6′} are each independently H or halogen;
R₇ is H, halogen, (C₁-C₄) alkyl, (C₁-C₄) alkoxy, (C₁-C₄) haloalkyl, (C₁-C₄) haloalkoxy, CN, NO₂, OH, (C₂-C₄) alkenyl, (C₂-C₄) alkynyl, or —R₇ₐR_{7b}, wherein R₇ₐ is absent or O, N(R₇ₐ₁)(CR₇ₐ₁R₇ₐ₂)_{q}, S, SO, SO₂, C(O), C(O)O. OC(O), C(O)N(R₇ₐ₁), N(R₇ₐ₁)C(O), N(R₇ₐ₁)C(O)N(R₇ₐ₁), N(R₇ₐ₁)C(O)O, OC(O)N(R₇ₐ₁), S(O)₂N(R₇ₐ₁), N(R₇ₐ₁)SO₂, wherein R₇ₐ₁ and R₇ₐ₂ are each independently H or (C₁-C₄) alkyl, and q is 0, 1, 2, and Ry, is H, (C₁-C₆) alkyl, (C₆-C₁₀) aryl, (C₃-C₆) cycloalkyl, (C₂-C₄) alkenyl, (C₁-C₄) alkynyl, (C₃-C₆) cycloalkenyl, wherein said alkyl, aryl, cycloalkyl, alkenyl, alkynyl, or cycloalkenyl is optionally further substituted by one or more substituent groups independently selected from oxo, (C₁-C₄) alkyl, (C₁-C₄) cycloalkyl, halogen, (C₁-C₄) haloalkyl, (C₁-C₄) haloalkoxy, (C₁-C₄) hydoxyalkyl, amino, CN, OH, amido, carboxy, carbamoyl, sulphamoyl, mercapto, C(O)NR_{7b1}R_{7b2}, R_{7b1}R_{7b2}, or OR_{7b1}, wherein R_{7b1} and R_{7b2} each is independently selected from hydrogen, (C₁-C₄) alkyl, (C₃-C₆) cycloalkyl, wherein said oxo, alkyl, cycloalkyl, haloalkyl, haloalkoxy, hydoxyalkyl, amino, CN OH, amido carboxy, carbamoyl, sulphamoyl, or mercapto is optionally further substituted by R_{7b3}—R_{7b4}, wherein R_{7b3} is absent or (C₁-C₅) alkylene optionally substituted by one or more substituents selected from (C₁-C₂) alkyl or oxo, and R_{7b4} is (C₆-C₁₀) aryl, 5- to 12-membered heteroaryl, 4- to 12-membered heterocyclyl, (C₃-C₆) carbocyclyl, halogen, (C₁-C₄) haloalkyl, (C₁-C₄) haloalkoxy, CN, OH, (C₁-C₄) alkoxy, C(O)R_{7b4a}, COOR_{7b4b}, C(O)NR_{7b4a}R_{7b4b}, or NR_{7b4a}R_{7b4b}, wherein: R_{7b4a} and R_{7b4b} are each independently selected from H or (C₁-C₄) alkyl; said aryl, heteroaryl, heterocyclyl, or carbocyclyl is optionally further substituted by one or more substituent groups independently selected from (C₁-C₄)alkyl, halogen, (C₁-C₄) haloalkyl, amino, CN or OH; and said heterocyclyl or heteroaryl contains at least one nitrogen atom and is linked via nitrogen, wherein said heterocyclyl group is optionally and independently substituted by one or more, identical or different groups independently selected from R₉, and said heteroaryl group is optionally and independently substituted by one or more, identical or different group independently selected from R₁₀;
R₈ is halogen or CN;
R₉ is ═O, CN, C≡CH, OH, COOH, halogen, (C₁-C₆) alkyl, (C₁-C₆) alkoxy, (C₁-C₆) haloalkyl, 5 or 6 membered heteroaryl, phenyl, N(R₁₁R₁₂), C(O)—R₁₃, C(O)N(R₁₄R₁₅), or 5- to 8-membered heterocyclyl, wherein said (C₁-C₆) alkyl is optionally substituted with COOH, OH, COO—(C₁-C₆) alkyl, —CON((C₁-C₆) alkyl)₂, (C₁-C₆) alkoxy, N((C₁-C₃) alkyl)₂, phenyl, or 5- or 6-membered heterocyclyl, and said heteroaryl, phenyl, or heterocyclyl is optionally substituted with one group selected from (C₁-C₆) alkyl;
R₁₀ is COOH, (C₁-C₆) alkyl, C(O)—R₁₆, or C(O)N(R₁₇)(R₁₈);
Rn is H or (C₁-C₄) alkyl;
R₁₂ is H, (C₁-C₄) alkyl, (C₁-C₄) haloalkyl, 6-membered heterocyclyl, or 6-membered heteroaryl;
R₁₃ is (C₁-C₃) alkyl-N((C₁-C₃) alkyl)₂ or 5- or 6-membered heterocyclyl, wherein said heterocyclyl is optionally substituted with (C₁-C₃) alkyl;
R₁₄ is hydrogen or (C₁-C₃) alkyl;
R₁₅ is (C₁-C₆) alkyl optionally substituted with NH₂, (C₁-C₆) alkoxy, O—(C₁-C₆) alkyl-NH₂, or O—(C₁-C₆) alkyl-O—(C₁-C₆) alkyl-NH₂, or R₁₅ is a 6-membered heterocyclyl optionally substituted with (C₁-C₃) alkyl;
R₁₆ is a 6-membered heterocyclyl optionally substituted with (C₁-C₃) alkyl;
Rn and Rig are each independently H or (C₁-C₃) alkyl;
R₁₉ is H, ═O, —CN, —C≡CH, —OH, —SH, —NH₂, —COOH, halo, (C₁-C₆)alkyl, —O—(C₁-C₆)alkyl, (C₁-C₆)haloalkyl, amido, carboxy, carbamoyl, sulfamoyl, phenyl, 5- to 8-membered heterocyclyl, —NR₂₀R₂₁, —C(O)R₂₂, —C(O)NR₂₃R₂₄, or L₁Y₁, wherein said alkyl, phenyl, or heterocyclyl is optionally substituted with one or more groups selected from halo, —COOH, —OH, —NH₂, (C₁-C₆)alkyl, —C(O)O—(C₁-C₆)alkyl, —C(O)N(C₁-C₆ alkyl)₂, —O—(C₁-C₆)alkyl, —N(C₁-C₃ alkyl)₂, phenyl, and 4- to 6-membered heterocyclyl, optionally substituted with one or more groups selected from halo and (C₁-C₆)alkyl,
L₁ is absent, (C₁-C₆)alkylene or (C₃-C₇)carbocyclyl; wherein said alkylene or carbocyclyl is further optionally substituted by one or more, identical or different R₂₅ groups, or L₁ is (C₂-C₄)alkylene which is bound to R₂₆ to form a 4- to 6-membered heterocyclyl group; and
Y₁ is —CN, —OH, halo, (C₁-C₄)alkoxy, (C₁-C₄)haloalkyl, (C₁-C₄)haloalkoxy, 4- to 7-membered heterocyclyl, (C₃-C₆)carbocyclyl, —NR₂₆R₂₇, —C(O)R₂₂, —C(O)NR₂₃R₂₄, wherein said alkyl, carbocyclyl, or heterocyclyl is further optionally substituted by one or more, identical or different groups selected from (C₁-C₄)alkyl, halo, (C₁-C₄)haloalkyl, —CN, —OH, and —NH₂;
R₁₉′ is absent, H, —CN, —C≡CH, —OH, —SH, —NH₂, —COOH, halo, (C₁-C₆)alkyl, —O—(C₁-C₆)alkyl, (C₁-C₆)haloalkyl, amido, carboxy, carbamoyl, sulfamoyl, phenyl, 5- to 8-membered heterocyclyl, —NR₂₀R₂₁, —C(O)R₂₂, or —C(O)NR₂₃R₂₄; wherein said alkyl, phenyl, or heterocyclyl is further optionally substituted by one or more, identical or different R₂₅ groups, or
R₁₉′ and L₁ together with the same carbon atom to which they are attached form a spiro (C₃-C₇)carbocyclyl group or a 4- to 7-membered heterocyclyl group; wherein said carbocyclyl or heterocyclyl is further optionally substituted by one or more, identical or different R₂₅ groups;
R₂₀ is hydrogen, (C₁-C₄)alkyl, or (C₃-C₆)cycloalkyl;
R₂₁ is hydrogen, (C₁-C₄)alkyl, (C₁-C₄)haloalkyl, (C₃-C₆)cycloalkyl, or 6-membered heterocyclyl;
R₂₂ is —(C₁-C₃)alkyl-N(C₁-C₃ alkyl)₂, (C₃-C₆)cycloalkyl, or 5- to 6-membered heterocyclyl, wherein said heterocyclyl is optionally substituted with (C₁-C₃)alkyl;
R₂₃ is hydrogen, (C₁-C₃)alkyl, or (C₃-C₆)cycloalkyl;
R₂₄ is (C₃-C₆)cycloalkyl or (C₁-C₆)alkyl optionally substituted with —NH₂, —O—(C₁-C₆)alkyl, —O—(C₁-C₆)alkyl-NH₂, or —O—(C₁-C₆)alkyl-O—(C₁-C₆)alkyl-NH₂;
each R₂₅ is independently oxo, alkyl, alkenyl, alkynyl, halo, haloalkyl, carbocyclyl, heterocyclyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylanminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, or phosphinyl;
R₂₆ and R₂₇ are each independently hydrogen, (C₁-C₆)alkyl, (C₃-C₇)carbocyclyl, 4- to 7-membered heterocyclyl, (C₆-C₁₀)aryl, or monocyclic or bicyclic 5- to 10-membered heteroaryl;
wherein said alkyl, carbocyclyl, heterocyclyl, aryl or heteroaryl is further optionally substituted by one or more, identical or different R₂₅ groups, or R₂₆ and R₂₇ together with the nitrogen atom to which they are attached form a 3- to 7-membered heterocyclyl, wherein said heterocyclyl is further optionally substituted by one or more, identical or different R₂₅ groups;
m is 0, 1, or 2;
n is 0, 1, 2, or 3; and
o is 0 or 1.

In some embodiments, wherein

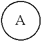

is phenyl and

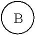

is

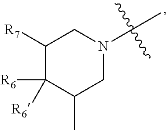

compounds of the disclosure are represented by structure I-1a:

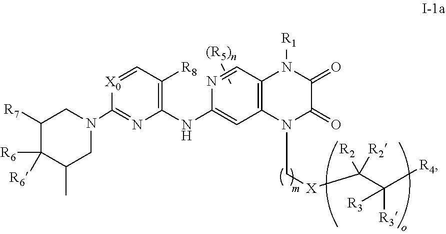

or a pharmaceutically acceptable salt or stereoisomer thereof.

In some embodiments, wherein

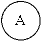

is pyridyl and

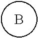

is

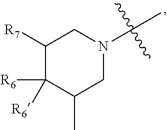

compounds of the disclosure are represented by any one of structures I-2a to I-4a:

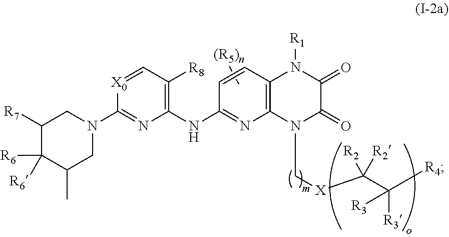

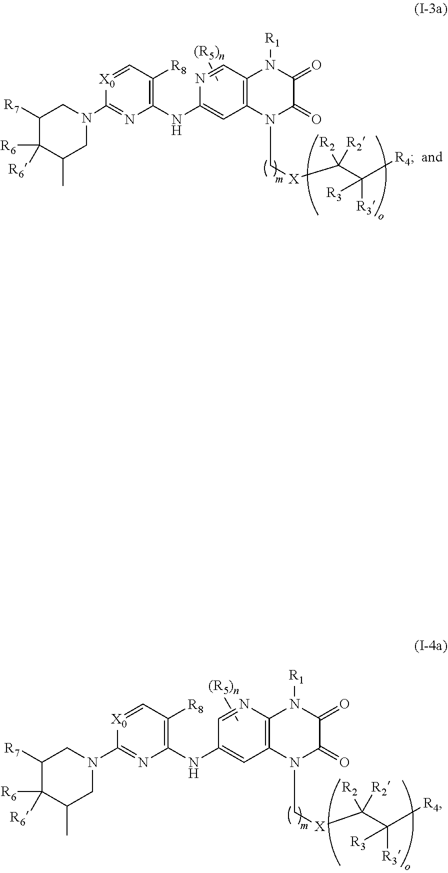

or a pharmaceutically acceptable salt or stereoisomer thereof.

In some embodiments, R₇ is optionally substituted (C₁-C₄) alkyl. In some embodiments, R₇ is methyl or

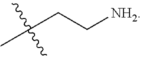 [SMILES: *CCN]

In some embodiments, R₇ is amino or OH.

In some embodiments, wherein

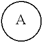

is phenyl and

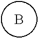

is

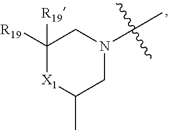

compounds of the disclosure are represented by structure I-1b:

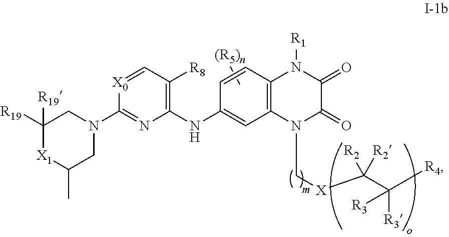

or a pharmaceutically acceptable salt or stereoisomer thereof.

In some embodiments, wherein

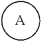

is pyridyl and

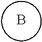

is

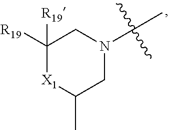

compounds of the disclosure are represented by any one of structures I-2b to I-4b:

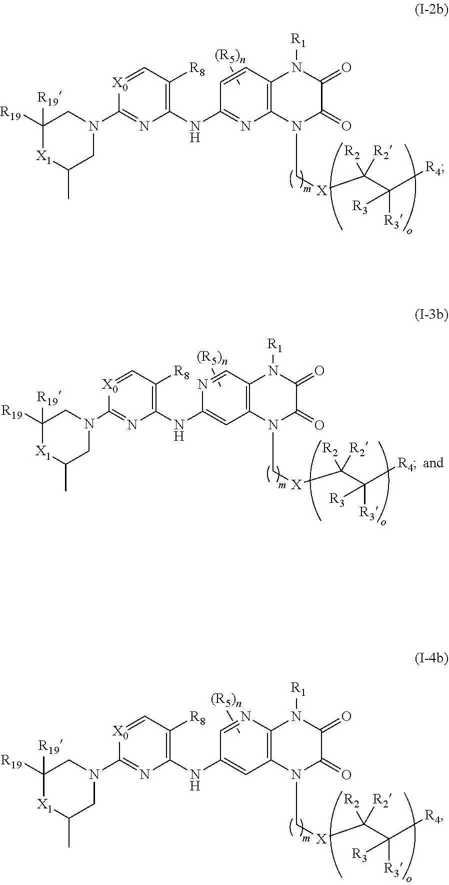

or a pharmaceutically acceptable salt or stereoisomer thereof.

In some embodiments, X₁ is CH₂, CHF, CHOH, or CF₂.

In some embodiments, R₁₉ and R₁₉′ are each independently H, OH, methyl, amino,

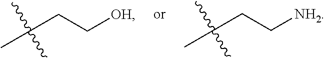

In some embodiments, R₈ is halogen. In some embodiments, R₈ is Cl or F.

In some embodiments, R₁ is optionally substituted (C₁-C₆) alkyl. In some embodiments, R₁ is methyl.

In some embodiments, R₁ is optionally substituted (C₁-C₆) hydroxyalkyl. In some embodiments, R₁ is

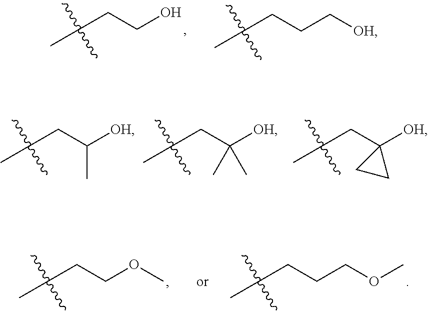

In some embodiments, R₁ is optionally substituted (C₁-C₆) aminoalkyl. In some embodiments, R₁ is

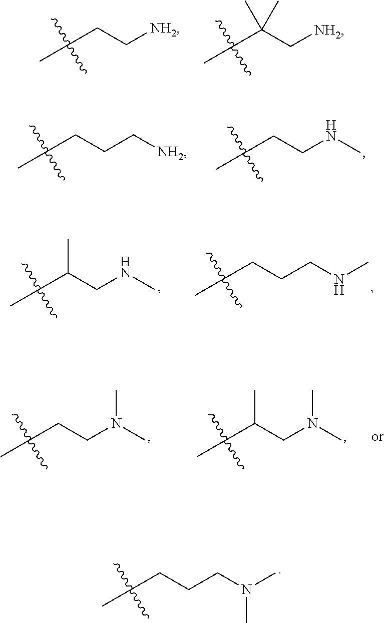

In some embodiments, R₁ is optionally substituted 4- to 6-membered heterocyclyl. In some embodiments, R₁ is

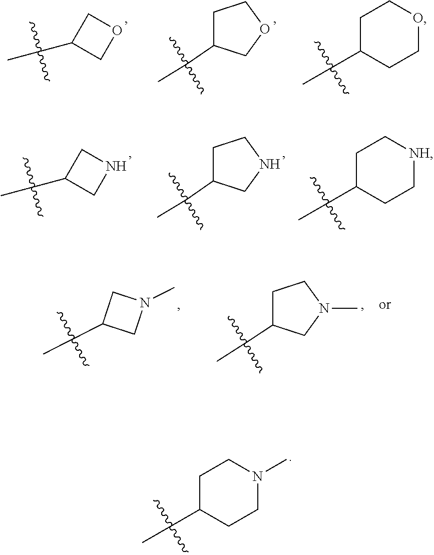

In some embodiments, R₁ is optionally substituted (C₁-C₆) alkyl-4- to 6-membered heterocyclyl. In some embodiments, R₁ is

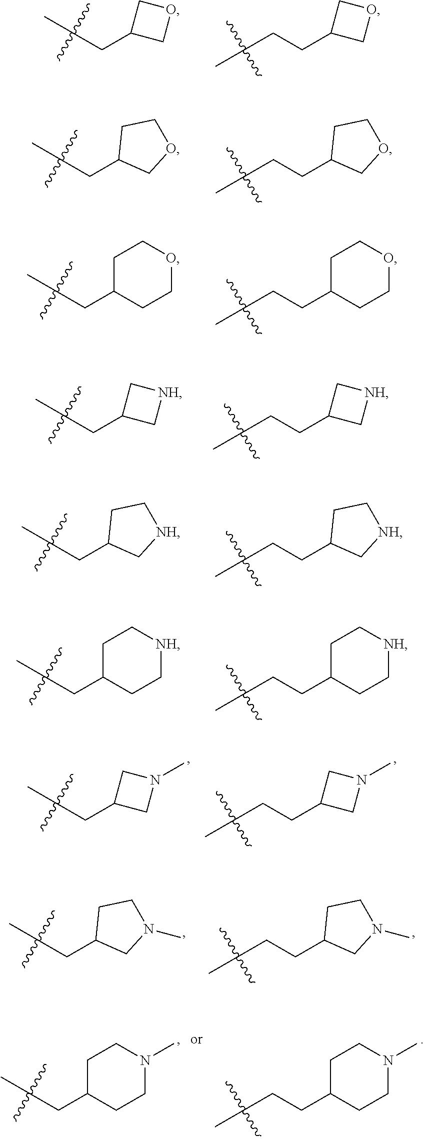

In some embodiments, X is a bond or (C₃-C₆) carbocyclyl, m is 0 or 1, and o is 0 or 1.

In some embodiments, wherein

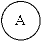

is phenyl,

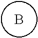

is

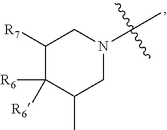

X is a bond, m is 0 or 1, and o is 1, compounds of the disclosure are represented by structure I-1a1 or I-1b1:

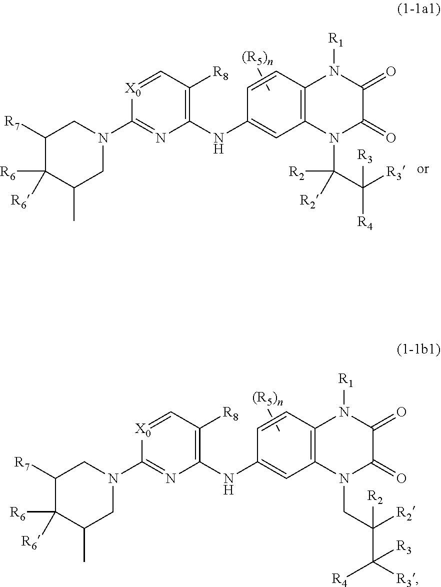

or a pharmaceutically acceptable salt or stereoisomer thereof.

In some embodiments, wherein

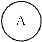

is phenyl,

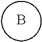

is

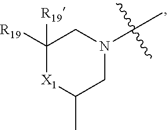

X is a bond, m is 0 or 1, and o is 1, compounds of the disclosure are represented by structure I-1a2 or I-1b2:

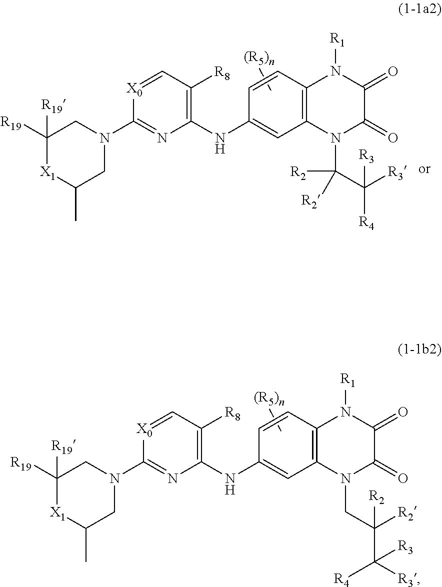

or a pharmaceutically acceptable salt or stereoisomer thereof.

In some embodiments, wherein

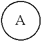

is pyridyl

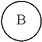

is

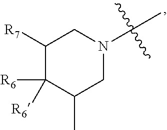

X is a bond, m is 0 or 1, and o is 1, compounds of the disclosure are represented by any one of structures I-2a1 to I-4a1 and I-2b1 to I-4b1:

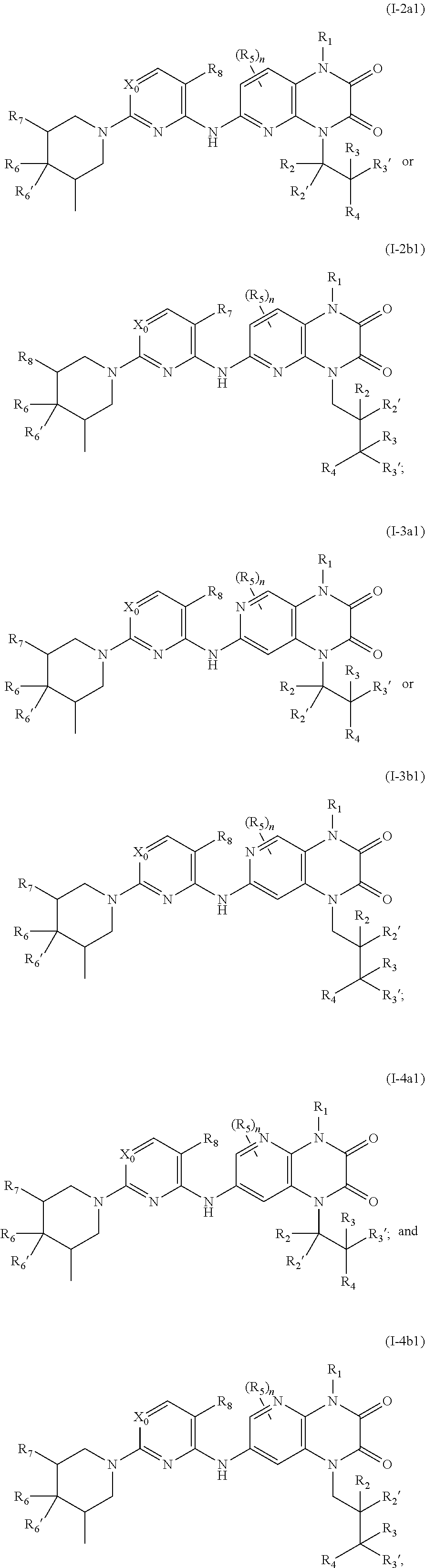

or a pharmaceutically acceptable salt or stereoisomer thereof.

In some embodiments, wherein

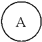

is pyridyl,

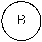

is

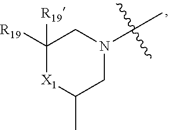

X is a bond, m is 0 or 1, and o is 1, compounds of the disclosure are represented by any one of structures I-2a2 to I-4a2 and I-2b2 to I-4b2:

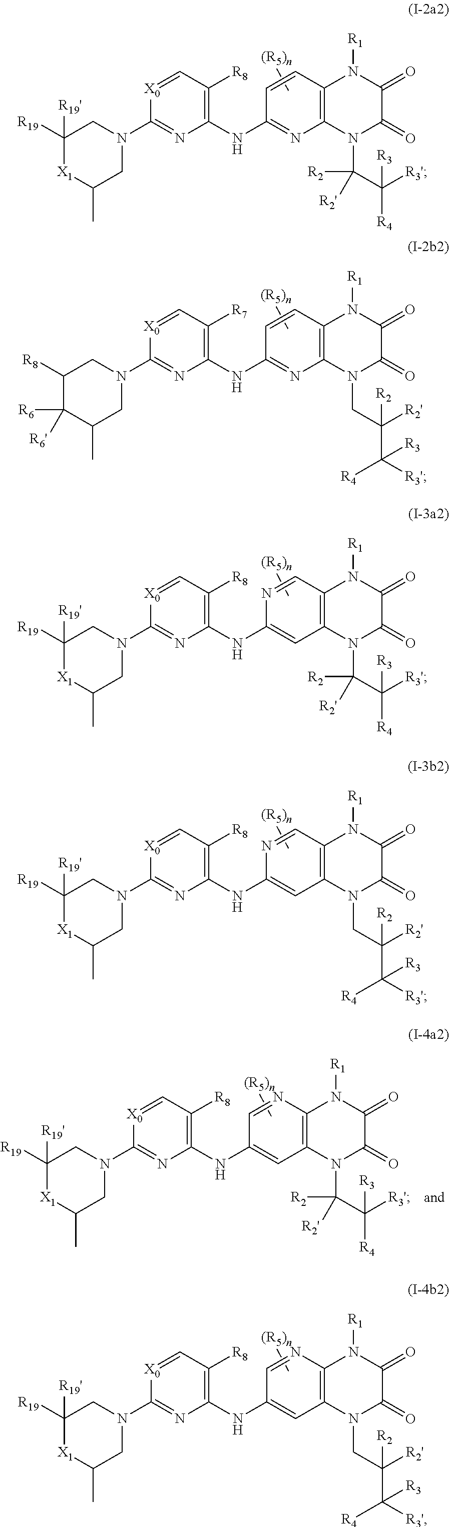

or a pharmaceutically acceptable salt or stereoisomer thereof.

In some embodiments, R₂ and R_{2′} are each independently H or methyl. In some embodiments both R₂ and R_{2′} are H. In some embodiments, R₂ and R_{2′}, together with the same carbon atom to which they are attached, form cyclopropyl.

In some embodiments, R₃ and R_{3′} are each independently H or methyl. In some embodiments, R₃ and R_{3′}, together with the same carbon atom to which they are attached, form C═O. In some embodiments, R₃ and R_{3′}, together with the same carbon atom to which they are attached, form optionally substituted 4- to 6-membered heterocyclyl. In some embodiments, two R₃ₐ groups, together with the same carbon atom to which they are attached, form C═O. In some embodiments, the optionally substituted 4-membered heterocyclyl is oxetane or N-methylazetidine. In some embodiment, the optionally substituted 5- or 6-membered heterocyclyl is carbamate. In some embodiments, R₂ or R_{2′} and R₃ or R_{3′}, together with the same carbon atom to which they are attached, form (C₃-C₆) carbocyclyl.

In some embodiments, R₄ is OH or H.

In some embodiments, R₂ and R₂, are each independently H or methyl; R₃ and R_{3′} are each independently H or methyl; and R₄ is OH or H. In some embodiments, R₂ and R_{2′} are each H; R₃ and R_{3′} are each independently H or methyl; and R₄ is OH.

In some embodiments, wherein

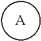

is phenyl,

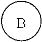

is

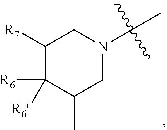

X is C₄ carbocyclyl(C═O), and m and o are 0, compounds of the present disclosure are represented by structure I-1c1:

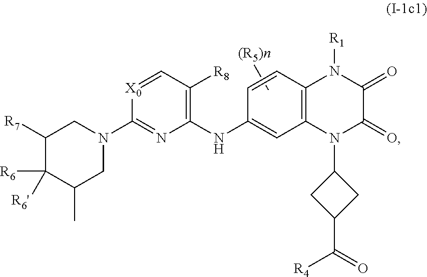

or a pharmaceutically acceptable salt or stereoisomer thereof.

In some embodiments, wherein

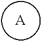

is phenyl,

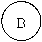

is

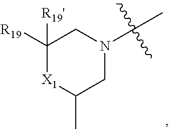

X is C₄ carbocyclyl(C═O), and m and o are 0, compounds of the present disclosure are represented by structure I-1c2:

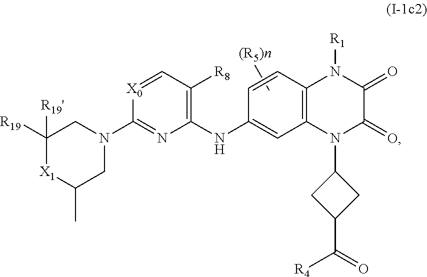

or a pharmaceutically acceptable salt or stereoisomer thereof.

In some embodiments, wherein

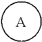

is pyridyl

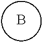

is

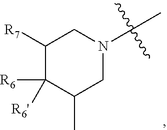

X is C₄ carbocyclyl(C═O), and m and o are 0, compounds of the present disclosure are represented by any one of structures I-2c1 to I-4c1:

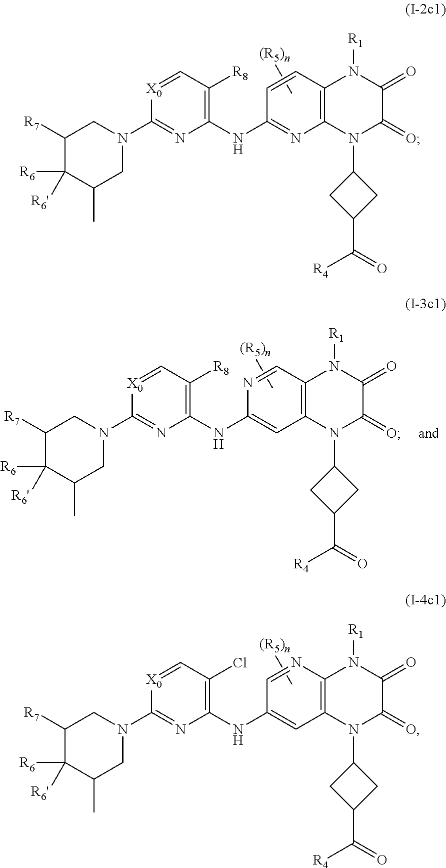

or a pharmaceutically acceptable salt or stereoisomer thereof.

In some embodiments, wherein

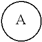

is pyridyl

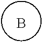

is

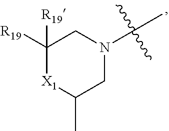

X is C₄ carbocyclyl(C═O), and m and o are 0, compounds of the present disclosure are represented by any one of structures I-2c2 to I-4c2:

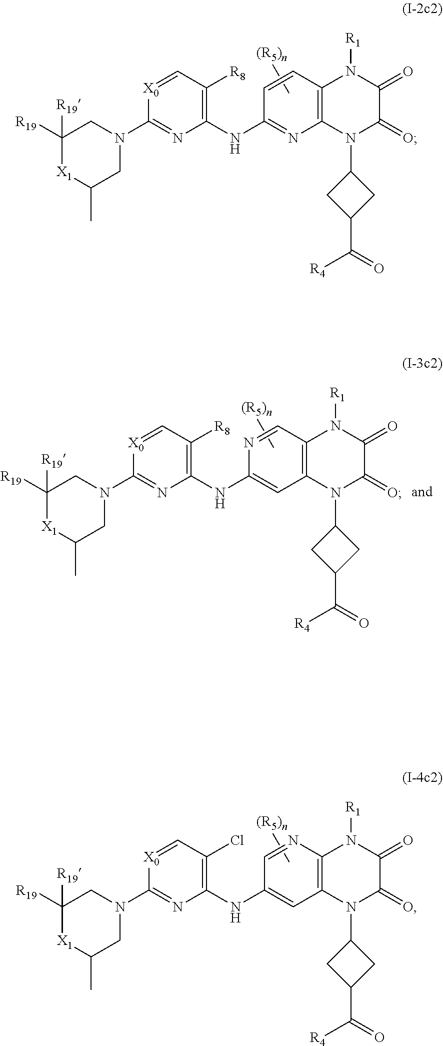

or a pharmaceutically acceptable salt or stereoisomer thereof.

In some embodiments, wherein

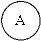

is phenyl,

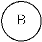

is

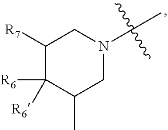

X is SO₂, m is 1 or 2, and o is 0, compounds of the present disclosure are represented by structure I-1d1:

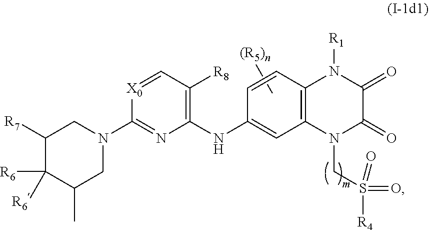

or a pharmaceutically acceptable salt or stereoisomer thereof.

In some embodiments, wherein

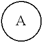

is phenyl,

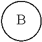

is

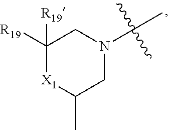

X is SO₂, m is 1 or 2, and o is 0, compounds of the present disclosure are represented by structure I-1d2:

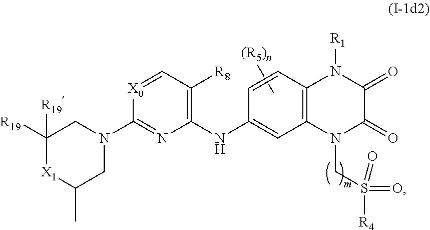

or a pharmaceutically acceptable salt or stereoisomer thereof.

In some embodiments, wherein

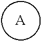

is pyridyl,

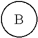

is

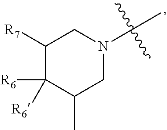

X is SO₂, m is 1 or 2, and o is 0, compounds of the present disclosure are represented by any one of structures I-2d1 to I-4d1:

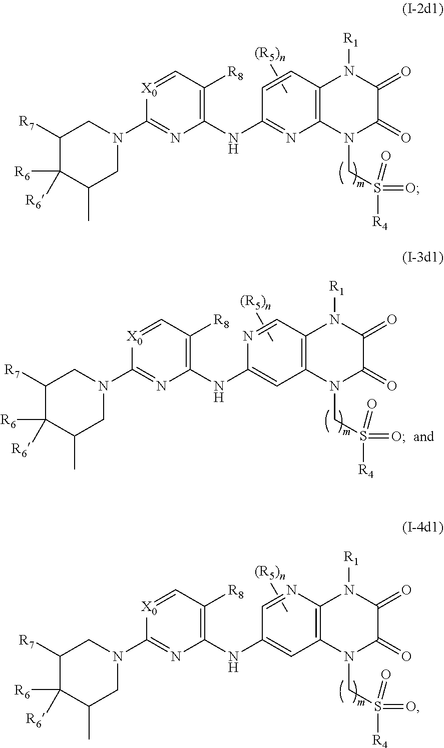

or a pharmaceutically acceptable salt or stereoisomer thereof.

In some embodiments, wherein

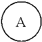

is pyridyl

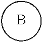

is

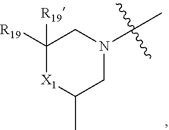

X is SO₂, m is 1 or 2, and o is 0, compounds of the present disclosure are represented by any one of structures I-2d2 to I-4d2:

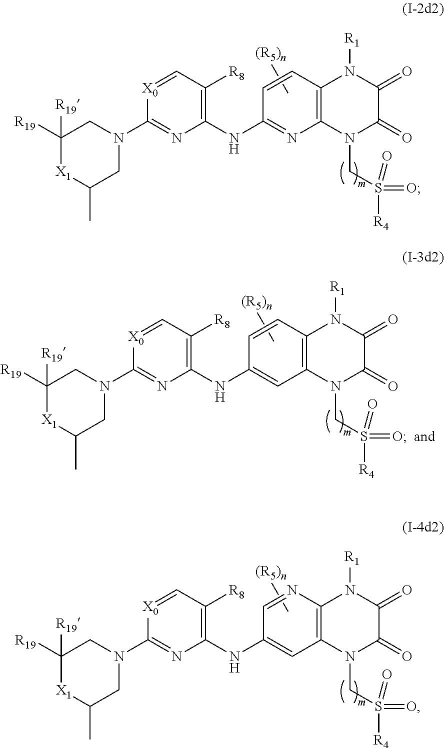

or a pharmaceutically acceptable salt or stereoisomer thereof.

In some embodiments, R₄ is optionally substituted NH—(C₁-C₆) alkyl. In some embodiments, R₄ is NH-methyl. In some embodiments, R₄ is OH.

In some embodiments, R₄ is optionally substituted NH—(C₁-C₆) hydroxyalkyl. In some embodiments R₄ is

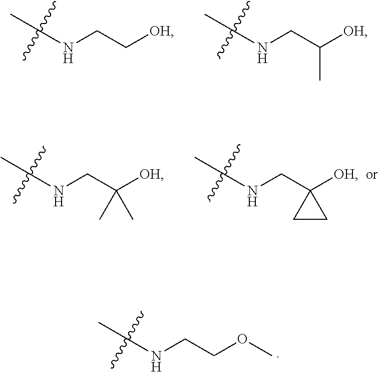

In some embodiments, R₄ is optionally substituted NH—(C₁-C₆) aminoalkyl. In some embodiments, R₄ is

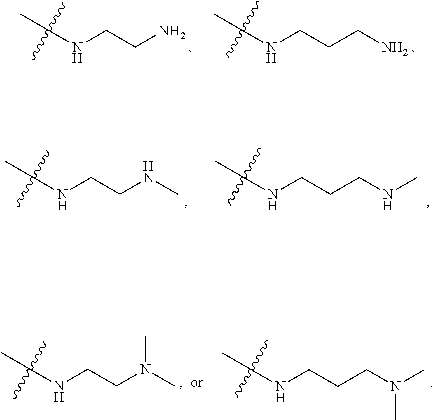

In some embodiments, R₄ is optionally substituted NH-4- to 6-membered heterocyclyl. In some embodiments, R₄ is

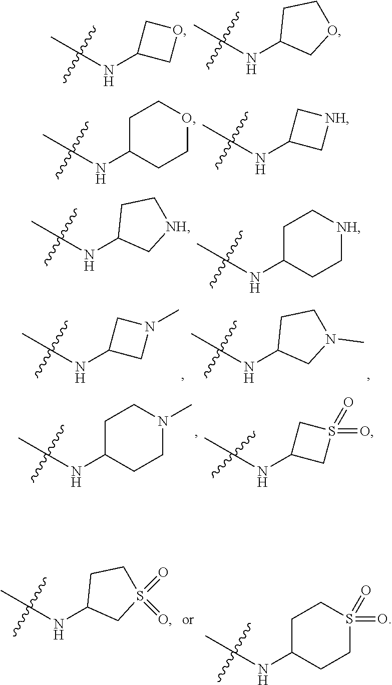

In some embodiments, R₄ is optionally substituted NH—(C₁-C₆) alkyl-4- to 6-membered heterocyclyl. In some embodiments, R₄ is

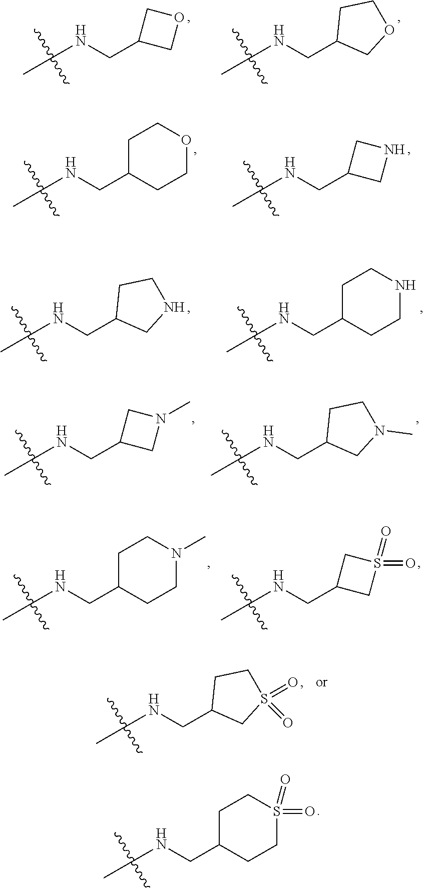

In some embodiments, n is 0 or 1.

In some embodiments, R₅ is F. In some embodiments, R₅ is methoxy.

In some embodiments, both R₆ and R_{6′} are H or F. In some embodiments, R₆ is H and R_{6′} is F.

In some embodiments, X₀ is N.

In some embodiments, the compounds of the disclosure are represented by any one of the following structures:

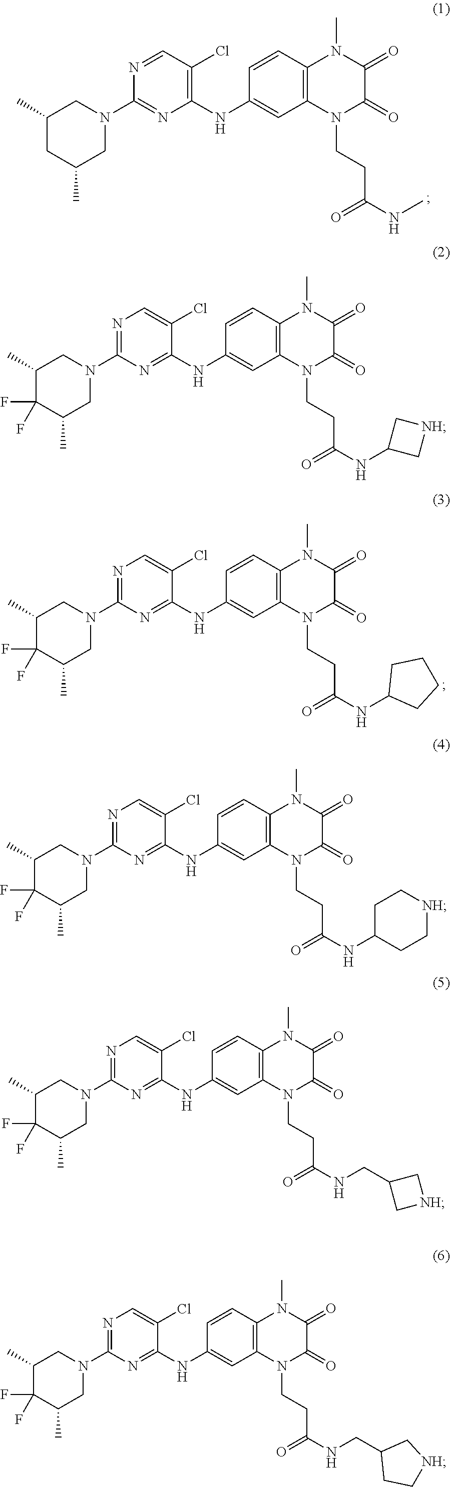

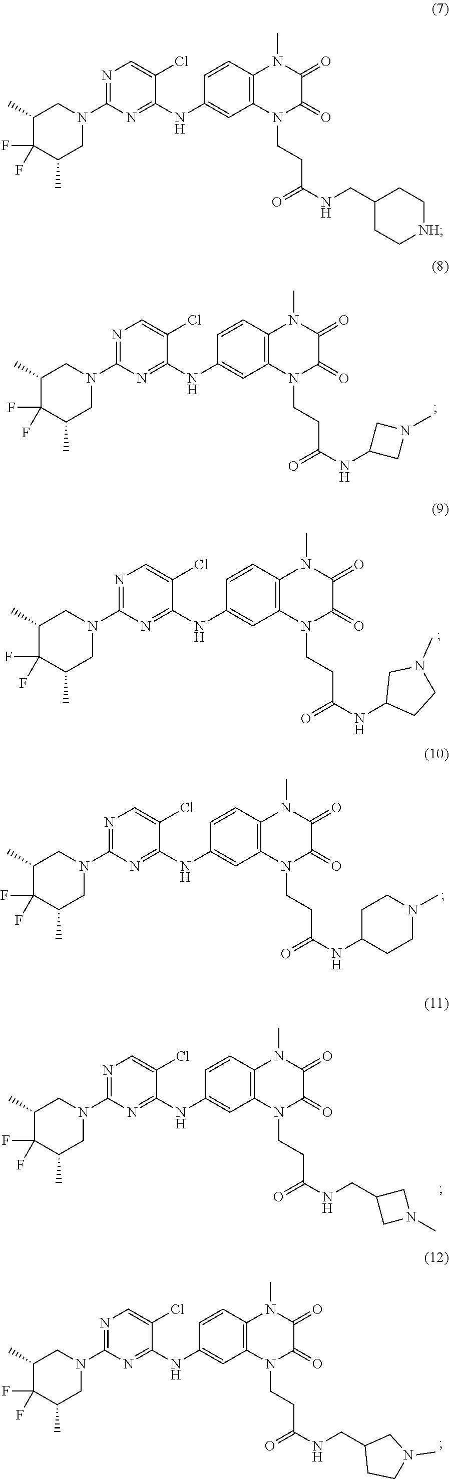

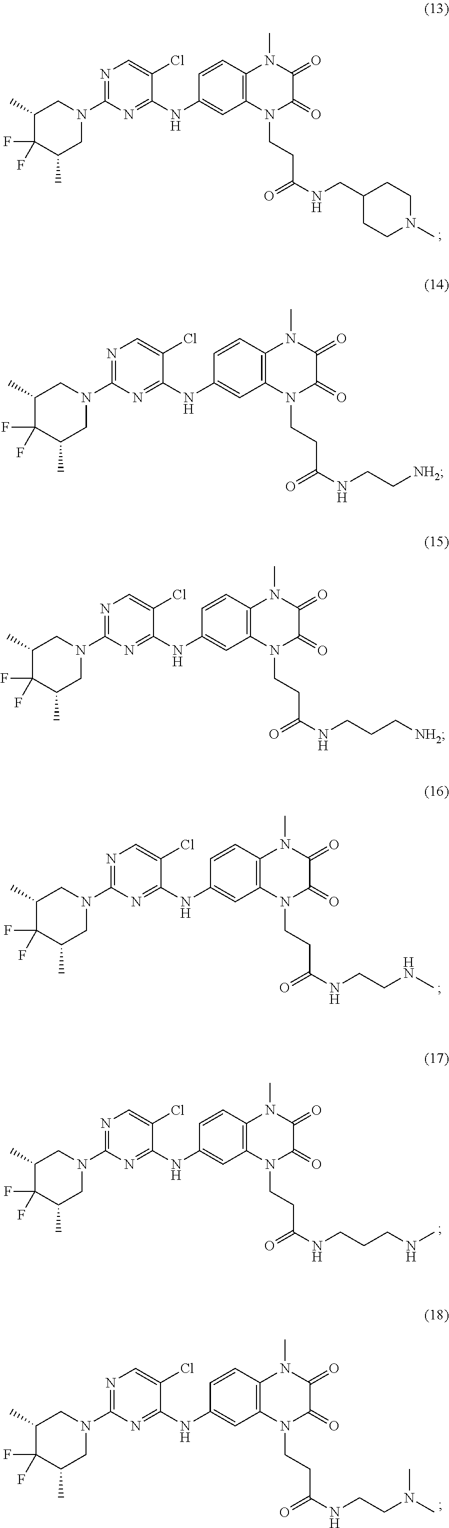

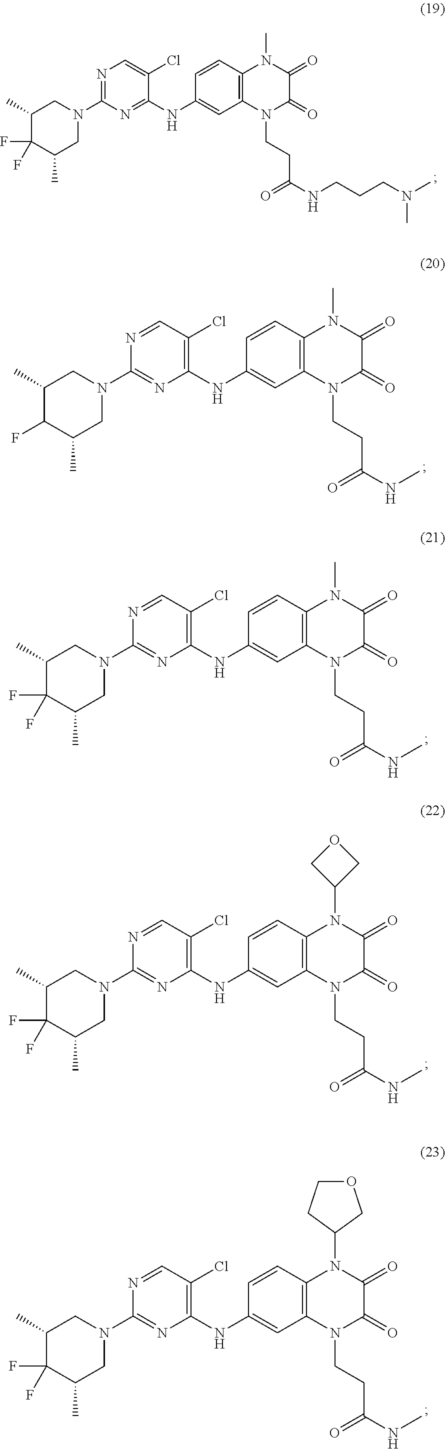

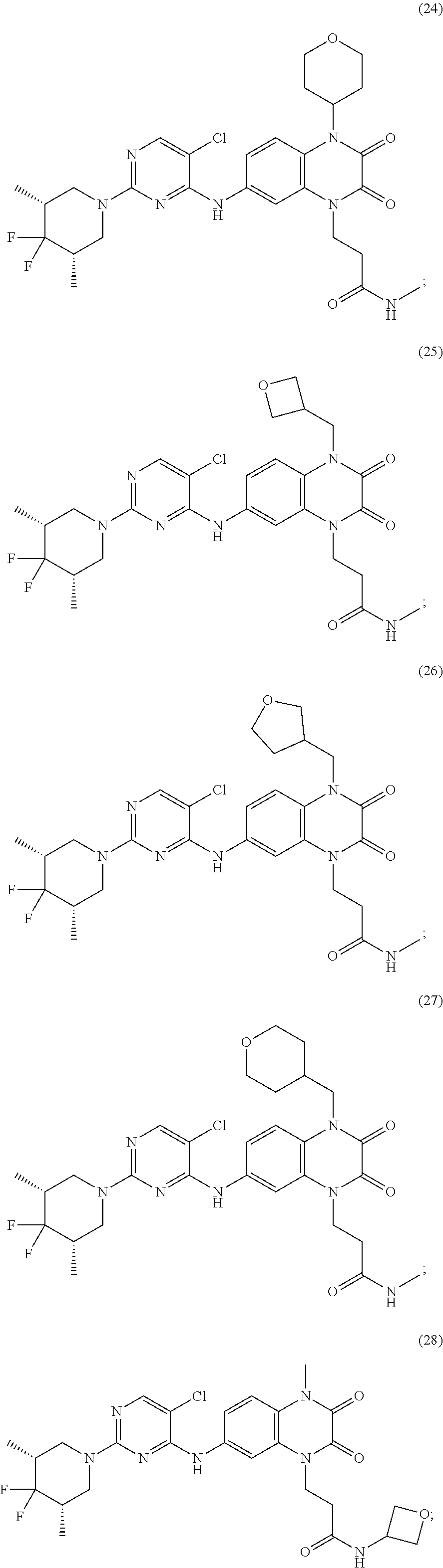 [SMILES: CNC(=O)CCn1c(=O)c(=O)n(C2CCOCC2)c2ccc(Nc3nc(N4C[C@@H](C)C(F)(F)[C@@H](C)C4)ncc3Cl)cc21 ; CNC(=O)CCn1c(=O)c(=O)n(CC2COC2)c2ccc(Nc3nc(N4C[C@@H](C)C(F)(F)[C@@H](C)C4)ncc3Cl)cc21 ; CNC(=O)CCn1c(=O)c(=O)n(CC2CCOC2)c2ccc(Nc3nc(N4C[C@@H](C)C(F)(F)[C@@H](C)C4)ncc3Cl)cc21 ; CNC(=O)CCn1c(=O)c(=O)n(CC2CCOCC2)c2ccc(Nc3nc(N4C[C@@H](C)C(F)(F)[C@@H](C)C4)ncc3Cl)cc21 ; C[C@@H]1CN(c2ncc(Cl)c(Nc3ccc4c(c3)n(CCC(=O)NC3COC3)c(=O)c(=O)n4C)n2)C[C@H](C)C1(F)F]

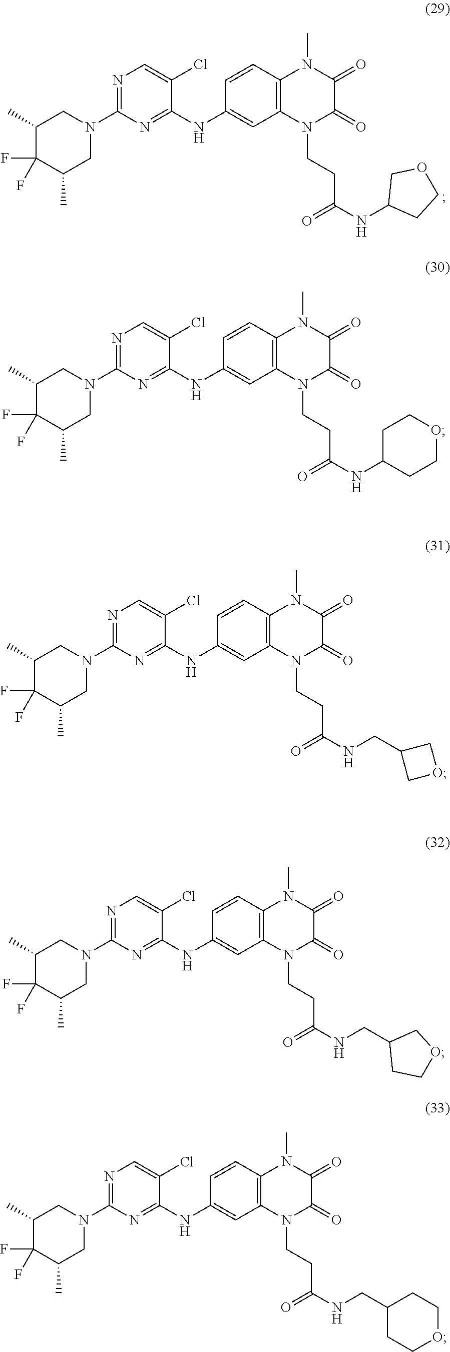 [SMILES: C[C@@H]1CN(c2ncc(Cl)c(Nc3ccc4c(c3)n(CCC(=O)NC3CCOC3)c(=O)c(=O)n4C)n2)C[C@H](C)C1(F)F ; C[C@@H]1CN(c2ncc(Cl)c(Nc3ccc4c(c3)n(CCC(=O)NC3CCOCC3)c(=O)c(=O)n4C)n2)C[C@H](C)C1(F)F ; C[C@@H]1CN(c2ncc(Cl)c(Nc3ccc4c(c3)n(CCC(=O)NCC3COC3)c(=O)c(=O)n4C)n2)C[C@H](C)C1(F)F ; C[C@@H]1CN(c2ncc(Cl)c(Nc3ccc4c(c3)n(CCC(=O)NCC3CCOC3)c(=O)c(=O)n4C)n2)C[C@H](C)C1(F)F ; C[C@@H]1CN(c2ncc(Cl)c(Nc3ccc4c(c3)n(CCC(=O)NCC3CCOCC3)c(=O)c(=O)n4C)n2)C[C@H](C)C1(F)F]

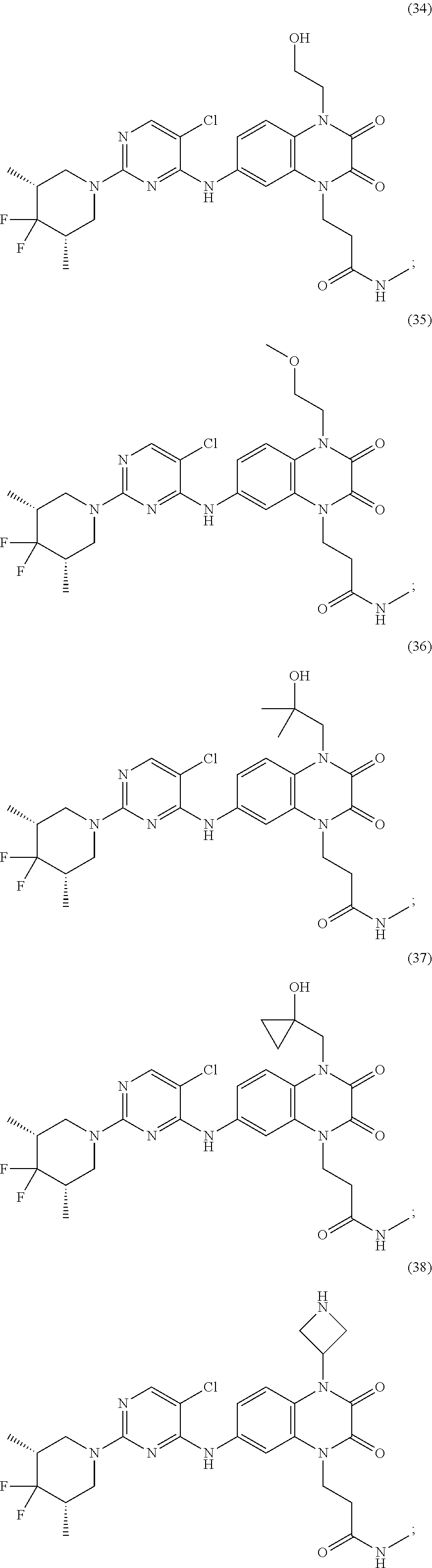 [SMILES: CNC(=O)CCn1c(=O)c(=O)n(CCO)c2ccc(Nc3nc(N4C[C@@H](C)C(F)(F)[C@@H](C)C4)ncc3Cl)cc21 ; CNC(=O)CCn1c(=O)c(=O)n(CCOC)c2ccc(Nc3nc(N4C[C@@H](C)C(F)(F)[C@@H](C)C4)ncc3Cl)cc21 ; CNC(=O)CCn1c(=O)c(=O)n(CC(C)(C)O)c2ccc(Nc3nc(N4C[C@@H](C)C(F)(F)[C@@H](C)C4)ncc3Cl)cc21 ; CNC(=O)CCn1c(=O)c(=O)n(CC2(O)CC2)c2ccc(Nc3nc(N4C[C@@H](C)C(F)(F)[C@@H](C)C4)ncc3Cl)cc21 ; CNC(=O)CCn1c(=O)c(=O)n(C2CNC2)c2ccc(Nc3nc(N4C[C@@H](C)C(F)(F)[C@@H](C)C4)ncc3Cl)cc21]

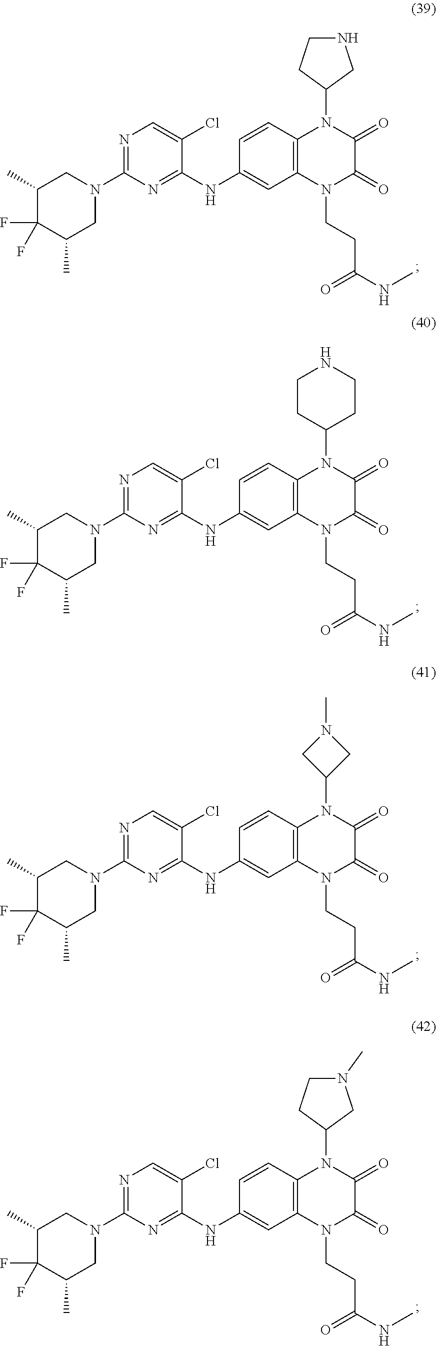 [SMILES: CNC(=O)CCn1c(=O)c(=O)n(C2CCNC2)c2ccc(Nc3nc(N4C[C@@H](C)C(F)(F)[C@@H](C)C4)ncc3Cl)cc21 ; CNC(=O)CCn1c(=O)c(=O)n(C2CCNCC2)c2ccc(Nc3nc(N4C[C@@H](C)C(F)(F)[C@@H](C)C4)ncc3Cl)cc21 ; CNC(=O)CCn1c(=O)c(=O)n(C2CN(C)C2)c2ccc(Nc3nc(N4C[C@@H](C)C(F)(F)[C@@H](C)C4)ncc3Cl)cc21 ; CNC(=O)CCn1c(=O)c(=O)n(C2CCN(C)C2)c2ccc(Nc3nc(N4C[C@@H](C)C(F)(F)[C@@H](C)C4)ncc3Cl)cc21]

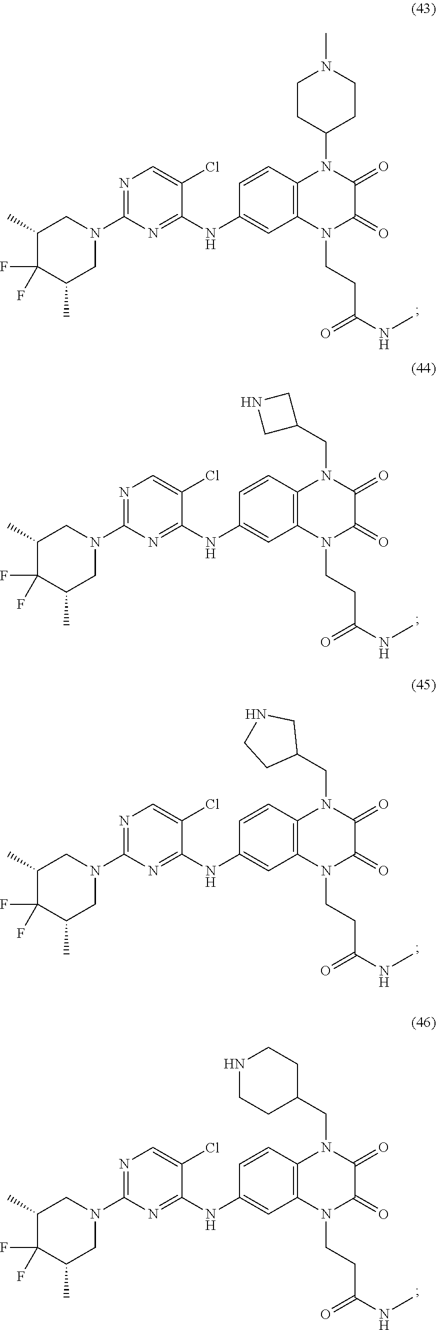 [SMILES: CNC(=O)CCn1c(=O)c(=O)n(C2CCN(C)CC2)c2ccc(Nc3nc(N4C[C@@H](C)C(F)(F)[C@@H](C)C4)ncc3Cl)cc21 ; CNC(=O)CCn1c(=O)c(=O)n(CC2CNC2)c2ccc(Nc3nc(N4C[C@@H](C)C(F)(F)[C@@H](C)C4)ncc3Cl)cc21 ; CNC(=O)CCn1c(=O)c(=O)n(CC2CCNC2)c2ccc(Nc3nc(N4C[C@@H](C)C(F)(F)[C@@H](C)C4)ncc3Cl)cc21 ; CNC(=O)CCn1c(=O)c(=O)n(CC2CCNCC2)c2ccc(Nc3nc(N4C[C@@H](C)C(F)(F)[C@@H](C)C4)ncc3Cl)cc21]

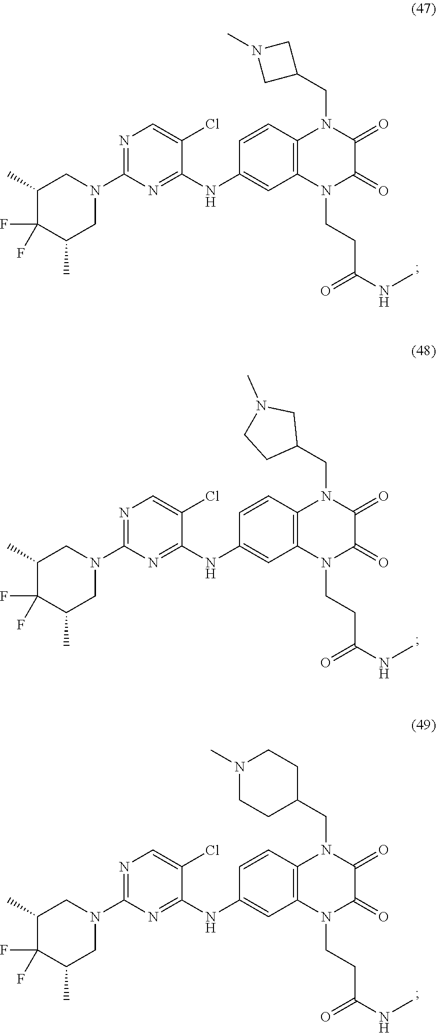 [SMILES: CNC(=O)CCn1c(=O)c(=O)n(CC2CN(C)C2)c2ccc(Nc3nc(N4C[C@@H](C)C(F)(F)[C@@H](C)C4)ncc3Cl)cc21 ; CNC(=O)CCn1c(=O)c(=O)n(CC2CCN(C)C2)c2ccc(Nc3nc(N4C[C@@H](C)C(F)(F)[C@@H](C)C4)ncc3Cl)cc21 ; CNC(=O)CCn1c(=O)c(=O)n(CC2CCN(C)CC2)c2ccc(Nc3nc(N4C[C@@H](C)C(F)(F)[C@@H](C)C4)ncc3Cl)cc21]

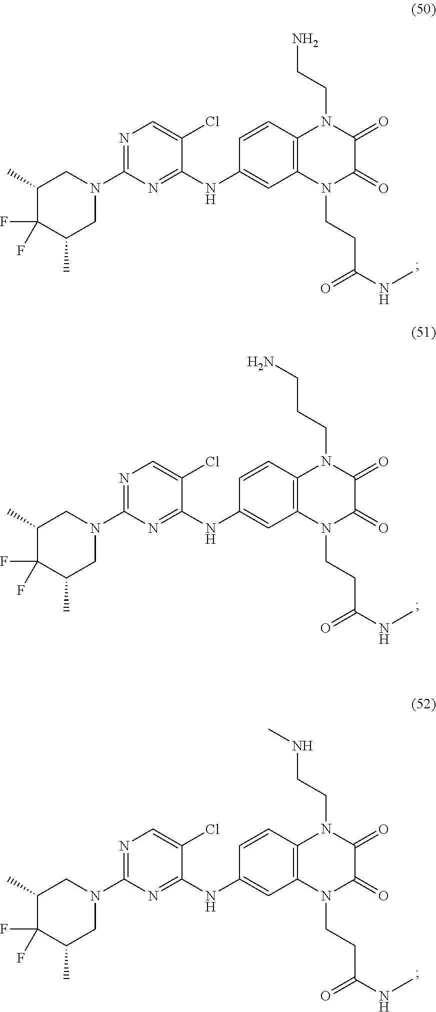 [SMILES: CNC(=O)CCn1c(=O)c(=O)n(CCN)c2ccc(Nc3nc(N4C[C@@H](C)C(F)(F)[C@@H](C)C4)ncc3Cl)cc21 ; CNC(=O)CCn1c(=O)c(=O)n(CCCN)c2ccc(Nc3nc(N4C[C@@H](C)C(F)(F)[C@@H](C)C4)ncc3Cl)cc21 ; CNCCn1c(=O)c(=O)n(CCC(=O)NC)c2cc(Nc3nc(N4C[C@@H](C)C(F)(F)[C@@H](C)C4)ncc3Cl)ccc21]

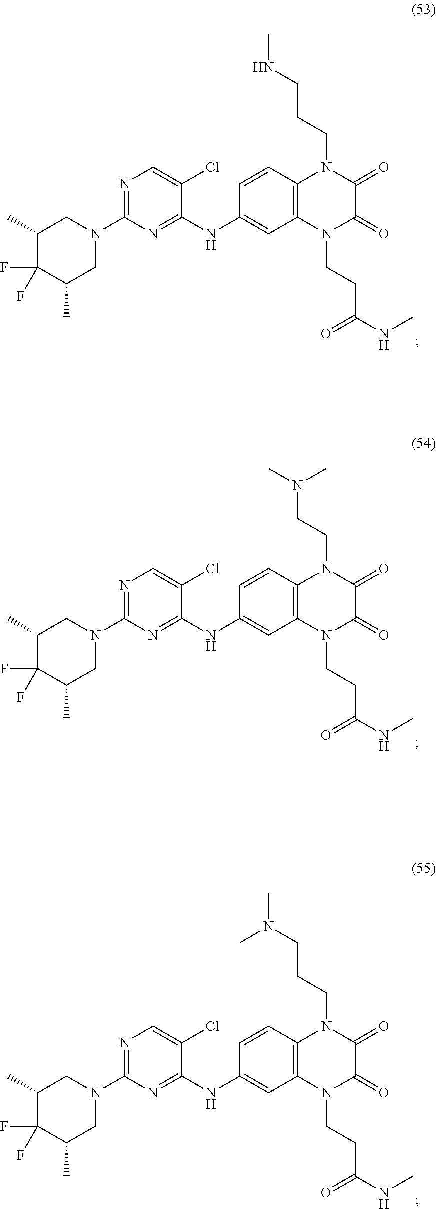 [SMILES: CNCCCn1c(=O)c(=O)n(CCC(=O)NC)c2cc(Nc3nc(N4C[C@@H](C)C(F)(F)[C@@H](C)C4)ncc3Cl)ccc21 ; CNC(=O)CCn1c(=O)c(=O)n(CCN(C)C)c2ccc(Nc3nc(N4C[C@@H](C)C(F)(F)[C@@H](C)C4)ncc3Cl)cc21 ; CNC(=O)CCn1c(=O)c(=O)n(CCCN(C)C)c2ccc(Nc3nc(N4C[C@@H](C)C(F)(F)[C@@H](C)C4)ncc3Cl)cc21]

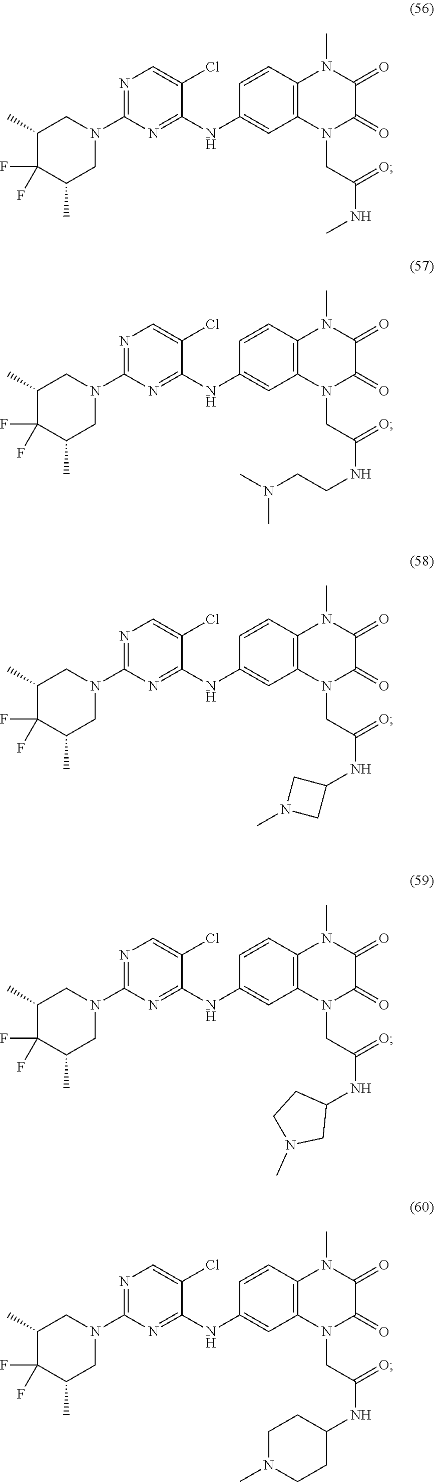 [SMILES: CNC(=O)Cn1c(=O)c(=O)n(C)c2ccc(Nc3nc(N4C[C@@H](C)C(F)(F)[C@@H](C)C4)ncc3Cl)cc21 ; C[C@@H]1CN(c2ncc(Cl)c(Nc3ccc4c(c3)n(CC(=O)NCCN(C)C)c(=O)c(=O)n4C)n2)C[C@H](C)C1(F)F ; C[C@@H]1CN(c2ncc(Cl)c(Nc3ccc4c(c3)n(CC(=O)NC3CN(C)C3)c(=O)c(=O)n4C)n2)C[C@H](C)C1(F)F ; C[C@@H]1CN(c2ncc(Cl)c(Nc3ccc4c(c3)n(CC(=O)NC3CCN(C)C3)c(=O)c(=O)n4C)n2)C[C@H](C)C1(F)F ; C[C@@H]1CN(c2ncc(Cl)c(Nc3ccc4c(c3)n(CC(=O)NC3CCN(C)CC3)c(=O)c(=O)n4C)n2)C[C@H](C)C1(F)F]

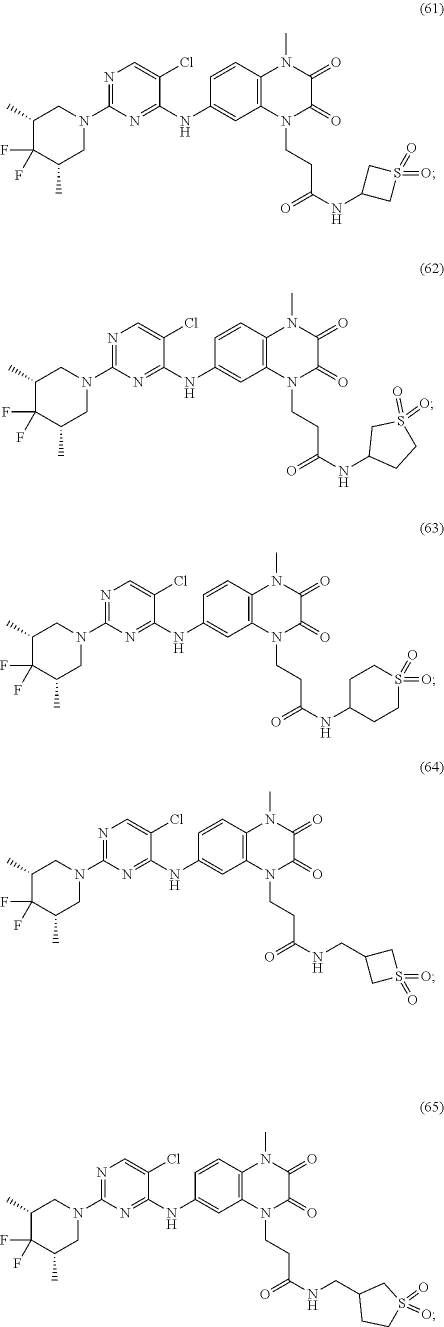 [SMILES: C[C@@H]1CN(c2ncc(Cl)c(Nc3ccc4c(c3)n(CCC(=O)NC3CS(=O)(=O)C3)c(=O)c(=O)n4C)n2)C[C@H](C)C1(F)F ; C[C@@H]1CN(c2ncc(Cl)c(Nc3ccc4c(c3)n(CCC(=O)NC3CCS(=O)(=O)C3)c(=O)c(=O)n4C)n2)C[C@H](C)C1(F)F ; C[C@@H]1CN(c2ncc(Cl)c(Nc3ccc4c(c3)n(CCC(=O)NC3CCS(=O)(=O)CC3)c(=O)c(=O)n4C)n2)C[C@H](C)C1(F)F ; C[C@@H]1CN(c2ncc(Cl)c(Nc3ccc4c(c3)n(CCC(=O)NCC3CS(=O)(=O)C3)c(=O)c(=O)n4C)n2)C[C@H](C)C1(F)F ; C[C@@H]1CN(c2ncc(Cl)c(Nc3ccc4c(c3)n(CCC(=O)NCC3CCS(=O)(=O)C3)c(=O)c(=O)n4C)n2)C[C@H](C)C1(F)F]

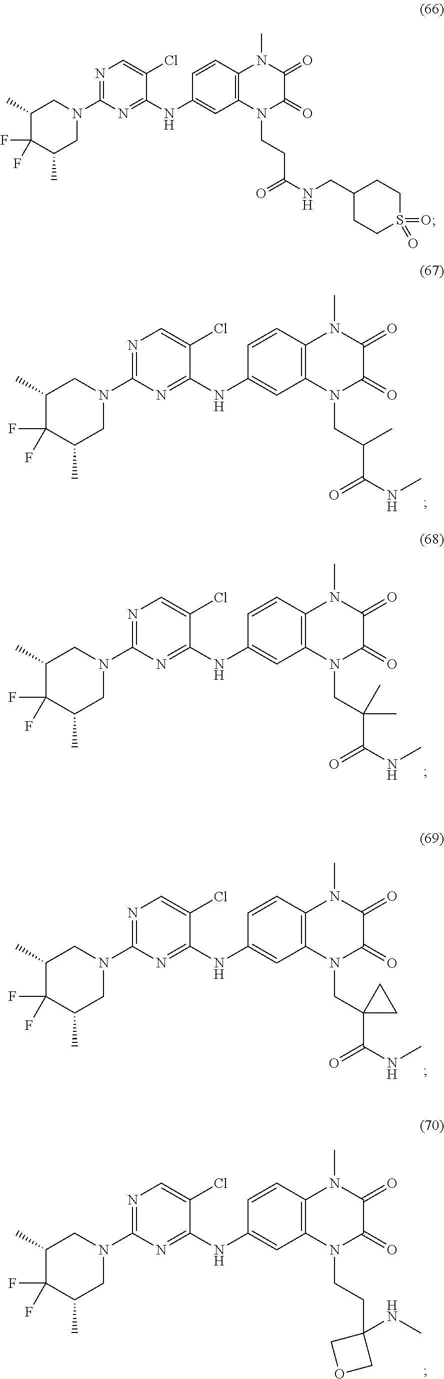 [SMILES: C[C@@H]1CN(c2ncc(Cl)c(Nc3ccc4c(c3)n(CCC(=O)NCC3CCS(=O)(=O)CC3)c(=O)c(=O)n4C)n2)C[C@H](C)C1(F)F ; CNC(=O)C(C)Cn1c(=O)c(=O)n(C)c2ccc(Nc3nc(N4C[C@@H](C)C(F)(F)[C@@H](C)C4)ncc3Cl)cc21 ; CNC(=O)C(C)(C)Cn1c(=O)c(=O)n(C)c2ccc(Nc3nc(N4C[C@@H](C)C(F)(F)[C@@H](C)C4)ncc3Cl)cc21 ; CNC(=O)C1(Cn2c(=O)c(=O)n(C)c3ccc(Nc4nc(N5C[C@@H](C)C(F)(F)[C@@H](C)C5)ncc4Cl)cc32)CC1 ; CNC1(CCn2c(=O)c(=O)n(C)c3ccc(Nc4nc(N5C[C@@H](C)C(F)(F)[C@@H](C)C5)ncc4Cl)cc32)COC1]

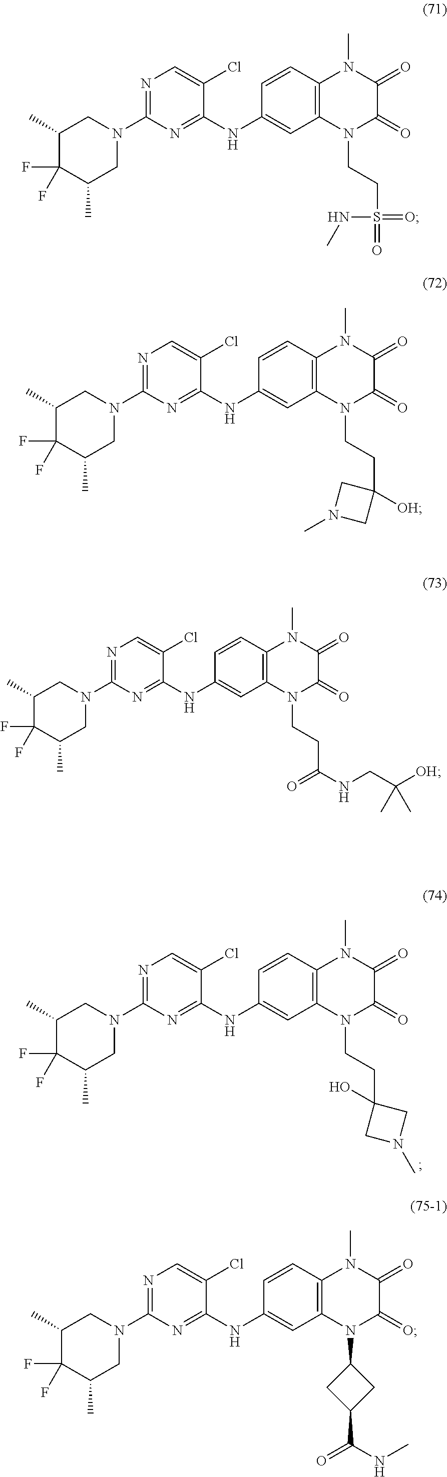 [SMILES: CNS(=O)(=O)CCn1c(=O)c(=O)n(C)c2ccc(Nc3nc(N4C[C@@H](C)C(F)(F)[C@@H](C)C4)ncc3Cl)cc21 ; C[C@@H]1CN(c2ncc(Cl)c(Nc3ccc4c(c3)n(CCC3(O)CN(C)C3)c(=O)c(=O)n4C)n2)C[C@H](C)C1(F)F ; C[C@@H]1CN(c2ncc(Cl)c(Nc3ccc4c(c3)n(CCC(=O)NCC(C)(C)O)c(=O)c(=O)n4C)n2)C[C@H](C)C1(F)F ; C[C@@H]1CN(c2ncc(Cl)c(Nc3ccc4c(c3)n(CCC3(O)CN(C)C3)c(=O)c(=O)n4C)n2)C[C@H](C)C1(F)F ; CNC(=O)[C@H]1C[C@@H](n2c(=O)c(=O)n(C)c3ccc(Nc4nc(N5C[C@@H](C)C(F)(F)[C@@H](C)C5)ncc4Cl)cc32)C1]

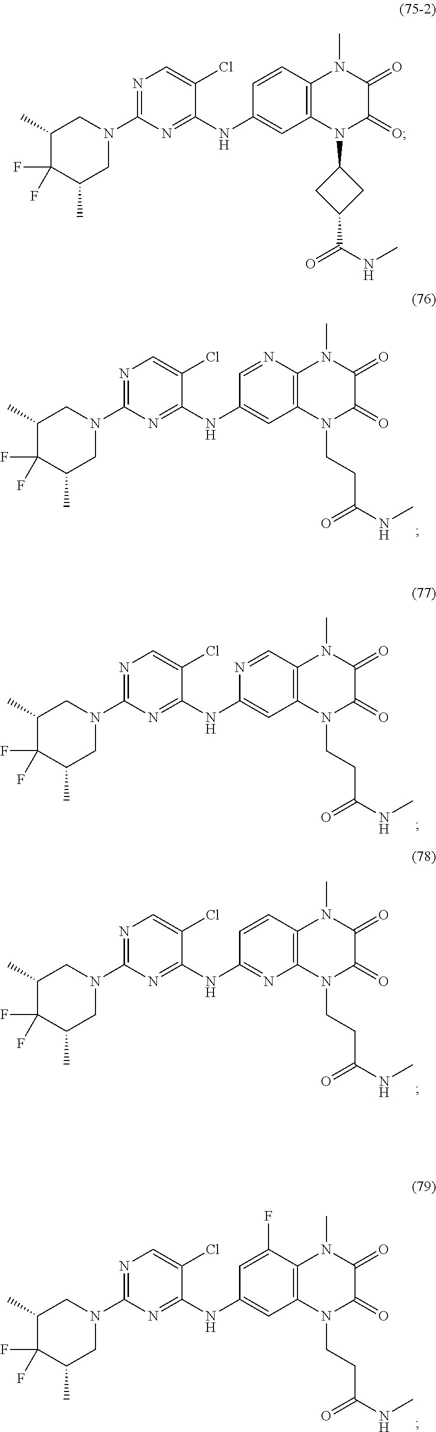 [SMILES: CNC(=O)[C@H]1C[C@H](n2c(=O)c(=O)n(C)c3ccc(Nc4nc(N5C[C@@H](C)C(F)(F)[C@@H](C)C5)ncc4Cl)cc32)C1 ; CNC(=O)CCn1c(=O)c(=O)n(C)c2ncc(Nc3nc(N4C[C@@H](C)C(F)(F)[C@@H](C)C4)ncc3Cl)cc21 ; CNC(=O)CCn1c(=O)c(=O)n(C)c2cnc(Nc3nc(N4C[C@@H](C)C(F)(F)[C@@H](C)C4)ncc3Cl)cc21 ; CNC(=O)CCn1c(=O)c(=O)n(C)c2ccc(Nc3nc(N4C[C@@H](C)C(F)(F)[C@@H](C)C4)ncc3Cl)nc21 ; CNC(=O)CCn1c(=O)c(=O)n(C)c2c(F)cc(Nc3nc(N4C[C@@H](C)C(F)(F)[C@@H](C)C4)ncc3Cl)cc21]

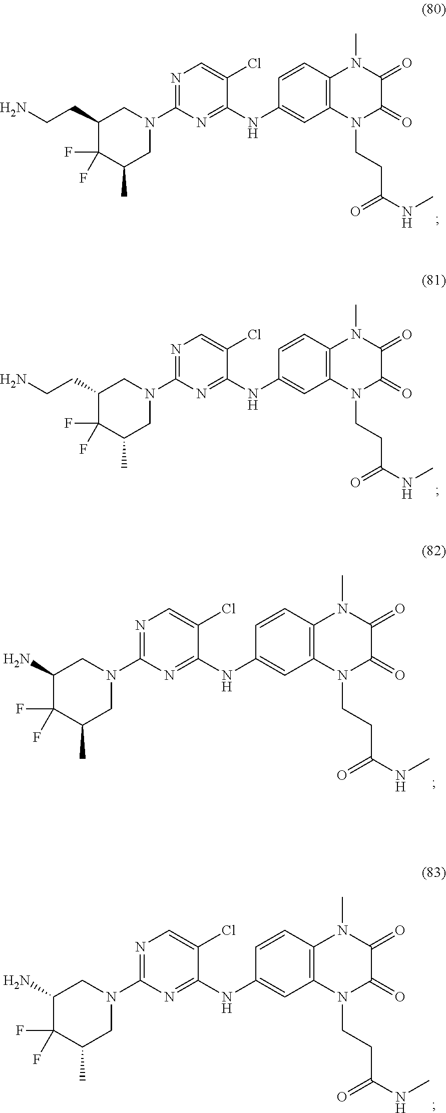 [SMILES: CNC(=O)CCn1c(=O)c(=O)n(C)c2ccc(Nc3nc(N4C[C@H](CCN)C(F)(F)[C@H](C)C4)ncc3Cl)cc21 ; CNC(=O)CCn1c(=O)c(=O)n(C)c2ccc(Nc3nc(N4C[C@@H](CCN)C(F)(F)[C@@H](C)C4)ncc3Cl)cc21 ; CNC(=O)CCn1c(=O)c(=O)n(C)c2ccc(Nc3nc(N4C[C@@H](C)C(F)(F)[C@@H](N)C4)ncc3Cl)cc21 ; CNC(=O)CCn1c(=O)c(=O)n(C)c2ccc(Nc3nc(N4C[C@@H](N)C(F)(F)[C@@H](C)C4)ncc3Cl)cc21]

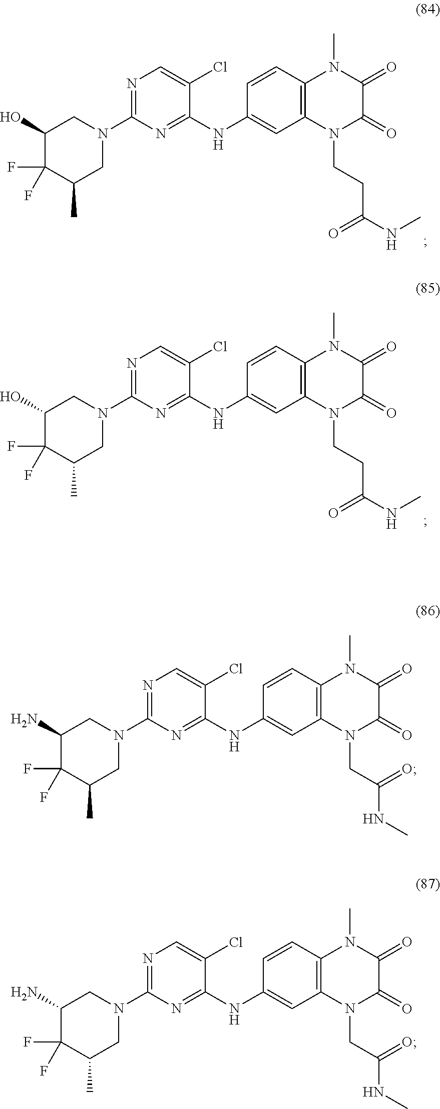 [SMILES: CNC(=O)CCn1c(=O)c(=O)n(C)c2ccc(Nc3nc(N4C[C@@H](C)C(F)(F)[C@@H](O)C4)ncc3Cl)cc21 ; CNC(=O)CCn1c(=O)c(=O)n(C)c2ccc(Nc3nc(N4C[C@@H](O)C(F)(F)[C@@H](C)C4)ncc3Cl)cc21 ; CNC(=O)Cn1c(=O)c(=O)n(C)c2ccc(Nc3nc(N4C[C@@H](C)C(F)(F)[C@@H](N)C4)ncc3Cl)cc21 ; CNC(=O)Cn1c(=O)c(=O)n(C)c2ccc(Nc3nc(N4C[C@@H](N)C(F)(F)[C@@H](C)C4)ncc3Cl)cc21]

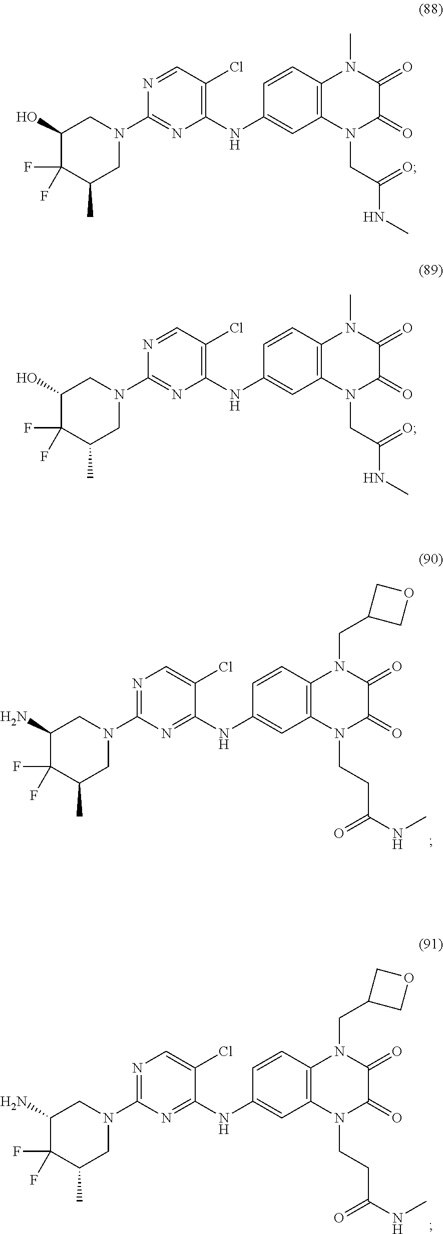 [SMILES: CNC(=O)Cn1c(=O)c(=O)n(C)c2ccc(Nc3nc(N4C[C@@H](C)C(F)(F)[C@@H](O)C4)ncc3Cl)cc21 ; CNC(=O)Cn1c(=O)c(=O)n(C)c2ccc(Nc3nc(N4C[C@@H](O)C(F)(F)[C@@H](C)C4)ncc3Cl)cc21 ; CNC(=O)CCn1c(=O)c(=O)n(CC2COC2)c2ccc(Nc3nc(N4C[C@@H](C)C(F)(F)[C@@H](N)C4)ncc3Cl)cc21 ; CNC(=O)CCn1c(=O)c(=O)n(CC2COC2)c2ccc(Nc3nc(N4C[C@@H](N)C(F)(F)[C@@H](C)C4)ncc3Cl)cc21]

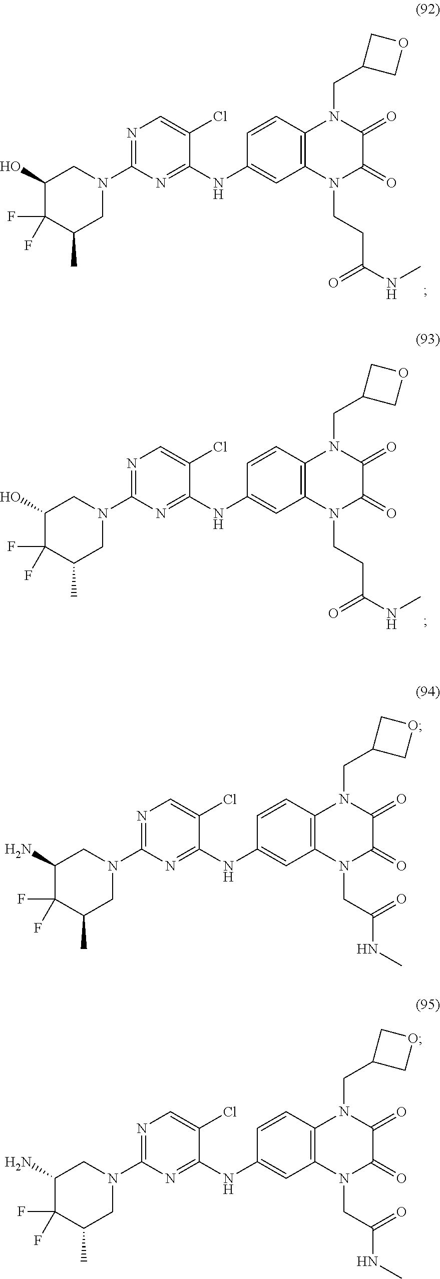 [SMILES: CNC(=O)CCn1c(=O)c(=O)n(CC2COC2)c2ccc(Nc3nc(N4C[C@@H](C)C(F)(F)[C@@H](O)C4)ncc3Cl)cc21 ; CNC(=O)CCn1c(=O)c(=O)n(CC2COC2)c2ccc(Nc3nc(N4C[C@@H](O)C(F)(F)[C@@H](C)C4)ncc3Cl)cc21 ; CNC(=O)Cn1c(=O)c(=O)n(CC2COC2)c2ccc(Nc3nc(N4C[C@@H](C)C(F)(F)[C@@H](N)C4)ncc3Cl)cc21 ; CNC(=O)Cn1c(=O)c(=O)n(CC2COC2)c2ccc(Nc3nc(N4C[C@@H](N)C(F)(F)[C@@H](C)C4)ncc3Cl)cc21]

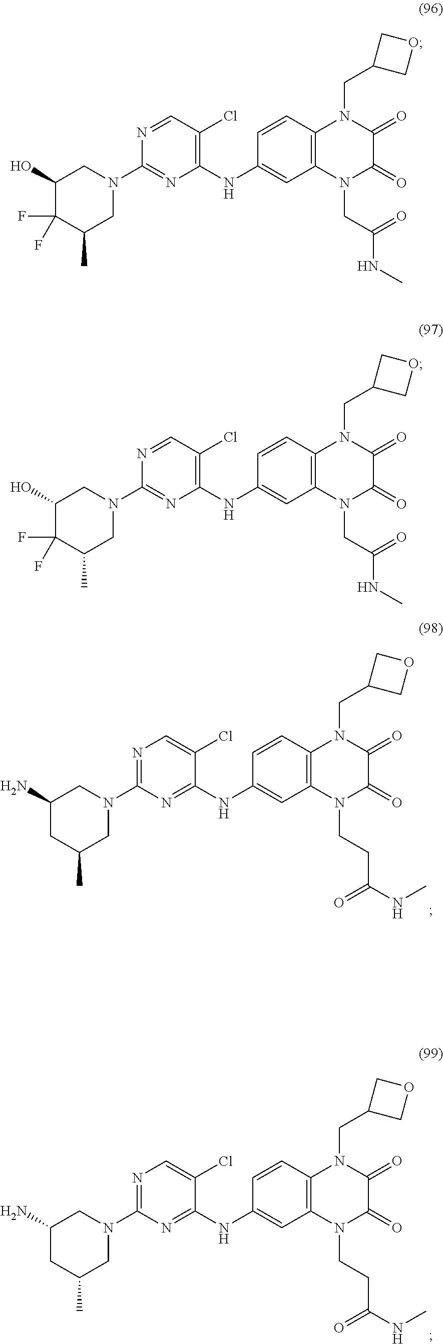 [SMILES: CNC(=O)Cn1c(=O)c(=O)n(CC2COC2)c2ccc(Nc3nc(N4C[C@@H](C)C(F)(F)[C@@H](O)C4)ncc3Cl)cc21 ; CNC(=O)Cn1c(=O)c(=O)n(CC2COC2)c2ccc(Nc3nc(N4C[C@@H](O)C(F)(F)[C@@H](C)C4)ncc3Cl)cc21 ; CNC(=O)CCn1c(=O)c(=O)n(CC2COC2)c2ccc(Nc3nc(N4C[C@@H](C)C[C@@H](N)C4)ncc3Cl)cc21 ; CNC(=O)CCn1c(=O)c(=O)n(CC2COC2)c2ccc(Nc3nc(N4C[C@H](C)C[C@H](N)C4)ncc3Cl)cc21]

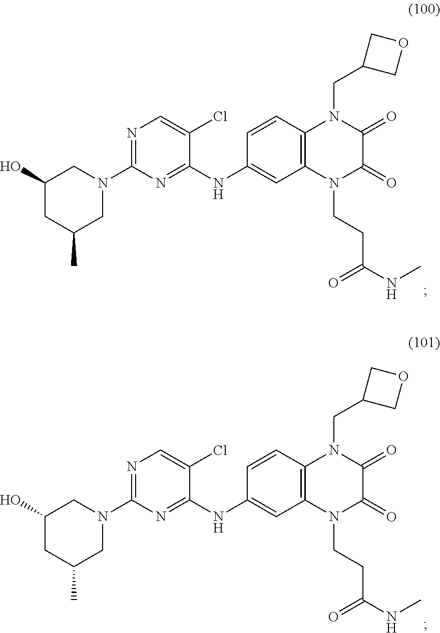 [SMILES: CNC(=O)CCn1c(=O)c(=O)n(CC2COC2)c2ccc(Nc3nc(N4C[C@@H](C)C[C@@H](O)C4)ncc3Cl)cc21 ; CNC(=O)CCn1c(=O)c(=O)n(CC2COC2)c2ccc(Nc3nc(N4C[C@H](C)C[C@H](O)C4)ncc3Cl)cc21]

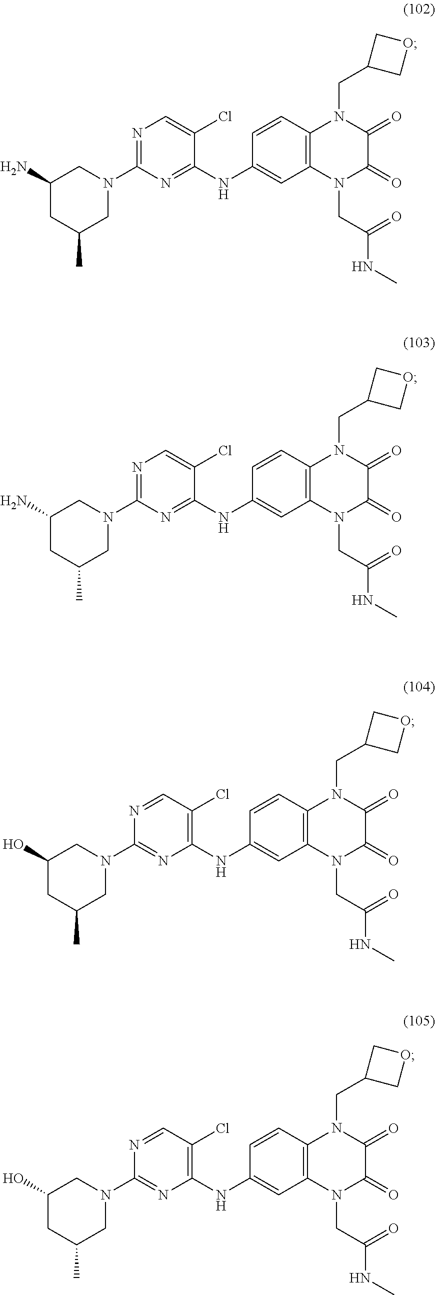 [SMILES: CNC(=O)Cn1c(=O)c(=O)n(CC2COC2)c2ccc(Nc3nc(N4C[C@@H](C)C[C@@H](N)C4)ncc3Cl)cc21 ; CNC(=O)Cn1c(=O)c(=O)n(CC2COC2)c2ccc(Nc3nc(N4C[C@H](C)C[C@H](N)C4)ncc3Cl)cc21 ; CNC(=O)Cn1c(=O)c(=O)n(CC2COC2)c2ccc(Nc3nc(N4C[C@@H](C)C[C@@H](O)C4)ncc3Cl)cc21 ; CNC(=O)Cn1c(=O)c(=O)n(CC2COC2)c2ccc(Nc3nc(N4C[C@H](C)C[C@H](O)C4)ncc3Cl)cc21]

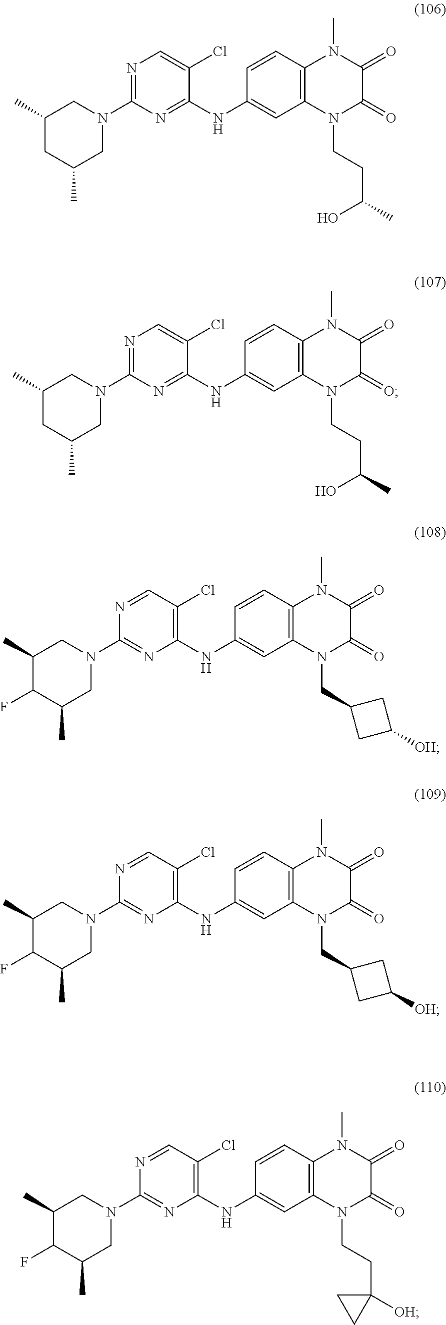

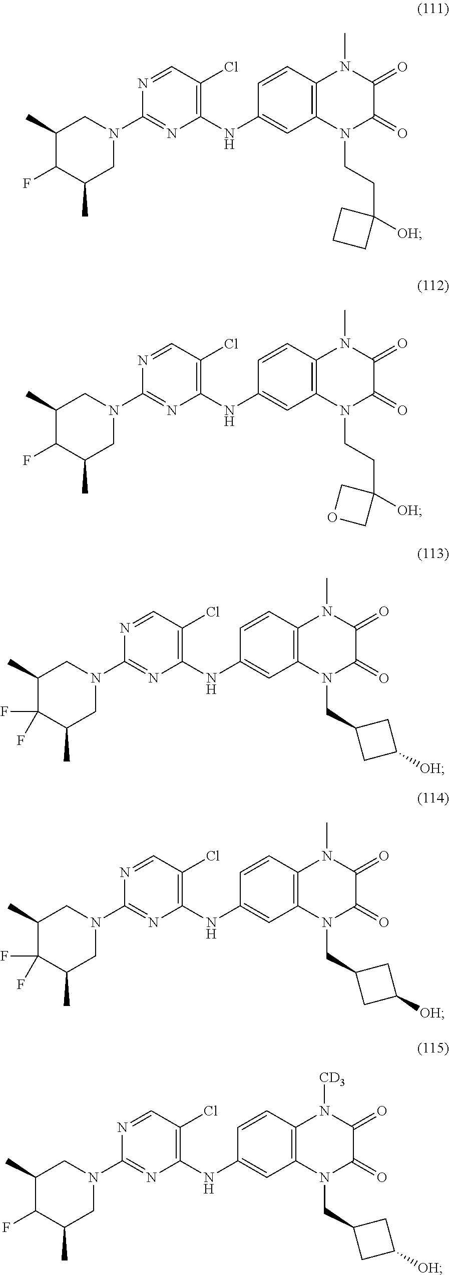 [SMILES: C[C@@H]1CN(c2ncc(Cl)c(Nc3ccc4c(c3)n(CCC3(O)CCC3)c(=O)c(=O)n4C)n2)C[C@H](C)C1F ; C[C@@H]1CN(c2ncc(Cl)c(Nc3ccc4c(c3)n(CCC3(O)COC3)c(=O)c(=O)n4C)n2)C[C@H](C)C1F ; C[C@@H]1CN(c2ncc(Cl)c(Nc3ccc4c(c3)n(C[C@H]3C[C@H](O)C3)c(=O)c(=O)n4C)n2)C[C@H](C)C1(F)F ; C[C@@H]1CN(c2ncc(Cl)c(Nc3ccc4c(c3)n(C[C@H]3C[C@@H](O)C3)c(=O)c(=O)n4C)n2)C[C@H](C)C1(F)F ; [2H]C([2H])([2H])n1c(=O)c(=O)n(C[C@H]2C[C@H](O)C2)c2cc(Nc3nc(N4C[C@@H](C)C(F)[C@@H](C)C4)ncc3Cl)ccc21]

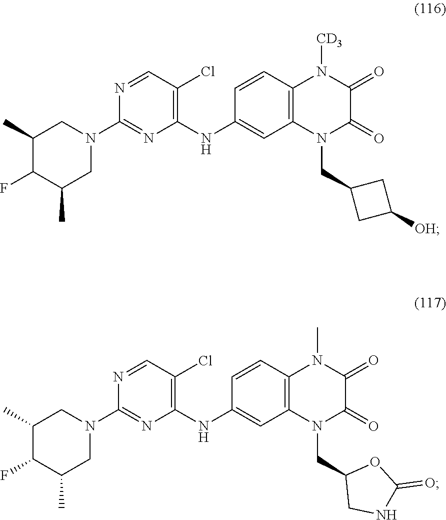 [SMILES: [2H]C([2H])([2H])n1c(=O)c(=O)n(C[C@H]2C[C@@H](O)C2)c2cc(Nc3nc(N4C[C@@H](C)C(F)[C@@H](C)C4)ncc3Cl)ccc21 ; C[C@@H]1CN(c2ncc(Cl)c(Nc3ccc4c(c3)n(C[C@@H]3CNC(=O)O3)c(=O)c(=O)n4C)n2)C[C@H](C)[C@@H]1F]

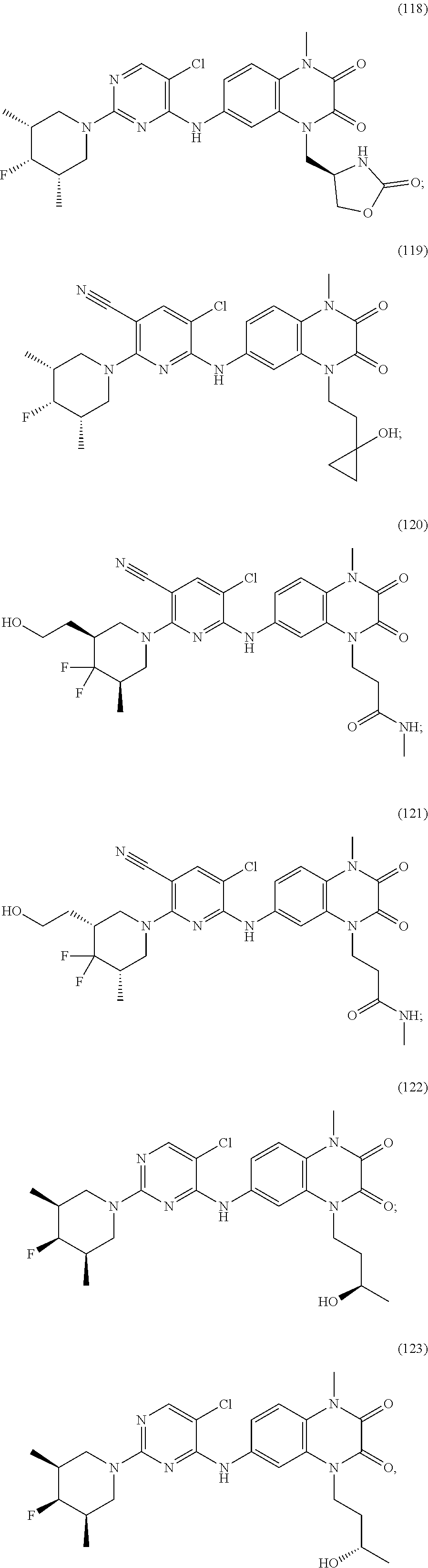

or a pharmaceutically acceptable salt or stereoisomer thereof.

Compounds of the present disclosure may be in the form of a free acid or free base, or a pharmaceutically acceptable salt. A pharmaceutically acceptable salt of the compounds of this disclosure can be formed, for example, by reaction of an appropriate free base of a compound of the invention and an appropriate pharmaceutically acceptable acid in a suitable solvent under standard conditions well known in the art. See, for example, Gould, P. L., “Salt selection for basic drugs,” International Journal of Pharmaceutics, 33: 201-217 (1986); Bastin, R. J., et al., “Salt Selection and Optimization Procedures for Pharmaceutical New Chemical Entities,” Organic Process Research and Development, 4:427-435 (2000); and Berge, S. M., et al., “Pharmaceutical Salts,” Journal of Pharmaceutical Sciences, 66:1-19, (1977).

Compounds of the present disclosure may have at least one chiral center and thus may be in the form of a stereoisomer, which as used herein, embraces all isomers of individual compounds that differ only in the orientation of their atoms in space. The term stereoisomer includes mirror image isomers (enantiomers which include the (R-) or (S-) configurations of the compounds), mixtures of mirror image isomers (physical mixtures of the enantiomers, and racemates or racemic mixtures) of compounds, geometric (cis/trans or E/Z, R/S) isomers of compounds and isomers of compounds with more than one chiral center that are not mirror images of one another (diastereoisomers). The chiral centers of the compounds may undergo epimerization in vivo; thus, for these compounds, administration of the compound in its (R-) form is considered equivalent to administration of the compound in its (S-) form. Accordingly, the compounds of the present disclosure may be made and used in the form of individual isomers and substantially free of other isomers, or in the form of a mixture of various isomers, e.g., racemic mixtures of stereoisomers.

In some embodiments, the compound of formula (I) is an isotopic derivative in that it has at least one desired isotopic substitution of an atom, at an amount above the natural abundance of the isotope, i.e., enriched. In one embodiment, the compound includes deuterium or multiple deuterium atoms. As used herein, the term “compound” embraces isotopic derivatives.

Compounds of formula (I) may also be in the form of N-oxides, crystalline forms (also known as polymorphs), co-crystals, active metabolites of the compounds having the same type of activity, prodrugs, tautomers, and unsolvated as well as solvated (e.g., hydrated) forms with pharmaceutically acceptable solvents such as water, ethanol, and the like, of the compounds. As used herein, the term “compound” embraces all these forms.

The compounds of formula (I) may be prepared by crystallization under different conditions and may exist as one or a combination of polymorphs of the compound. For example, different polymorphs may be identified and/or prepared using different solvents, or different mixtures of solvents for recrystallization, by performing crystallizations at different temperatures, or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other known techniques.

In some embodiments, the pharmaceutical composition comprises a co-crystal of a compound of formula (I). The term “co-crystal”, as used herein, refers to a stoichiometric multi-component system comprising a compound of formula (I) and a co-crystal former wherein the compound of formula (I) and the co-crystal former are connected by non-covalent interactions. The term “co-crystal former”, as used herein, refers to compounds which can form intermolecular interactions with a compound of formula (I) and co-crystallize with it. Representative examples of co-crystal formers include benzoic acid, succinic acid, fumaric acid, glutaric acid, trans-cinnamic acid, 2,5-dihvdroxybenzoic acid, glycolic acid, trans-2-hexanoic acid, 2-hydroxycaproic acid, lactic acid, sorbic acid, tartaric acid, ferulic acid, suberic acid, picolinic acid, salicylic acid, maleic acid, saccharin, 4,4′-bipyridine p-aminosalicylic acid, nicotinamide, urea, isonicotinamide, methyl-4-hydroxybenzoate, adipic acid, terephthalic acid, resorcinol, pyrogallol, phloroglucinol, hydroxyquinol, isoniazid, theophylline, adenine, theobromine, phenacetin, phenazone, etofylline, and phenobarbital.

### Methods of Synthesis

In another aspect, the present disclosure is directed to a method for making a compound of formula (I), or a pharmaceutically acceptable salt or stereoisomer thereof. Broadly, the compounds of formula (I) or pharmaceutically-acceptable salts or stereoisomers thereof may be prepared by any process known to be applicable to the preparation of chemically related compounds. The compounds of formula (I) will be better understood in connection with the synthetic schemes that described in various working examples and which illustrate nonlimiting methods by which the compounds of formula (I) may be prepared.

### Pharmaceutical Compositions

Another aspect of the present disclosure is directed to a pharmaceutical composition that includes a therapeutically effective amount of a compound of formula (I) or a pharmaceutically acceptable salt or stereoisomer thereof, and a pharmaceutically acceptable carrier. The term “pharmaceutically acceptable carrier,” as known in the art, refers to a pharmaceutically acceptable material, composition or vehicle, suitable for administering compounds of the present disclosure to mammals. Suitable carriers may include, for example, liquids (both aqueous and non-aqueous alike, and combinations thereof), solids, encapsulating materials, gases, and combinations thereof (e.g., semi-solids), and gases, that function to carry or transport the compound from one organ, or portion of the body, to another organ, or portion of the body. A carrier is “acceptable” in the sense of being physiologically inert to and compatible with the other ingredients of the formulation and not injurious to the subject or patient. Depending on the type of formulation, the composition may also include one or more pharmaceutically acceptable excipients.

Broadly, compounds of formula (I) and their pharmaceutically acceptable salts and stereoisomers may be formulated into a given type of composition in accordance with conventional pharmaceutical practice such as conventional mixing, dissolving, granulating, dragee-making, levigating, emulsifying, encapsulating, entrapping and compression processes (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). The type of formulation depends on the mode of administration which may include enteral (e.g., oral, buccal, sublingual and rectal), parenteral (e.g., subcutaneous (s.c.), intravenous (i.v.), intramuscular (i.m.), and intrasternal injection, or infusion techniques, intra-ocular, intra-arterial, intramedullary, intrathecal, intraventricular, transdermal, interdermal, intravaginal, intraperitoneal, mucosal, nasal, intratracheal instillation, bronchial instillation, and inhalation) and topical (e.g., transdermal). In general, the most appropriate route of administration will depend upon a variety of factors including, for example, the nature of the agent (e.g., its stability in the environment of the gastrointestinal tract), and/or the condition of the subject (e.g., whether the subject is able to tolerate oral administration). For example, parenteral (e.g., intravenous) administration may also be advantageous in that the compound may be administered relatively quickly such as in the case of a single-dose treatment and/or an acute condition.

In some embodiments, the compounds of formula (I) are formulated for oral or intravenous administration (e.g., systemic intravenous injection).

Accordingly, compounds of formula (I) may be formulated into solid compositions (e.g., powders, tablets, dispersible granules, capsules, cachets, and suppositories), liquid compositions (e.g., solutions in which the compound is dissolved, suspensions in which solid particles of the compound are dispersed, emulsions, and solutions containing liposomes, micelles, or nanoparticles, syrups and elixirs); semi-solid compositions (e.g., gels, suspensions and creams); and gases (e.g., propellants for aerosol compositions). Compounds may also be formulated for rapid, intermediate or extended release.

Solid dosage forms for oral administration include capsules, tablets, pills, powders, and granules. In such solid dosage forms, the active compound is mixed with a carrier such as sodium citrate or dicalcium phosphate and an additional carrier or excipient such as a) fillers or extenders such as starches, lactose, sucrose, glucose, mannitol, and silicic acid, b) binders such as, for example, methylcellulose, microcrystalline cellulose, hydroxypropylmethylcellulose, carboxymethylcellulose, sodium carboxymethylcellulose, alginates, gelatin, polyvinylpyrrolidinone, sucrose, and acacia, c) humectants such as glycerol, d) disintegrating agents such as crosslinked polymers (e.g., crosslinked polyvinylpyrrolidone (crospovidone), crosslinked sodium carboxymethyl cellulose (croscarmellose sodium), sodium starch glycolate, agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also include buffering agents. Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings. They may further contain an opacifying agent.

In some embodiments, compounds of formula (I) may be formulated in a hard or soft gelatin capsule. Representative excipients that may be used include pregelatinized starch, magnesium stearate, mannitol, sodium stearyl fumarate, lactose anhydrous, microcrystalline cellulose and croscarmellose sodium. Gelatin shells may include gelatin, titanium dioxide, iron oxides and colorants.

Liquid dosage forms for oral administration include solutions, suspensions, emulsions, micro-emulsions, syrups and elixirs. In addition to the compound, the liquid dosage forms may contain an aqueous or non-aqueous carrier (depending upon the solubility of the compounds) commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Oral compositions may also include an excipients such as wetting agents, suspending agents, coloring, sweetening, flavoring, and perfuming agents.

Injectable preparations for parenteral administration may include sterile aqueous solutions or oleaginous suspensions. They may be formulated according to standard techniques using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables. The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use. The effect of the compound may be prolonged by slowing its absorption, which may be accomplished by the use of a liquid suspension or crystalline or amorphous material with poor water solubility. Prolonged absorption of the compound from a parenterally administered formulation may also be accomplished by suspending the compound in an oily vehicle.

In certain embodiments, compounds of formula (I) may be administered in a local rather than systemic manner, for example, via injection of the conjugate directly into an organ, often in a depot preparation or sustained release formulation. In specific embodiments, long-acting formulations are administered by implantation (for example subcutaneously or intramuscularly) or by intramuscular injection. Injectable depot forms are made by forming microencapsule matrices of the compound in a biodegradable polymer, e.g., polylactide-polyglycolides, poly(orthoesters) and poly(anhydrides). The rate of release of the compound may be controlled by varying the ratio of compound to polymer and the nature of the particular polymer employed. Depot injectable formulations are also prepared by entrapping the compound in liposomes or microemulsions that are compatible with body tissues. Furthermore, in other embodiments, the compound is delivered in a targeted drug delivery system, for example, in a liposome coated with organ-specific antibody. In such embodiments, the liposomes are targeted to and taken up selectively by the organ.

The compositions may be formulated for buccal or sublingual administration, examples of which include tablets, lozenges and gels.

The compounds of formula (I) may be formulated for administration by inhalation. Various forms suitable for administration by inhalation include aerosols, mists or powders. Pharmaceutical compositions may be delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In some embodiments, the dosage unit of a pressurized aerosol may be determined by providing a valve to deliver a metered amount. In some embodiments, capsules and cartridges including gelatin, for example, for use in an inhaler or insufflator, may be formulated containing a powder mix of the compound and a suitable powder base such as lactose or starch.

Compounds of formula (I) may be formulated for topical administration which as used herein, refers to administration intradermally of the formulation to the epidermis. These types of compositions are typically in the form of ointments, pastes, creams, lotions, gels, solutions and sprays.

Representative examples of carriers useful in formulating compounds for topical application include solvents (e.g., alcohols, poly alcohols, water), creams, lotions, ointments, oils, plasters, liposomes, powders, emulsions, microemulsions, and buffered solutions (e.g., hypotonic or buffered saline). Creams, for example, may be formulated using saturated or unsaturated fatty acids such as stearic acid, palmitic acid, oleic acid, palmito-oleic acid, cetyl, or oleyl alcohols. Creams may also contain a non-ionic surfactant such as polyoxy-40-stearate.

In some embodiments, the topical formulations may also include an excipient, an example of which is a penetration enhancing agent. These agents are capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). Representative examples of penetration enhancing agents include triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate), and N-methylpyrrolidone.

Representative examples of yet other excipients that may be included in topical as well as in other types of formulations (to the extent they are compatible), include preservatives, antioxidants, moisturizers, emollients, buffering agents, solubilizing agents, skin protectants, and surfactants. Suitable preservatives include alcohols, quaternary amines, organic acids, parabens, and phenols. Suitable antioxidants include ascorbic acid and its esters, sodium bisulfite, butylated hydroxytoluene, butylated hydroxyanisole, tocopherols, and chelating agents like EDTA and citric acid. Suitable moisturizers include glycerin, sorbitol, polyethylene glycols, urea, and propylene glycol. Suitable buffering agents include citric, hydrochloric, and lactic acid buffers. Suitable solubilizing agents include quaternary ammonium chlorides, cyclodextrins, benzyl benzoate, lecithin, and polysorbates. Suitable skin protectants include vitamin E oil, allatoin, dimethicone, glycerin, petrolatum, and zinc oxide.

Transdermal formulations typically employ transdermal delivery devices and transdermal delivery patches wherein the compound is formulated in lipophilic emulsions or buffered, aqueous solutions, dissolved and/or dispersed in a polymer or an adhesive. Patches may be constructed for continuous, pulsatile, or on demand delivery of pharmaceutical agents. Transdermal delivery of the compounds may be accomplished by means of an iontophoretic patch. Transdermal patches may provide controlled delivery of the compounds wherein the rate of absorption is slowed by using rate-controlling membranes or by trapping the compound within a polymer matrix or gel. Absorption enhancers may be used to increase absorption, examples of which include absorbable pharmaceutically acceptable solvents that assist passage through the skin.

Ophthalmic formulations include eye drops.

Formulations for rectal administration include enemas, rectal gels, rectal foams, rectal aerosols, and retention enemas, which may contain conventional suppository bases such as cocoa butter or other glycerides, as well as synthetic polymers such as polyvinylpyrrolidone, PEG, and the like. Compositions for rectal or vaginal administration may also be formulated as suppositories which can be prepared by mixing the compound with suitable non-irritating carriers and excipients such as cocoa butter, mixtures of fatty acid glycerides, polyethylene glycol, suppository waxes, and combinations thereof, all of which are solid at ambient temperature but liquid at body temperature and therefore melt in the rectum or vaginal cavity and release the compound.

### Dosage Amounts

As used herein, the term, “therapeutically effective amount” refers to an amount of a compound of formula (I) or a pharmaceutically acceptable salt or a stereoisomer thereof that is effective in producing the desired therapeutic response in a particular patient suffering from a disease or disorder mediated by aberrant BCL6 activity. The term “therapeutically effective amount” thus includes the amount of the compound or a pharmaceutically acceptable salt or a stereoisomer thereof, that when administered, induces a positive modification in the disease or disorder to be treated, or is sufficient to prevent development or progression of the disease or disorder, or alleviate to some extent, one or more of the symptoms of the disease or disorder being treated in a subject, or which simply kills or inhibits the growth of diseased (e.g., cancer) cells, or reduces the amounts of BCL6 in diseased cells.

The total daily dosage of the compounds of formula (I) and usage thereof may be decided in accordance with standard medical practice, e.g., by the attending physician using sound medical judgment. The specific therapeutically effective dose for any particular subject may depend upon a variety of factors including the disease or disorder being treated and the severity thereof (e.g., its present status); the age, body weight, general health, sex and diet of the subject; the time of administration, route of administration, and rate of excretion of the specific compound employed; the duration of the treatment; drugs used in combination or coincidental with the compound; and like factors well known in the medical arts (see, for example, Goodman and Gilman's, The Pharmacological Basis of Therapeutics, 10th Edition, A. Gilman, J. Hardman and L. Limbird, eds., McGraw-Hill Press, 155-173, 2001).

### METHODS OF USE

In some aspects, the present disclosure is directed to treating a cancer characterized or mediated by aberrant (e.g., elevated levels of BCL6 or otherwise functionally abnormal e.g., deregulated BCL6 levels) BCL6 activity relative to a non-pathological state. The methods entail administering a therapeutically effective amount of a compound of formula (I) or a pharmaceutically acceptable salt or stereoisomer thereof, to a subject in need thereof.

The term “subject” (or “patient”) as used herein includes all members of the animal kingdom prone to or suffering from the indicated disease or disorder. In some embodiments, the subject is a mammal, e.g., a human or a non-human mammal. The methods are also applicable to companion animals such as dogs and cats. A subject “in need of” treatment according to the present disclosure may be “suffering from or suspected of suffering from” a specific disease or disorder may have been positively diagnosed or otherwise presents with a sufficient number of risk factors or a sufficient number or combination of signs or symptoms such that a medical professional could diagnose or suspect that the subject is suffering from the disease or disorder. Thus, subjects suffering from a specific disease or disorder, and subjects suspected of suffering from a specific disease or disorder are not necessarily two distinct groups.

The methods are directed to treating subjects having cancer. Both adult tumors/cancers and pediatric tumors/cancers are included. The cancers may be vascularized, or not yet substantially vascularized, or non-vascularized tumors.

In some embodiments, the methods are directed to treating subjects having a lymphoid malignancy.

In some embodiments, the lymphoid malignancy is peripheral T-cell lymphoma (PTCL), diffuse large B-cell lymphoma (DLBCL), mantle cell lymphoma (MCL), follicular lymphoma (FL), chronic lymphocytic leukemia (CLL), acute lymphoblastic leukemia/lymphoma (ALL), cutaneous T-cell lymphoma, chronic myeloid leukemia, or B-cell non-Hodgkin's lymphoma.

In some embodiments, methods of the present disclosure entail treatment of subjects having cell proliferative diseases or disorders of the hematological system.

Compounds of formula (I) may be administered to a patient, e.g., a cancer patient, as a monotherapy or by way of combination therapy. Therapy may be “front/first-line”, i.e., as an initial treatment in patients who have undergone no prior anti-cancer treatment regimens, either alone or in combination with other treatments; or “second-line”, as a treatment in patients who have undergone a prior anti-cancer treatment regimen, either alone or in combination with other treatments; or as “third-line”, “fourth-line”, etc. treatments, either alone or in combination with other treatments. Therapy may also be given to patients who have had previous treatments which were unsuccessful or partially successful but who became unresponsive or intolerant to the particular treatment. Therapy may also be given as an adjuvant treatment, i.e., to prevent reoccurrence of cancer in patients with no currently detectable disease or after surgical removal of a tumor. Thus, in some embodiments, the compounds may be administered to a patient who has received another therapy, such as chemotherapy, radioimmunotherapy, surgical therapy, immunotherapy, radiation therapy, targeted therapy or any combination thereof.

The methods of the present disclosure may entail administration of a compound of formula (I) or a pharmaceutical composition thereof to the patient in a single dose or in multiple doses (e.g., 1, 2, 3, 4, 5, 6, 7, 8, 10, 15, 20, or more doses). For example, the frequency of administration may range from once a day up to about once every eight weeks. In some embodiments, the frequency of administration ranges from about once a day for 1, 2, 3, 4, 5, or 6 weeks, and in other embodiments entails at least one 28-day cycle which includes daily administration for 3 weeks (21 days) followed by a 7-day “off” period. In other embodiments, the compound may be dosed twice a day (BID) over the course of two and a half days (for a total of 5 doses) or once a day (QD) over the course of two days (for a total of 2 doses). In other embodiments, the compound may be dosed once a day (QD) over the course of 5 days.

### Combination Therapy

The compounds of formula (I) and their pharmaceutically acceptable salts and stereoisomers may be used in combination or concurrently with at least one other active agent, e.g., anti-cancer agent or regimen, in treating diseases and disorders. The terms “in combination” and “concurrently” in this context mean that the agents are co-administered, which includes substantially contemporaneous administration, by way of the same or separate dosage forms, and by the same or different modes of administration, or sequentially, e.g., as part of the same treatment regimen, or by way of successive treatment regimens. Thus, if given sequentially, at the onset of administration of the second compound, the first of the two compounds is in some cases still detectable at effective concentrations at the site of treatment. The sequence and time interval may be determined such that they can act together (e.g., synergistically) to provide an increased benefit than if they were administered otherwise. For example, the therapeutics may be administered at the same time or sequentially in any order at different points in time; however, if not administered at the same time, they may be administered sufficiently close in time so as to provide the desired therapeutic effect, which may be in a synergistic fashion. Thus, the terms are not limited to the administration of the active agents at exactly the same time.

In some embodiments, the treatment regimen may include administration of a compound of formula (I) in combination with one or more additional therapeutics known for use in treating a disease or condition (e.g., cancer). The dosage of the additional therapeutic may be the same or even lower than known or recommended doses. See, Hardman et al., eds., Goodman & Gilman's the Pharmacological Basis of Basis of Therapeutics, 10th ed., McGraw-Hill, New York, 2001; Physician's Desk Reference 60th ed., 2006. For example, anti-cancer agents that may be suitable for use in combination with the compounds are known in the art. See, e.g., U.S. Pat. No. 9,101,622 (Section 5.2 thereof) and U.S. Pat. No. 9,345,705 B2 (Columns 12-18 thereof). Representative examples of additional anti-cancer agents and treatment regimens include radiation therapy, chemotherapeutics (e.g., mitotic inhibitors, angiogenesis inhibitors, anti-hormones, autophagy inhibitors, alkylating agents, intercalating antibiotics, growth factor inhibitors, anti-androgens, signal transduction pathway inhibitors, anti-microtubule agents, platinum coordination complexes, HDAC inhibitors, proteasome inhibitors, and topoisomerase inhibitors), immunomodulators, therapeutic antibodies (e.g., mono-specific and bispecific antibodies) and CAR-T therapy.

In some embodiments, a compound of formula (I) and the additional (e.g., anticancer) therapeutic may be administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. The two or more (e.g., anticancer) therapeutics may be administered within the same patient visit.

When the active components of the combination are not administered in the same pharmaceutical composition, it is understood that they can be administered in any order to a subject in need thereof. For example, a compound of the present disclosure can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the additional therapeutic, to a subject in need thereof. In various aspects, the therapeutics are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one example, the (e.g., anticancer) therapeutics are administered within the same office visit. In another example, the combination anticancer therapeutics may be administered at 1 minute to 24 hours apart.

In some embodiments involving cancer treatment, a compound of formula (I) and the additional anti-cancer agent or therapeutic are cyclically administered. Cycling therapy involves the administration of one anticancer therapeutic for a period of time, followed by the administration of a second anti-cancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

In some embodiments, the compound of the present disclosure may be used in combination with other anti-cancer agents, examples of which include Etoposide (e.g., lymphomas, and non-lymphocytic leukemia), Vincristine (e.g., leukemia), Daunorubicin (e.g., acute myeloid leukemia (AML), acute lymphocytic leukemia (ALL), chronic myelogenous leukemia (CML), and Kaposi's sarcoma), Rituximab (e.g., non-Hodgkin's lymphoma), Alemtuzumab (e.g., chronic lymphocytic leukemia (CLL), cutaneous T-cell lymphoma (CTCL) and T-cell lymphoma), Bortezomib (e.g., multiple myeloma and mantle cell lymphoma), Pegaspargase (e.g., acute lymphoblastic leukemia), Keytruda® (e.g., Hodgkin lymphoma), and dexamethasone (e.g., acute multiple myeloma).

In some embodiments, the additional anti-cancer agent is an enhancer of zeste homolog 2 (EZH2) inhibitor, examples of which include tazemetostat, GSK126, lirametostat (CPI-1205), CPI-0209, PF-06821497, SHR2554, HH2853, valemetostat (DS3201), MAK-683, and FTX-6058.

These and other aspects of the present disclosure will be further appreciated upon consideration of the following Examples, which are intended to illustrate certain particular embodiments of the disclosure but are not intended to limit its scope, as defined by the claims.

### EXAMPLES

### Example 1: Synthesis of 3-(7-((5-chloro-2-((3S,5R)-3,5-dimethylpiperidin-1-yl)pyrimidin-4-yl)amino)-4-methyl-2,3-dioxo-3,4-dihydroquinoxalin-1(2H)-yl)-N-methylpropanamide (1)

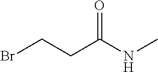

### 3-Bromo-N-methylpropanamide

To a mixture of 3-bromopropanoyl chloride (2 g, 11.67 mmol, 1.18 mL, 1 eq) in DCM (35 mL), MeNH₂ (2M in THF, 11.67 mL, 2 eq) was added dropwise at 0° C. under N₂ atmosphere. The mixture was then allowed to warm to 15° C. and stirred for 12 hours. The mixture was concentrated in vacuo. Then, the resulting residue was diluted with saturated (sat.) NaHCO₃ (50 mL) and extracted with DCM (50 mL*2). The combined organic phase was further washed with sat. NaHCO₃ (50 mL), dried with anhydrous Na₂SO₄, filtered, and concentrated in vacuo to give 3-bromo-N-methylpropanamide (1.2 g, 4.05 mmol, 34.69% yield, 56% purity) as a yellow solid. ¹H NMR (400 MHz, CDCl₃) δ=5.83 (s, 1H), 3.64 (t, J=6.4 Hz, 2H), 2.84 (d, J=4.8 Hz, 3H), 2.76 (t, J=6.4 Hz, 2H).

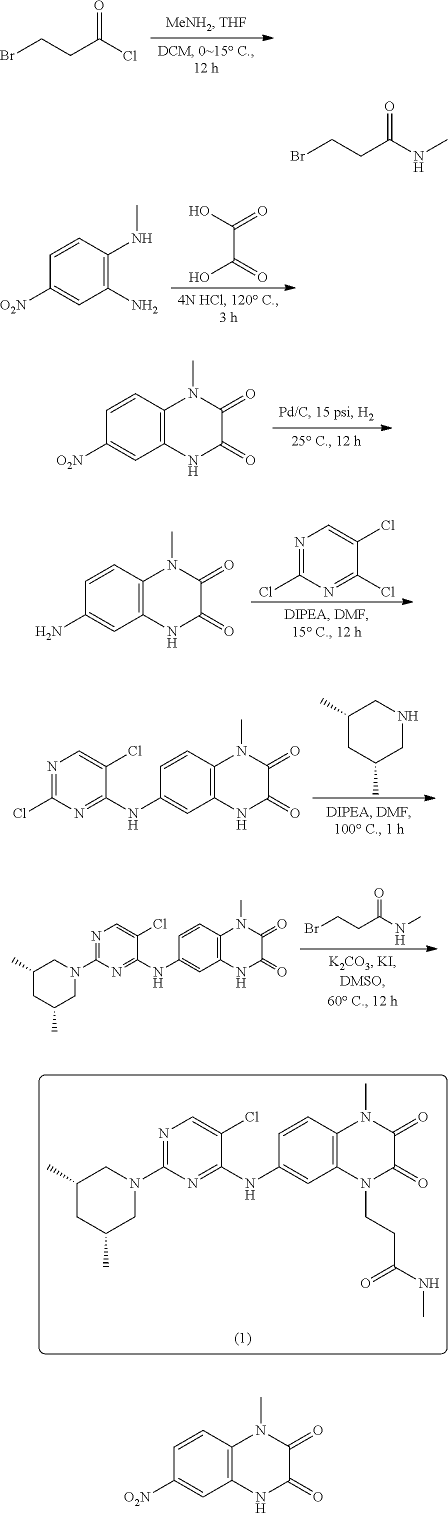

### 1-Methyl-6-nitroquinoxaline-2,3(1H,4H)-dione

A mixture of N1-methyl-4-nitro-benzene-1,2-diamine (3 g, 17.95 mmol, 1 eq) and oxalic acid (3.23 g, 35.89 mmol, 3.17 mL, 2 eq) in aqueous (aq.) 4N HCl (150 mL) was stirred at 120° C. for 3 hours. The mixture was cooled to 15° C., and a precipitate was observed. The mixture was filtered, and the isolated cake was washed with water (50 mL*2) and concentrated in vacuo to give 1-methyl-6-nitroquinoxaline-2,3(1H,4H)-dione (3 g, 13.42 mmol, 74.9% yield, 98.9% purity) as a brown solid. ¹H NMR (400 MHz, DMSO-d₆) δ=12.27 (s, 1H), 8.00 (dd, J=2.8, 9.2 Hz, 1H), 7.96 (d, J=2.4 Hz, 1H), 7.52 (d, J=9.2 Hz, 1H), 3.53 (s, 3H). LCMS: [M+H⁺]=222.1.

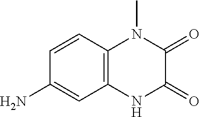

### 6-Amino-1-methylquinoxaline-2,3(1H,4H)-dione

To a solution of 4-methyl-7-nitro-1H-quinoxaline-2,3-dione (1 g, 4.52 mmol, 1 eq) in DMF (50 mL) was added Pd/C (0.2 g, 10% purity) under argon. The suspension was degassed under vacuum and purged several times with hydrogen. The mixture was then stirred under hydrogen (15 psi) at 25° C. for 12 hours. The mixture was filtered through Celite®, and the filtrate was concentrated in vacuo to give 6-amino-1-methylquinoxaline-2,3(1H,4H)-dione (900 mg, crude) as a yellow solid. LCMS: [M+H⁺]=192.1.

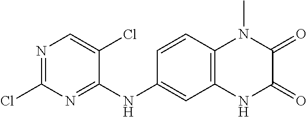 [SMILES: Cn1c(=O)c(=O)[nH]c2cc(Nc3nc(Cl)ncc3Cl)ccc21]

### 6-((2,5-Dichloropyrimidin-4-yl)amino)-1-methylquinoxaline-2,3(1H,4H)-dione

To a mixture of 2,4,5-trichloropyrimidine (863.46 mg, 4.71 mmol, 2 eq) in DMF (5 mL) was added N,N-diisopropylethylamine (DIPEA) (608.4 mg, 4.71 mmol, 819.9 μL, 2 eq). The reaction mixture was stirred at 15° C. for 10 minutes before the dropwise addition of a solution of 7-amino-4-methyl-1H-quinoxaline-2,3-dione (450 mg, 2.35 mmol, 1 eq) in DMF (10 mL). The resulting mixture was stirred at 15° C. for 12 hours. Water (20 mL) was added to the mixture to obtain a precipitate. The resulting mixture was filtered, and the isolated cake was washed with water (30 mL), followed by EtOAc (50 mL). The residual solvent was removed in vacuo to give 6-((2,5-dichloropyrimidin-4-yl)amino)-1-methylquinoxaline-2,3(1H,4H)-dione (300 mg, 789.59 μmol, 33.6% yield, 89.0% purity) as a gray solid. ¹H NMR (400 MHz, DMSO-d₆) δ=8.34 (s, 1H), 7.44-7.31 (m, 3H), 3.54 (s, 3H). LCMS: [M+H⁺]=338.0.

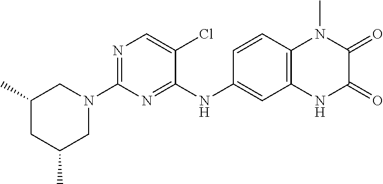 [SMILES: C[C@@H]1C[C@H](C)CN(c2ncc(Cl)c(Nc3ccc4c(c3)[nH]c(=O)c(=O)n4C)n2)C1]

### 6-((5-Chloro-2-((3S,5R)-3,5-dimethylpiperidin-1-yl)pyrimidin-4-yl)amino)-1-methylquinoxaline-2,3(1H,4H)-dione

To a mixture of 7-[(2,5-dichloropyrimidin-4-yl)amino]-4-methyl-1H-quinoxaline-2,3-dione (100 mg, 295.73 μmol, 1 eq) and (3S,5R)-3,5-dimethylpiperidine (40.17 mg, 354.87 μmol, 1.2 eq) in DMF (2 mL) was added DIPEA (76.44 mg, 591.5 μmol, 103.0 μL, 2 eq) under nitrogen atmosphere. The reaction mixture was stirred at 100° C. for 1 hour. The resulting mixture was concentrated in vacuo, and the isolated residue was purified by preparative high-performance liquid chromatography (prep-HPLC) (column: Waters™ Xbridge BEH C18 100*30 mm*10 μm; mobile phase: [water (10 mM NH₄HCO₃)-ACN]; B %: 40%-70%, 10 min) to give 6-((5-chloro-2-((3S,5R)-3,5-dimethylpiperidin-1-yl)pyrimidin-4-yl)amino)-1-methylquinoxaline-2,3(1H,4H)-dione (99% purity, 26.2 mg) as a white solid. ¹H NMR (400 MHz, DMSO-d₆) δ=12.09 (s, 1H), 8.87 (s, 1H), 8.02 (s, 1H), 7.49-7.43 (m, 2H), 7.34-7.28 (m, 1H), 4.48 (d, J=10.8 Hz, 2H), 3.52 (s, 3H), 2.25 (t, J=12.4 Hz, 2H), 1.76 (d, J=13.2 Hz, 1H), 1.57-1.43 (m, 2H), 0.85 (d, J=6.4 Hz, 6H), 0.77 (q, J=12.0 Hz, 1H). LCMS: [M+H⁺]=415.1.

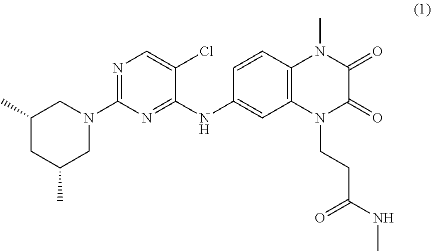

To a mixture of 7-[[5-chloro-2-[(3S,5R)-3,5-dimethyl-1-piperidyl]pyrimidin-4-yl]amino]-4-methyl-1H-quinoxaline-2,3-dione (100 mg, 241.03 μmol, 1 eq) in DMSO (10 mL), 3-bromo-N-methyl-propanamide (44.02 mg, 265.13 μmol, 1.1 eq), K₂CO₃ (39.98 mg, 289.23 μmol, 1.2 eq) and KI (20.01 mg, 120.51 μmol, 0.5 eq) were added. The mixture was stirred at 60° C. for 12 hours, concentrated in vacuo, and filtered to remove solids. The isolated mixture was purified by prep-HPLC (column: Waters™ Xbridge BEH C18 100*25 mm*5 μm; mobile phase: [water(10 mM NH₄HCO₃)-ACN]; B %: 35%-65%, 10 min) to give 3-(7-((5-chloro-2-((3S,5R)-3,5-dimethylpiperidin-1-yl)pyrimidin-4-yl)amino)-4-methyl-2,3-dioxo-3,4-dihydroquinoxalin-1(2H)-yl)-N-methylpropanamide (51.5 mg, 42.7% yield) as a white solid. ¹H NMR (400 MHz, DMSO-d₆) δ=8.87 (s, 1H), 8.05 (s, 1H), 7.94 (dd, J=2.0, 3.6 Hz, 1H), 7.79-7.71 (m, 1H), 7.65 (s, 1H), 7.38 (d, J=8.8 Hz, 1H), 4.53-4.49 (m, 2H), 4.31-4.21 (m, 2H), 3.53 (s, 3H), 2.55 (d, J=4 Hz, 3H), 2.48-2.43 (m, 2H), 2.27 (t, J=12.0 Hz, 2H), 1.81-1.72 (m, 1H), 1.59-1.44 (m, 2H), 0.86 (d, J=6.4 Hz, 6H), 0.77 (q, J=12.0 Hz, 1H). LCMS: [M+H⁺]=500.1.

### Example 2: Synthesis of 2-(7-((5-chloro-2-((3R,5S)-4,4-difluoro-3,5-dimethylpiperidin-1-yl) pyrimidin-4-yl)amino)-4-methyl-2,3-dioxo-3,4-dihydroquinoxalin-1(2H)-yl)-N-methylacetamide (56)

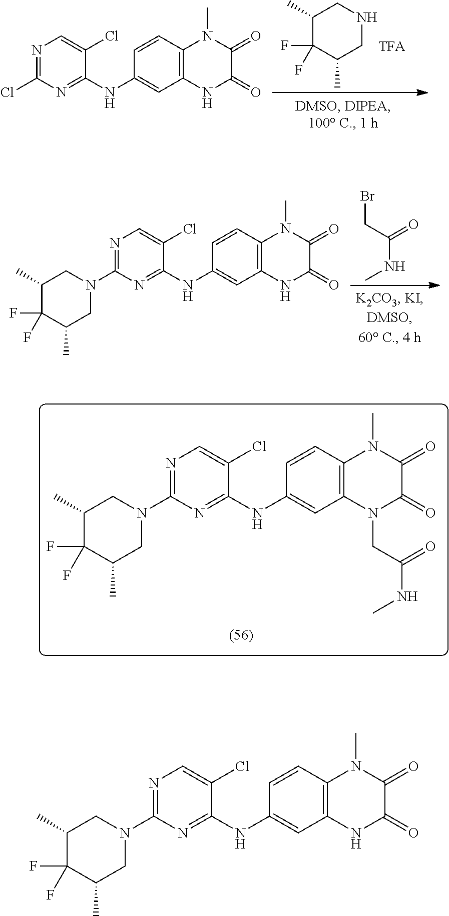

### 6-((5-Chloro-2-((3R,5S)-4,4-difluoro-3,5-dimethylpiperidin-1-yl)pyrimidin-4-yl)amino)-1-methylquinoxaline-2,3(1H,4H)-dione

A mixture of 7-[(2,5-dichloropyrimidin-4-yl)amino]-4-methyl-1H-quinoxaline-2,3-dione (2 g, 5.91 mmol, 1 eq), (3S,5R)-4,4-difluoro-3,5-dimethyl-piperidine (2.02 g, 7.69 mmol, 1.3 eq, TFA), and DIPEA (1.53 g, 11.83 mmol, 2.06 mL, 2 eq) in DMSO (20 mL) was stirred at 100° C. for 3 hours under nitrogen atmosphere. Water (20 mL) was added to the mixture to obtain a precipitate. The resulting mixture was filtered, and the isolated cake was washed with water (50 mL), followed by EtOAc (50 mL). The isolated residue was dried under reduced pressure to get 7-[[5-chloro-2-[(3S,5R)-4,4-difluoro-3,5-dimethyl-1-piperidyl]pyrimidin-4-yl]amino]-4-methyl-1H-quinoxaline-2,3-dione (2.2 g, 4.60 mmol, 77.8% yield, 94.1% purity) as a white solid. ¹H NMR (400 MHz, DMSO-d₆) δ=12.10 (br s, 1H), 8.98 (s, 1H), 8.07 (s, 1H), 7.46 (d, J=2.0 Hz, 1H), 7.43-7.37 (m, 1H), 7.34-7.28 (m, 1H), 4.49 (br d, J=11.6 Hz, 2H), 3.51 (s, 3H), 2.64 (br t, J=12.8 Hz, 2H), 2.12-1.92 (m, 2H), 0.96 (d, J=6.4 Hz, 6H).

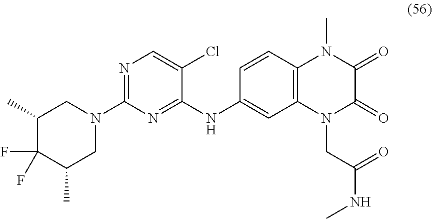 [SMILES: CNC(=O)Cn1c(=O)c(=O)n(C)c2ccc(Nc3nc(N4C[C@@H](C)C(F)(F)[C@@H](C)C4)ncc3Cl)cc21]

To a solution of 7-[[5-chloro-2-[(3S,5R)-4,4-difluoro-3,5-dimethyl-1-piperidyl]pyrimidin-4-yl]amino]-4-methyl-1H-quinoxaline-2,3-dione (100 mg, 221.79 μmol, 1 eq) and 2-bromo-N-methyl-acetamide (50.57 mg, 332.69 μmol, 1.5 eq) in DMSO (1.5 mL), K₂CO₃ (61.31 mg, 443.59 μmol, 2 eq) and KI (18.41 mg, 110.90 μmol, 0.5 eq) were added, and the mixture was stirred at 60° C. for 4 hours. Water was added to the reaction mixture to obtain a precipitate. The resulting mixture was filtered, and the isolated cake was washed with water (15 mL*2) and then EtOH (15 mL*2). Residual solvent was removed under reduced pressure, and the resulting residue was triturated with DMSO (2 ml) at 15° C. for 10 minutes before filtering the mixture. The isolated cake was concentrated under reduced pressure to get 2-[7-[[5-chloro-2-[(3S,5R)-4,4-difluoro-3,5-dimethyl-1-piperidyl]pyrimidin-4-yl]amino]-4-methyl-2,3-dioxo-quinoxalin-1-yl]-N-methyl acetamide (37 mg, 96.7% purity) as a white solid. ¹H NMR (400 MHz, DMSO-d₆) δ=8.98 (s, 1H), 8.10 (s, 1H), 8.03 (br d, J=4.4 Hz, 1H), 7.75 (br d, J=8.8 Hz, 1H), 7.41 (br d, J=8.8 Hz, 1H), 7.38 (br s, 1H), 4.63 (br s, 2H), 4.51 (br d, J=10.4 Hz, 2H), 3.57 (s, 3H), 2.68 (br t, 1=12.8 Hz, 2H), 2.57 (br d, J=4 Hz, 3H), 2.13-1.95 (m, 2H), 0.98 (br d, J=6.8 Hz, 6H). LCMS: [M+H⁺]=522.18.

### Example 3: Degradation Activity in SU-DHL-4 Cells

### HiBiT Protocol

DC₅₀ (concentration to reach 50% degradation) values were determined from a cellular degradation assay (HiBiT, Promega™) in Su-DHL-4 cells (Table 1). Endogenous BCL6 was tagged with the 11-amino acid SmBiT through CRISPR/Cas9 gene editing and single cell clone selection. After 24 hours of compound treatment, cells were lysed and incubated with LgBiT protein to reconstitute intact nanoluciferase. Substrate was then added and relative luciferase units were measured. Degradation levels for each treatment were taken as a percentage compared to the control, 100% DMSO (Prism).

TABLE 1
DC₅₀ in SU-DHL-4 cells and MS data

| Compound | MS [M + H⁺] | HiBiT SU-DHL-4 DC₅₀ (μM) |
|---|---|---|
| 1 | 500.1 | 0.025 |
| 8 | 591.2 | >10 |
| 9 | 605.3 | 0.22 |
| 11 | 605.2 | 3.1 |
| 12 | 619.3 | 1.8 |
| 18 | 593.3 | 0.45 |
| 19 | 607.3 | 0.5 |
| 28 | 578.1 | 0.032 |
| 29 | 592.2 | 0.071 |
| 31 | 592.1 | 0.16 |
| 32 | 606.2 | 0.15 |
| 56 | 522.2 | 0.014 |
| 57 | 579.2 | 0.34 |
| 58 | 577.2 | 0.87 |
| 59 | 591.2 | 0.97 |
| 60 | 605.2 | 1.12 |
| 68 | 564.2 | 0.1 |
| 73 | 594.2 | 0.13 |
| 75-1 | 562.1 | 0.29 |
| 75-2 | 562.1 | 0.1 |
| 80 | 565.2 | >10 |
| 81 | 565.2 | 0.25 |
| 82 | 537.1 | 0.084 |
| 83 | 537.1 | 0.92 |
| 84 | 538.1 | N/A |
| 85 | 538.1 | 0.094 |
| 86 | 523.2 | 0.12 |
| 87 | 523.2 | 0.23 |
| 106 | 487.2 | 0.041 |
| 107 | 487.2 | 0.047 |
| 108 | 517.1 | 0.043 |
| 109 | 517.1 | 0.004 |
| 110 | 517.2 | 0.018 |
| 111 | 531.2 | 0.034 |
| 112 | 533.2 | 0.025 |
| 117 | 532.2 | 0.13 |
| 118 | 532.2 | 0.12 |
| 119 | 541.1 | 0.013 |
| 120 | 590.1 | 1.9 |
| 121 | 590.1 | 0.071 |
| 122 | 505.1 | 0.019 |
| 123 | 505.2 | 0.023 |

The data for the HiBiT assay in SU-DHL-4 (B cell lymphoma) cells set forth in Table 1 show that the compound of Formula I demonstrated DC₅₀ values of 0.1 μM or less.

All patent publications and non-patent publications are indicative of the level of skill of those skilled in the art to which this disclosure pertains. All these publications are herein incorporated by reference to the same extent as if each individual publication were specifically and individually indicated as being incorporated by reference.

Although the disclosure herein has been described with reference to particular embodiments, it is to be understood that these embodiments are merely illustrative of the principles and applications of the present disclosure. It is therefore to be understood that numerous modifications may be made to the illustrative embodiments and that other arrangements may be devised without departing from the spirit and scope of the present invention as defined by the appended claims.

What is claimed is:

1. A compound having a structure represented by formula I: 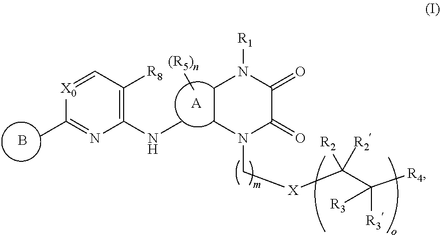
or a pharmaceutically acceptable salt or stereoisomer thereof,
wherein: 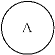
is phenyl or pyridyl; 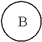
is 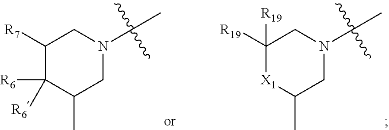
X is a bond, (C₃-C₆) carbocyclyl, (C₃-C₆) carbocyclyl(C═O), or SO₂;
X₀ is N or CCN;
X₁ is CH₂, S, CHF, CHCl, CHOH, or CF₂;
R₁ is (C₁-C₆) alkyl, (C₁-C₆) hydroxyalkyl, (C₁-C₆) aminoalkyl, (C₃-C₆) carbocyclyl, 4- to 6-membered heterocyclyl, (C₁-C₆) alkyl-(C₃-C₆) carbocyclyl, or (C₁-C₆) alkyl-4- to 6-membered heterocyclyl, wherein said alkyl, hydroxyalkyl, aminoalkyl, carbocyclyl, or heterocyclyl is further optionally substituted by one or more, identical or different R₁ₐ groups, wherein R₁ₐ is deuterium, (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl;
R₂ and R_{2′} are each independently H, (C₁-C₃) alkyl, (C₁-C₃) hydroxyalkyl, or (C₁-C₃) aminoalkyl,
or R₂ and R_{2′}, together with the same carbon atom to which they are attached, form (C₃-C₆) carbocyclyl or 4- to 6-membered heterocyclyl, wherein said alkyl, hydroxyalkyl, aminoalkyl, carbocyclyl, or heterocyclyl is optionally substituted by one or more, identical or different R₂ₐ groups, wherein R₂ₐ is (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl;
R₃ and R_{3′} are each independently H, (C₁-C₃) alkyl, (C₁-C₃) hydroxyalkyl, or (C₁-C₃) aminoalkyl, or
R₃ and R_{3′}, together with the same carbon atom to which they are attached, form C═O, (C₃-C₆) carbocyclyl, 4- to 6-membered heterocyclyl, wherein said carbocyclyl or heterocyclyl is optionally substituted by one or more, identical or different R₃ₐ groups, wherein each R₃ₐ is independently (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl, or wherein two R₃ₐ groups, together with the same carbon atom to which they are attached, form C═O,
or wherein R₂ or R_{2′} and R₃ or R_{3′}, together with the same carbon atom to which they are attached, form (C₃-C₆) carbocyclyl or 4- to 6-membered heterocyclyl, wherein said carbocyclyl or heterocyclyl is optionally substituted by one or more, identical or different R₂ₐ groups;
R₄ is H, OH, NH—(C₁-C₆) alkyl, NH—(C₁-C₆) hydroxyalkyl, NH—(C₁-C₆) aminoalkyl, NH—(C₃-C₆) carbocyclyl, NH-4- to 6-membered heterocyclyl, NH—(C₁-C₆) alkyl-(C₃-C₆) carbocyclyl, NH—(C₁-C₆) alkyl-4- to 6-membered heterocyclyl, wherein said alkyl, hydroxyalkyl, aminoalkyl, carbocyclyl, or heterocyclyl is further optionally substituted by one or more, identical or different R₄ₐ groups, wherein each R₄ₐ is independently (C₁-C₆) alkyl, (C₁-C₆) alkoxy, (C₁-C₆) alkyl-(C₁-C₃) alkoxy, halogen, amino, hydroxyl, (C₁-C₆) haloalkyl, NH—(C₁-C₆) alkyl, N((C₁-C₆)alkyl)₂, (C₃-C₆) carbocyclyl, or 4- to 6-membered heterocyclyl;
R₅ is halogen or (C₁-C₃) alkoxy;
R₆ and R_{6′} are each independently H or halogen;
R₇ is H, halogen, (C₁-C₄) alkyl, (C₁-C₄) alkoxy, (C₁-C₄) haloalkyl, (C₁-C₄) haloalkoxy, CN, N₀₂, OH, (C₂-C₄) alkenyl, (C₂-C₄) alkynyl, or —R₇ₐ-R_{7b}, wherein R₇ₐ is absent or O, N(R₇ₐ₁)(CR₇ₐ₁R₇ₐ₂)_{q}, S, SO, SO₂, C(O), C(O)O, OC(O), C(O)N(R₇ₐ₁), N(R₇ₐ₁)C(O), N(R₇ₐ₁)C(O)N(R₇ₐ₂), N(R₇ₐ₁)C(O)O, OC(O)N(R₇ₐ₁), S(O)₂N(R₇ₐ₁), N(R₇ₐ₁)SO₂, wherein R₇ₐ₁ and R₇ₐ₂ are each independently H or (C₁-C₄) alkyl, and q is 0, 1, 2, and R_{7b} is H, (C₁-C₆) alkyl, (C₆-C₁₀) aryl, (C₃-C₆) cycloalkyl, (C₂-C₄) alkenyl, (C₁-C₄) alkynyl, (C₃-C₆) cycloalkenyl, wherein said alkyl, aryl, cycloalkyl, alkenyl, alkynyl, or cycloalkenyl is optionally further substituted by one or more substituent groups independently selected from oxo, (C₁-C₄) alkyl, (C₁-C₄) cycloalkyl, halogen, (C₁-C₄) haloalkyl, (C₁-C₄) haloalkoxy, (C₁-C₄) hydoxyalkyl, amino, CN, OH, amido, carboxy, carbamoyl, sulphamoyl, mercapto, C(O)NR_{7b1}R_{7b2}, R_{7b1}R_{7b2}, or OR_{7b1}, wherein R_{7b1} and R_{7b2} each is independently selected from hydrogen, (C₁-C₄) alkyl, (C₃-C₆) cycloalkyl, wherein said oxo, alkyl, cycloalkyl, haloalkyl, haloalkoxy, hydoxyalkyl, amino, CN, OH, amido, carboxy, carbamoyl, sulphamoyl, or mercapto is optionally further substituted by R_{7b3}—R_{7b4}, wherein R_{7b3} is absent or (C₁-C₅) alkylene optionally substituted by one or more substituents selected from (C₁-C₂) alkyl or oxo, and R_{7b4} is (C₆-C₁₀) aryl, 5- to 12-membered heteroaryl, 4- to 12-membered heterocyclyl, (C₃-C₆) carbocyclyl, halogen, (C₁-C₄) haloalkyl, (C₁-C₄) haloalkoxy, CN, OH, (C₁-C₄) alkoxy, C(O)R_{7b4a}, COOR_{7b4b}, C(O)NR_{7b4a}R_{7b4b}, or NR_{7b4a}R_{7b4b}, wherein: R_{7b4a} and R_{7b4b} are each independently selected from H or (C₁-C₄) alkyl; said aryl, heteroaryl, heterocyclyl, or carbocyclyl is optionally further substituted by one or more substituent groups independently selected from (C₁-C₄)alkyl, halogen, (C₁-C₄) haloalkyl, amino, CN or OH; and said heterocyclyl or heteroaryl contains at least one nitrogen atom and is linked via nitrogen, wherein said heterocyclyl group is optionally and independently substituted by one or more, identical or different groups independently selected from R₉, and said heteroaryl group is optionally and independently substituted by one or more, identical or different group independently selected from R₁₀;
R₈ is halogen or CN;
R₉ is ═O, CN, C≡CH, OH, COOH, halogen, (C₁-C₆) alkyl, (C₁-C₆) alkoxy, (C₁-C₆) haloalkyl, 5 or 6 membered heteroaryl, phenyl, N(R₁₁R₁₂), C(O)—R₁₃, C(O)N(R₁₄R₁₅), or 5- to 8-membered heterocyclyl, wherein said (C₁-C₆) alkyl is optionally substituted with COOH, OH, COO—(C₁-C₆) alkyl, —CON((C₁-C₆) alkyl)₂, (C₁-C₆) alkoxy, N((C₁-C₃) alkyl)₂, phenyl, or 5- or 6-membered heterocyclyl, and said heteroaryl, phenyl, or heterocyclyl is optionally substituted with one group selected from (C₁-C₆) alkyl;
R₁₀ is COOH, (C₁-C₆) alkyl, C(O)—R₁₆, or C(O)N(R₁₇)(R₁₈);
R₁₁ is H or (C₁-C₄) alkyl;
R₁₂ is H, (C₁-C₄) alkyl, (C₁-C₄) haloalkyl, 6-membered heterocyclyl, or 6-membered heteroaryl;
R₁₃ is (C₁-C₃) alkyl-N((C₁-C₃) alkyl)₂ or 5- or 6-membered heterocyclyl, wherein said heterocyclyl is optionally substituted with (C₁-C₃) alkyl;
R₁₄ is hydrogen or (C₁-C₃) alkyl;
R₁₅ is (C₁-C₆) alkyl optionally substituted with NH₂, (C₁-C₆) alkoxy, O—(C₁-C₆) alkyl-NH₂, or O—(C₁-C₆) alkyl-O—(C₁-C₆) alkyl-NH₂, or R₁₅ is a 6-membered heterocyclyl optionally substituted with (C₁-C₃) alkyl;
R₁₆ is a 6-membered heterocyclyl optionally substituted with (C₁-C₃) alkyl;
R₁₇ and R₁₈ are each independently H or (C₁-C₃) alkyl;
Rig is H, ═O, —CN, —C≡CH, —OH, —SH, —NH₂, —COOH, halo, (C₁-C₆)alkyl, —O—(C₁-C₆)alkyl, (C₁-C₆)haloalkyl, amido, carboxy, carbamoyl, sulfamoyl, phenyl, 5- to 8-membered heterocyclyl, —NR₂₀R₂₁, —C(O)R₂₂, —C(O)NR₂₃R₂₄, or L₁Y₁, wherein said alkyl, phenyl, or heterocyclyl is optionally substituted with one or more groups selected from halo, —COOH, —OH, —NH₂, (C₁-C₆)alkyl, —C(O)O—(C₁-C₆)alkyl, —C(O)N(C₁-C₆ alkyl)₂, —O—(C₁-C₆)alkyl, —N(C₁-C₃ alkyl)₂, phenyl, and 4- to 6-membered heterocyclyl, optionally substituted with one or more groups selected from halo and (C₁-C₆)alkyl,
L₁ is absent, (C₁-C₆)alkylene or (C₃-C₇)carbocyclyl; wherein said alkylene or carbocyclyl is further optionally substituted by one or more, identical or different R₂₅ groups, or L₁ is (C₂-C₄)alkylene which is bound to R₂₆ to form a 4- to 6-membered heterocyclyl group; and
Y₁ is —CN, —OH, halo, (C₁-C₄)alkoxy, (C₁-C₄)haloalkyl, (C₁-C₄)haloalkoxy, 4- to 7-membered heterocyclyl, (C₃-C₆)carbocyclyl, —NR₂₆R₂₇, —C(O)R₂₂, —C(O)NR₂₃R₂₄, wherein said alkyl, carbocyclyl, or heterocyclyl is further optionally substituted by one or more, identical or different groups selected from (C₁-C₄)alkyl, halo, (C₁-C₄)haloalkyl, —CN, —OH, and —NH₂;
R₁₉′ is absent, H, —CN, —C≡CH, —OH, —SH, —NH₂, —COOH, halo, (C₁-C₆)alkyl, —O—(C₁-C₆)alkyl, (C₁-C₆)haloalkyl, amido, carboxy, carbamoyl, sulfamoyl, phenyl, 5- to 8-membered heterocyclyl, —NR₂₀R₂₁, —C(O)R₂₂, or —C(O)NR₂₃R₂₄; wherein said alkyl, phenyl, or heterocyclyl is further optionally substituted by one or more, identical or different R₂₅ groups, or
R₁₉′ and L₁ together with the same carbon atom to which they are attached form a spiro (C₃-C₇)carbocyclyl group or a 4- to 7-membered heterocyclyl group; wherein said carbocyclyl or heterocyclyl is further optionally substituted by one or more, identical or different R₂₅ groups;
R₂₀ is hydrogen, (C₁-C₄)alkyl, or (C₃-C₆)cycloalkyl;
R₂₁ is hydrogen, (C₁-C₄)alkyl, (C₁-C₄)haloalkyl, (C₃-C₆)cycloalkyl, or 6-membered heterocyclyl;
R₂₂ is —(C₁-C₃)alkyl-N(C₁-C₃ alkyl)₂, (C₃-C₆)cycloalkyl, or 5- to 6-membered heterocyclyl, wherein said heterocyclyl is optionally substituted with (C₁-C₃)alkyl;
R₂₃ is hydrogen, (C₁-C₃)alkyl, or (C₃-C₆)cycloalkyl;
R₂₄ is (C₃-C₆)cycloalkyl or (C₁-C₆)alkyl optionally substituted with —NH₂, —O—(C₁-C₆)alkyl, —O—(C₁-C₆)alkyl-NH₂, or —O—(C₁-C₆)alkyl-O—(C₁-C₆)alkyl-NH₂;
each R₂₅ is independently oxo, alkyl, alkenyl, alkynyl, halo, haloalkyl, carbocyclyl, heterocyclyl, hydroxy, alkoxy, cycloalkoxy, heterocycloalkoxy, haloalkoxy, aryloxy, heteroaryloxy, aralkyloxy, alkyenyloxy, alkynyloxy, amino, alkylamino, cycloalkylamino, heterocycloalkylamino, arylamino, heteroarylamino, aralkylamino, N-alkyl-N-arylamino, N-alkyl-N-heteroarylamino, N-alkyl-N-aralkylamino, hydroxyalkyl, aminoalkyl, alkylthio, haloalkylthio, alkylsulfonyl, haloalkylsulfonyl, cycloalkylsulfonyl, heterocycloalkylsulfonyl, arylsulfonyl, heteroarylsulfonyl, aminosulfonyl, alkylaminosulfonyl, cycloalkylaminosulfonyl, heterocycloalkylaminosulfonyl, arylaminosulfonyl, heteroarylaminosulfonyl, N-alkyl-N-arylaminosulfonyl, N-alkyl-N-heteroarylaminosulfonyl, formyl, alkylcarbonyl, haloalkylcarbonyl, alkenylcarbonyl, alkynylcarbonyl, carboxy, alkoxycarbonyl, alkylcarbonyloxy, amino, alkylsulfonylamino, haloalkylsulfonylamino, cycloalkylsulfonylamino, heterocycloalkylsulfonylamino, arylsulfonylamino, heteroarylsulfonylamino, aralkylsulfonylamino, alkylcarbonylamino, haloalkylcarbonylamino, cycloalkylcarbonylamino, heterocycloalkylcarbonylamino, arylcarbonylamino, heteroarylcarbonylamino, aralkylsulfonylamino, aminocarbonyl, alkylaminocarbonyl, cycloalkylaminocarbonyl, heterocycloalkylaminocarbonyl, arylaminocarbonyl, heteroarylaminocarbonyl, N-alkyl-N-arylaminocarbonyl, N-alkyl-N-heteroarylaminocarbonyl, cyano, nitro, azido, or phosphinyl;
R₂₆ and R₂₇ are each independently hydrogen, (C₁-C₆)alkyl, (C₃-C₇)carbocyclyl, 4- to 7-membered heterocyclyl, (C₆-C₁₀)aryl, or monocyclic or bicyclic 5- to 10-membered heteroaryl; wherein said alkyl, carbocyclyl, heterocyclyl, aryl or heteroaryl is further optionally substituted by one or more, identical or different R₂₅ groups, or
R₂₆ and R₂₇ together with the nitrogen atom to which they are attached form a 3- to 7-membered heterocyclyl, wherein said heterocyclyl is further optionally substituted by one or more, identical or different R₂₅ groups;
m is 0, 1, or 2;
n is 0, 1, 2, or 3; and
is 0 or 1.

2. The compound of claim 1, wherein 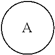
is phenyl and 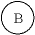
is 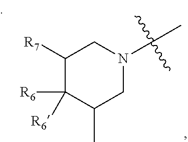
and the compound is represented by structure I-1a: 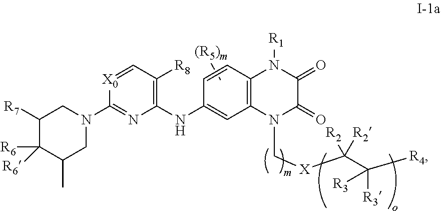
or a pharmaceutically acceptable salt or stereoisomer thereof.

3. The compound of claim 1, wherein 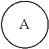
is pyridyl and 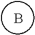
is 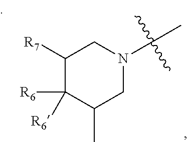
and the compound is represented by any one of structures I-2a to I-4a: 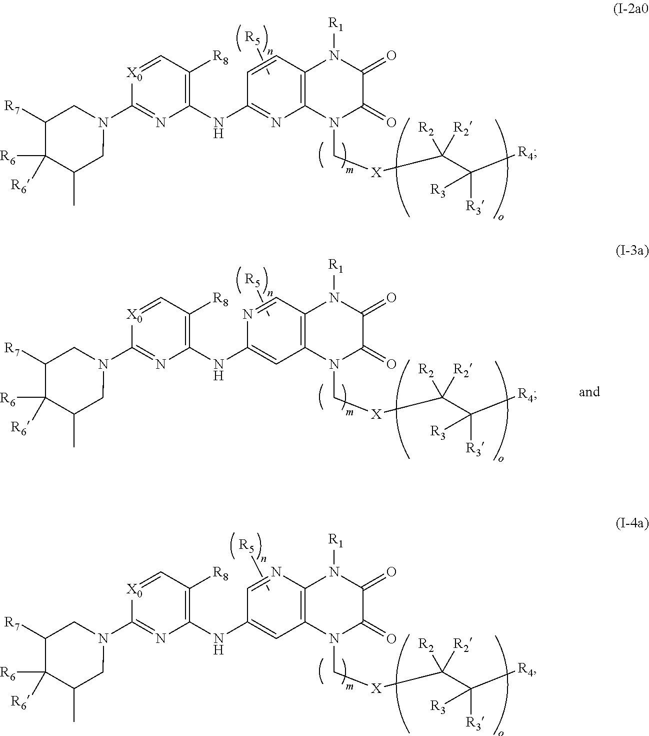
or a pharmaceutically acceptable salt or stereoisomer thereof.

4. The compound of claim 1, wherein R₇ is optionally substituted (C₁-C₄) alkyl, amino, or OH.

5. The compound of claim 4, wherein R₇ is methyl or

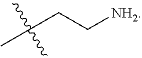 [SMILES: *CCN]

6. (canceled)

7. The compound of claim 1, wherein 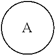
is phenyl and 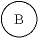
is 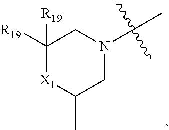
and the compound is represented by structure I-1b: 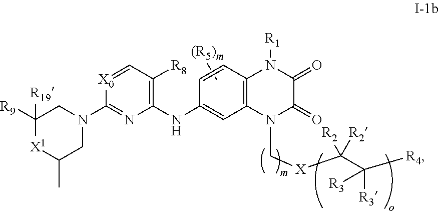
or a pharmaceutically acceptable salt or stereoisomer thereof.

8. The compound of claim 1, wherein 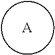
is pyridyl and 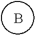
is 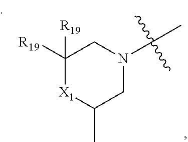
and the compound is represented by any one of structures I-2b to I-4b: 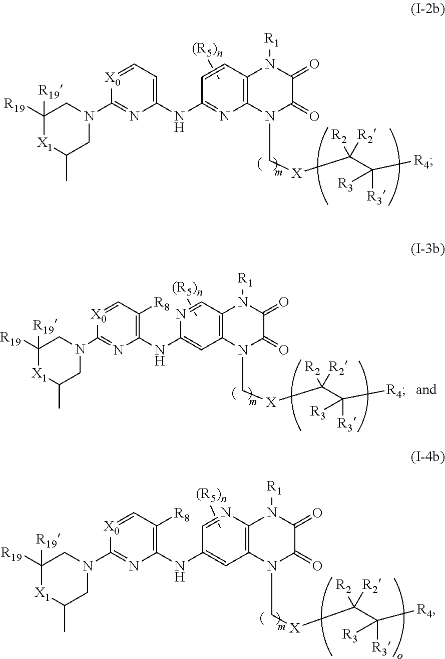
or a pharmaceutically acceptable salt or stereoisomer thereof.

9. The compound of claim 7, wherein X₁ is CH₂, CHF, CHOH, or CF₂.

10. The compound of claim 7, wherein R₁₉ and R₁₉′ are each independently H, OH, methyl, amino,

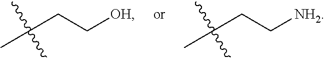

11. The compound of claim 1, wherein R₈ is halogen.

12. The compound of claim 11, wherein R₈ is Cl or F.

13. The compound of claim 1, wherein R₁ is optionally substituted (C₁-C₆) alkyl, optionally substituted (C₁-C₆) hydroxyalkyl, or optionally substituted (C₁-C₆) aminoalkyl.

14. The compound of claim 13, wherein R₁ is methyl,

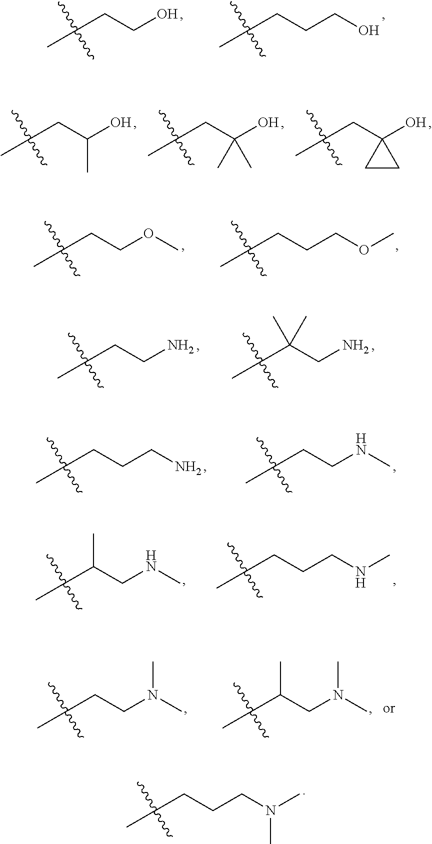

15.-18. (canceled)

19. The compound of claim 1, wherein R₁ is optionally substituted 4- to 6-membered heterocyclyl or optionally substituted (C₁-C₆) alkyl-4- to 6-membered heterocyclyl.

20. The compound of claim 19, wherein R₁ is

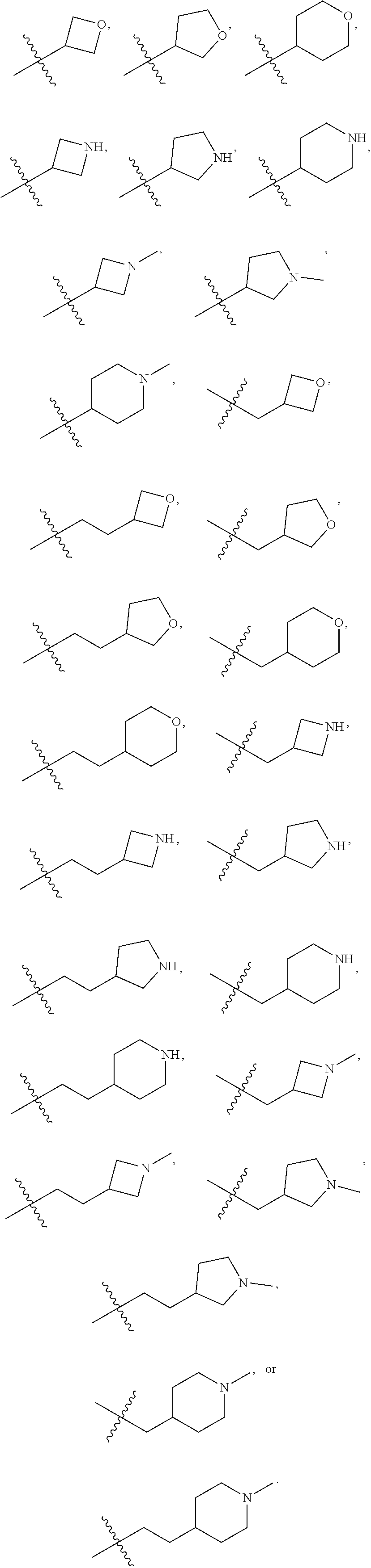

21.-22. (canceled)

23. The compound of claim 1, wherein X is a bond or (C₃-C₆) carbocyclyl, m is 0 or 1, and o is 0 or 1.

24. The compound of claim 1, wherein 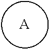
is phenyl and 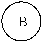
is 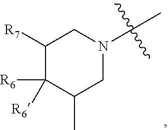
and the compound is represented by structure I-1a1 or I-1b1: 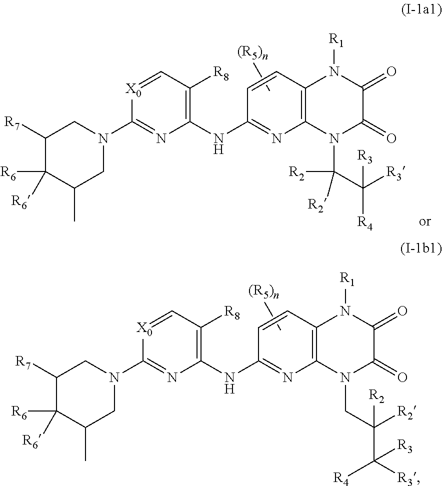
or a pharmaceutically acceptable salt or stereoisomer thereof, or
wherein 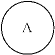
is phenyl, 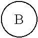
is 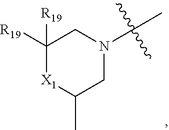
X is a bond, m is 0 or 1, and o is 1, and the compound is represented by structure I-1a2 or I-1b2: 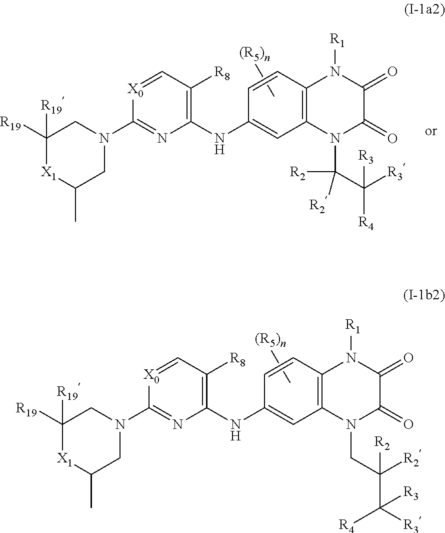
or a pharmaceutically acceptable salt or stereoisomer thereof.

25. (canceled)

26. The compound of claim 1, wherein 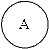
pyridyl and 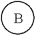
is 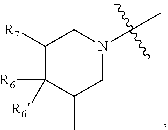
and the compound is represented by any one of structures I-2a1 to I-4a1 and I-2b1 to I-4b1: 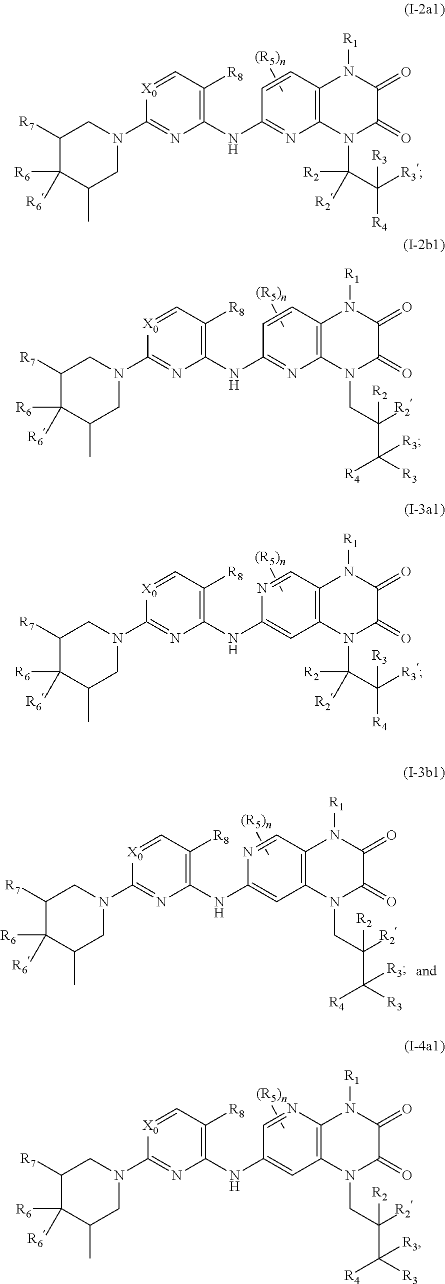
or a pharmaceutically acceptable salt or stereoisomer thereof.

27. The compound of claim 1, wherein 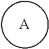
is pyridyl, 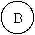
is 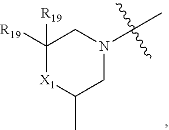
X is a bond, m is 0 or 1, and o is 1, and the compound is represented by any one of structures I-2a2 to I-4a2 and I-2b2 to I-4b2: 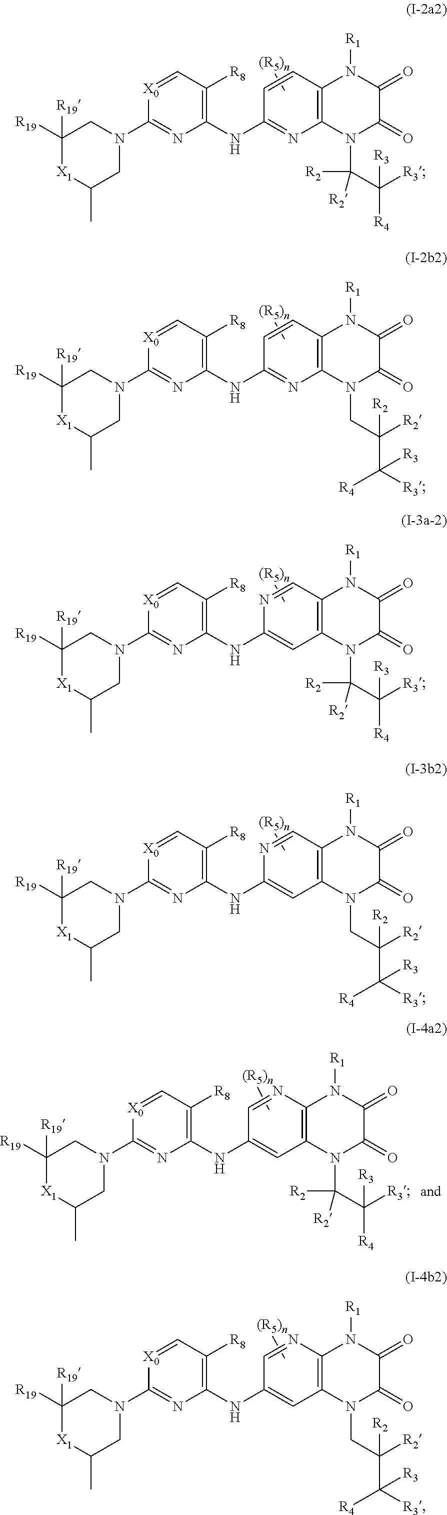
or a pharmaceutically acceptable salt or stereoisomer thereof.

28. The compound of claim 24, wherein R₂ and R_{2′} are each independently H or methyl.

29. The compound of claim 24, wherein both R₂ and R_{2′} are H.

30. The compound of claim 24, wherein R₂ and R_{2′}, together with the same carbon atom to which they are attached, form cyclopropyl.

31. The compound of claim 23, wherein R₃ and R_{3′}, together with the same carbon atom to which they are attached, form C═O.

32. The compound of claim 23, wherein R₃ and R_{3′}, together with the same carbon atom to which they are attached, form optionally substituted 4- to 6-membered heterocyclyl.

33. The compound of claim 32, wherein the optionally substituted 4-membered heterocyclyl is oxetane or N-methylazetidine.

34. The compound of claim 32, wherein the optionally substituted 4- to 6-membered heterocyclyl is carbamate.

35. The compound of claim 1, wherein 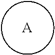
is phenyl, 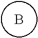
is 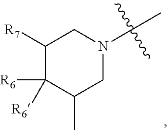
X is C₄ carbocyclyl(C═O), and m and o are 0, and the compound is represented by structure I-1c1: 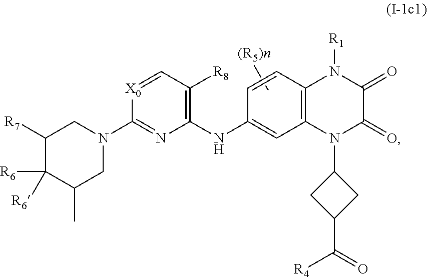
or a pharmaceutically acceptable salt or stereoisomer thereof, or
wherein 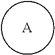
is phenyl 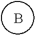
is 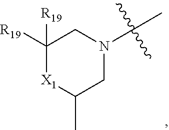
X is C₄ carbocyclyl(C═O), and m and o are 0, and the compound is represented by structure I-1c2: 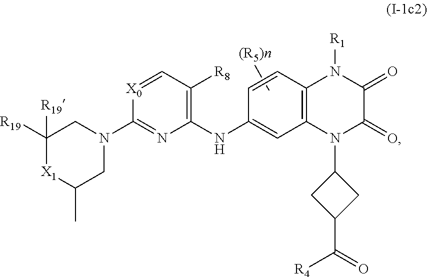
or a pharmaceutically acceptable salt or stereoisomer thereof.

36. (canceled)

37. The compound of claim 1, wherein 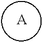
pyridyl, 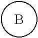
is 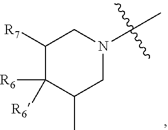
X is C₄ carbocyclyl(C═O), and m and o are 0, and the compound is represented by any one of structures I-2c1 to I-4c1: 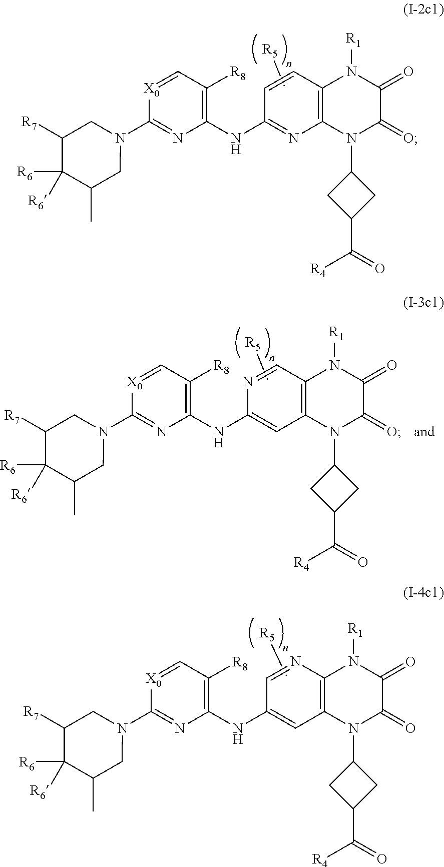
or a pharmaceutically acceptable salt or stereoisomer thereof.

38. The compound of claim 1, wherein 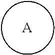
is pyridyl, 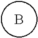
is 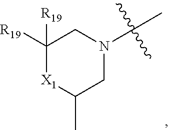
X is C₄ carbocyclyl(C═O), and m and o are 0, and the compound is represented by any one of structures I-2c2 to I-4c2: 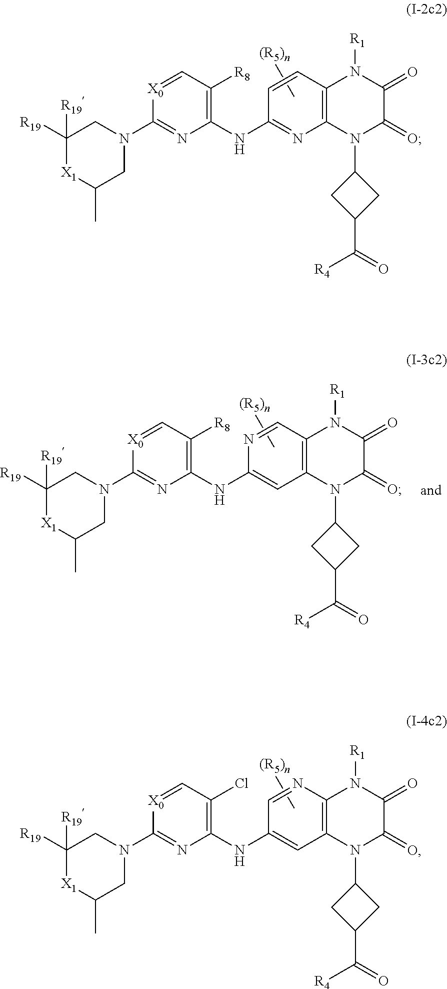
or a pharmaceutically acceptable salt or stereoisomer thereof.

39. The compound of claim 1, wherein 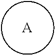
is phenyl, 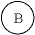
is 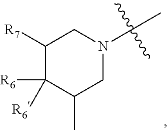
X is SO₂, m is 1 or 2, and o is 0, and the compound is represented by structure I-1d1: 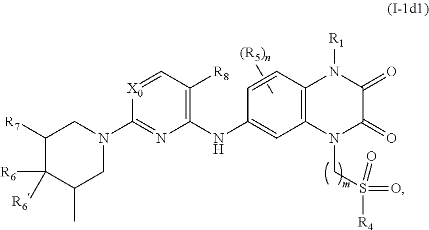
or a pharmaceutically acceptable salt or stereoisomer thereof, or
wherein 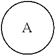
is phenyl, 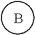
is 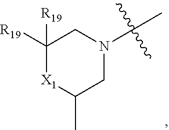
X is SO₂, m is 1 or 2, and o is 0, and the compound is represented by structure I-1d2: 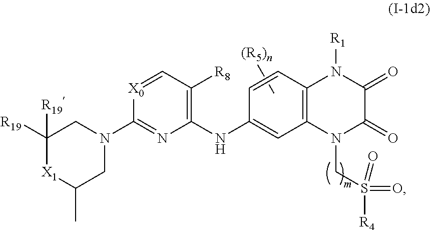
or a pharmaceutically acceptable salt or stereoisomer thereof.

40. (canceled)

41. The compound of claim 1, wherein 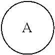
is pyridyl, 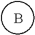
is 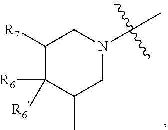
X is SO₂, m is 1 or 2, and o is 0, and the compound is represented by any one of structures I-2d1 to I-4d1: 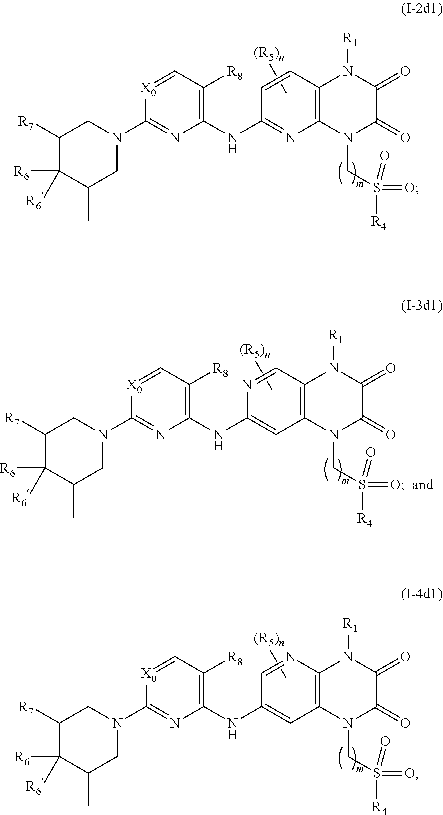
or a pharmaceutically acceptable salt or stereoisomer thereof.

42. The compound of claim 1, wherein 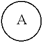
is pyridyl, 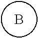
is 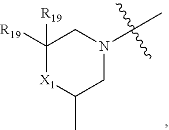
X is SO₂, m is 1 or 2, and o is 0, and the compound is represented by any one of structures I-2d2 to I-4d2: 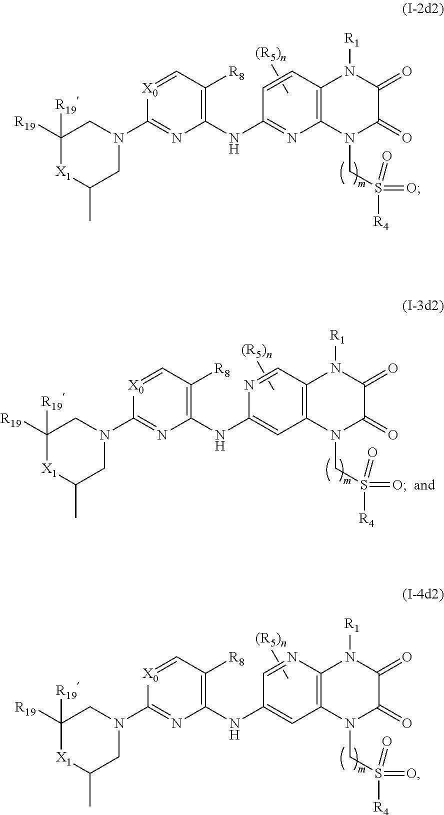
or a pharmaceutically acceptable salt or stereoisomer thereof.

43. The compound of claim 1, wherein R₄ is optionally substituted NH—(C₁-C₆) alkyl, optionally substituted NH—(C₁-C₆) hydroxyalkyl, or optionally substituted NH—(C₁-C₆) aminoalkyl.

44. The compound of claim 43, wherein R₄ is NH-methyl,

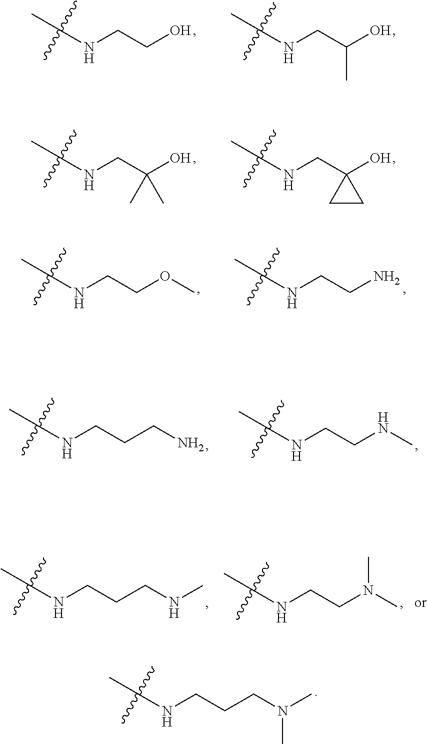

45. The compound of claim 1, wherein R₄ is H or OH.

46.-49. (canceled)

50. The compound of claim 1, wherein R₄ is optionally substituted —NH-4- to 6-membered heterocyclyl or optionally substituted NH—(C₁-C₆) alkyl-4- to 6-membered heterocyclyl.

51. The compound of claim 50, wherein R₄ is

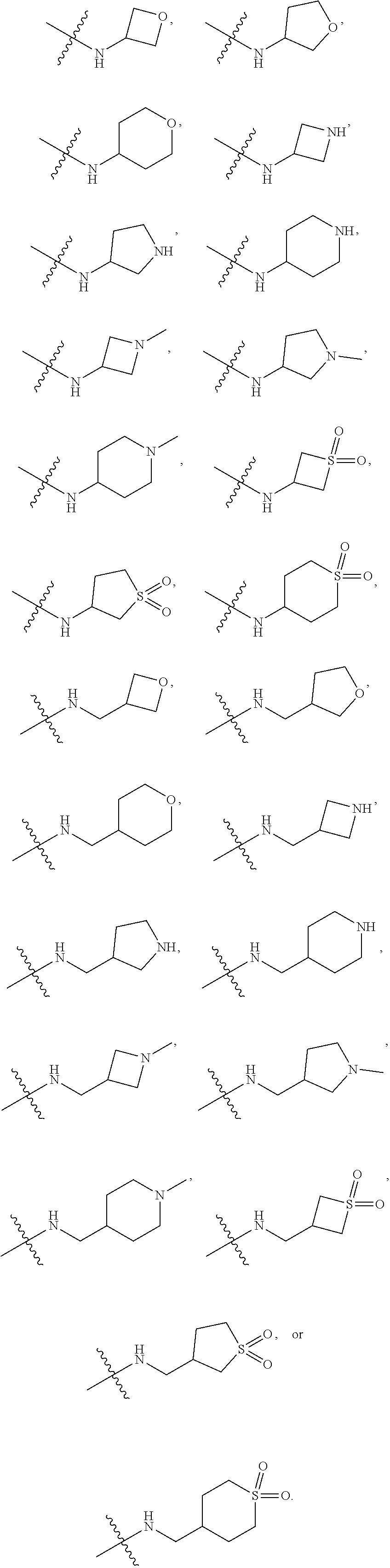

52.-53. (canceled)

54. The compound of claim 1, wherein n is 0 or 1.

55. The compound of claim 1, wherein R₅ is F or methoxy.

56. (canceled)

57. The compound of claim 1, wherein both R₆ and R_{6′} are H or F.

58. The compound of claim 1, wherein X₀ is N.

59. The compound of claim 1, which is any one of the following structures: 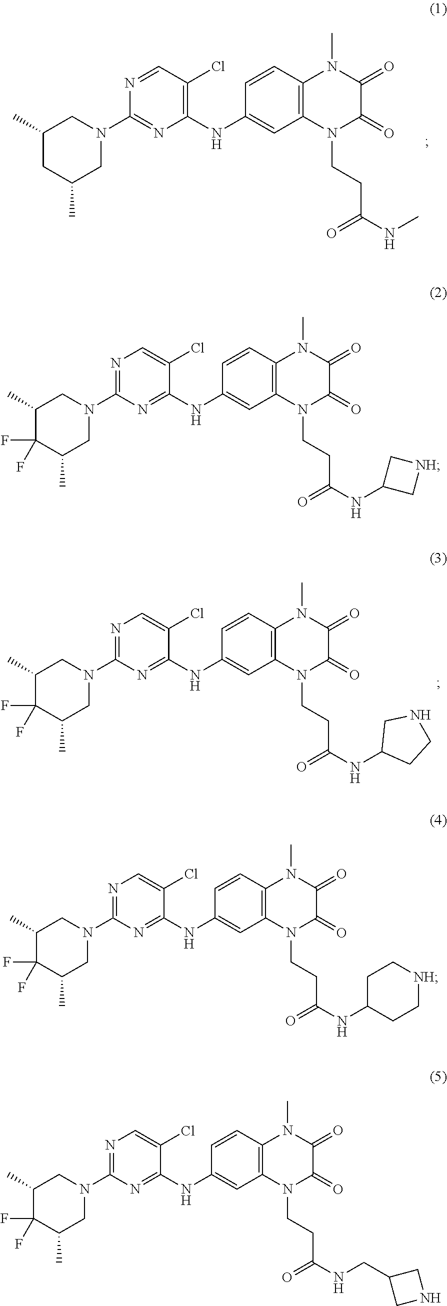 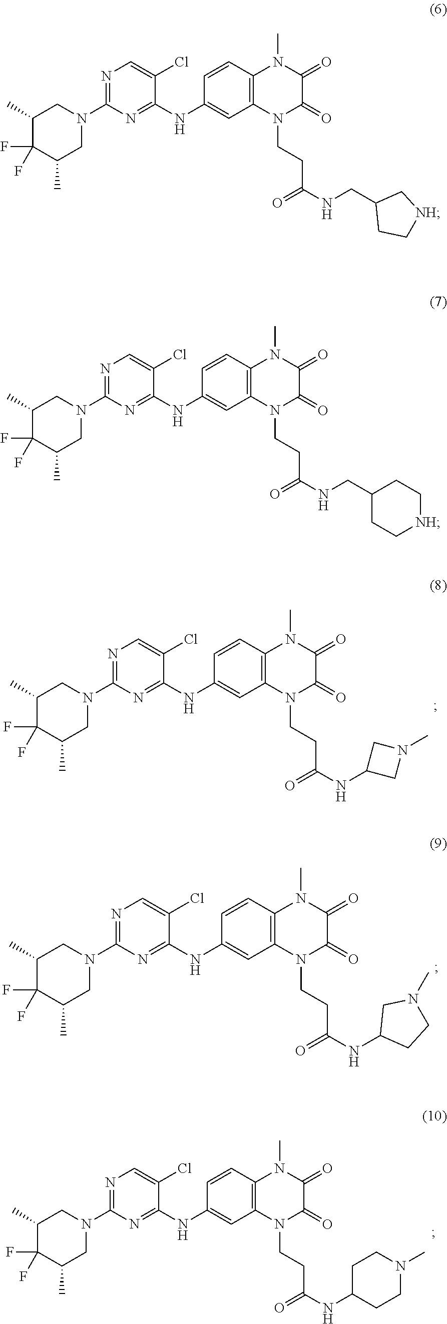 [SMILES: C[C@@H]1CN(c2ncc(Cl)c(Nc3ccc4c(c3)n(CCC(=O)NCC3CCNC3)c(=O)c(=O)n4C)n2)C[C@H](C)C1(F)F ; C[C@@H]1CN(c2ncc(Cl)c(Nc3ccc4c(c3)n(CCC(=O)NCC3CCNCC3)c(=O)c(=O)n4C)n2)C[C@H](C)C1(F)F ; C[C@@H]1CN(c2ncc(Cl)c(Nc3ccc4c(c3)n(CCC(=O)NC3CN(C)C3)c(=O)c(=O)n4C)n2)C[C@H](C)C1(F)F ; C[C@@H]1CN(c2ncc(Cl)c(Nc3ccc4c(c3)n(CCC(=O)NC3CCN(C)C3)c(=O)c(=O)n4C)n2)C[C@H](C)C1(F)F ; C[C@@H]1CN(c2ncc(Cl)c(Nc3ccc4c(c3)n(CCC(=O)NC3CCN(C)CC3)c(=O)c(=O)n4C)n2)C[C@H](C)C1(F)F] 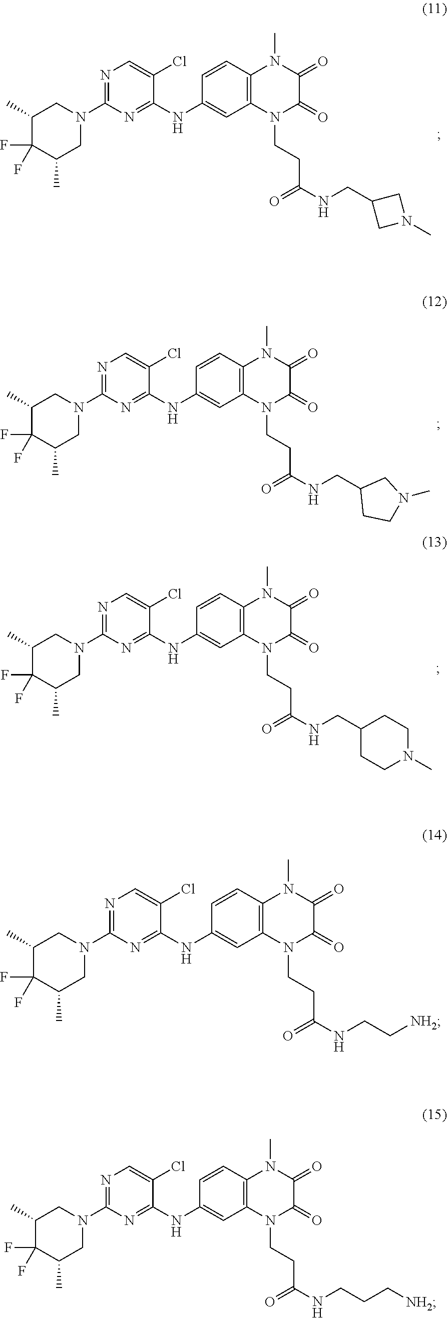 [SMILES: C[C@@H]1CN(c2ncc(Cl)c(Nc3ccc4c(c3)n(CCC(=O)NCC3CN(C)C3)c(=O)c(=O)n4C)n2)C[C@H](C)C1(F)F ; C[C@@H]1CN(c2ncc(Cl)c(Nc3ccc4c(c3)n(CCC(=O)NCC3CCN(C)C3)c(=O)c(=O)n4C)n2)C[C@H](C)C1(F)F ; C[C@@H]1CN(c2ncc(Cl)c(Nc3ccc4c(c3)n(CCC(=O)NCC3CCN(C)CC3)c(=O)c(=O)n4C)n2)C[C@H](C)C1(F)F ; C[C@@H]1CN(c2ncc(Cl)c(Nc3ccc4c(c3)n(CCC(=O)NCCN)c(=O)c(=O)n4C)n2)C[C@H](C)C1(F)F ; C[C@@H]1CN(c2ncc(Cl)c(Nc3ccc4c(c3)n(CCC(=O)NCCCN)c(=O)c(=O)n4C)n2)C[C@H](C)C1(F)F] 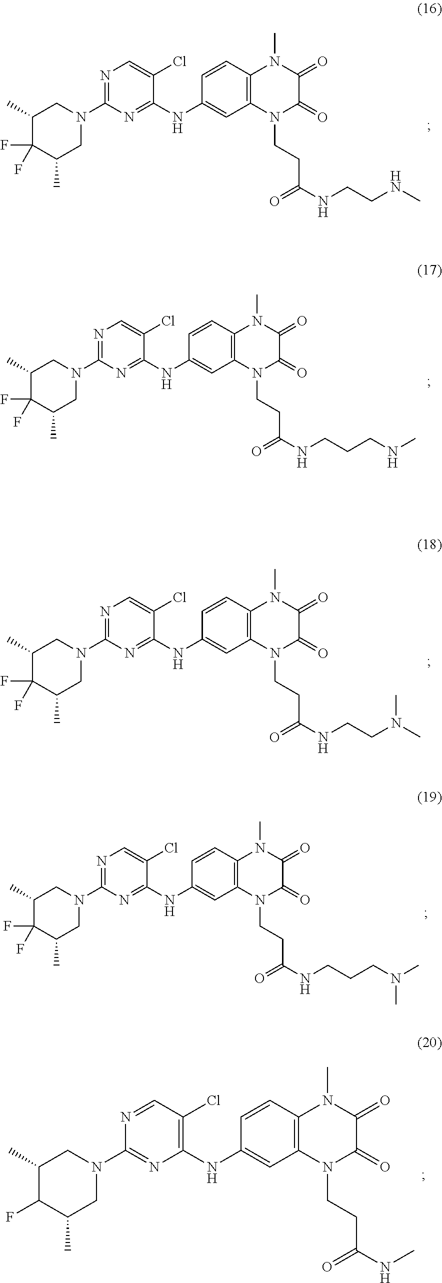 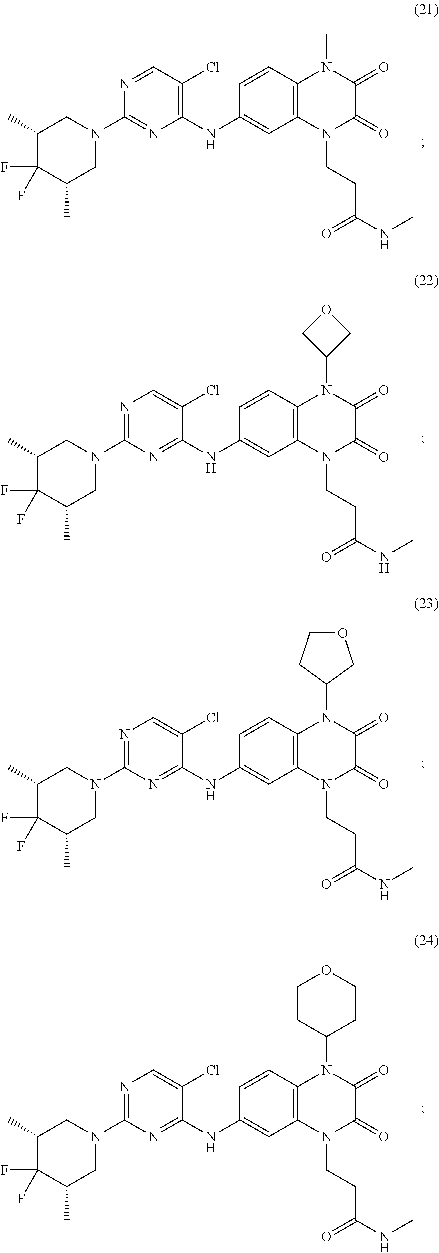 [SMILES: CNC(=O)CCn1c(=O)c(=O)n(C)c2ccc(Nc3nc(N4C[C@@H](C)C(F)(F)[C@@H](C)C4)ncc3Cl)cc21 ; CNC(=O)CCn1c(=O)c(=O)n(C2COC2)c2ccc(Nc3nc(N4C[C@@H](C)C(F)(F)[C@@H](C)C4)ncc3Cl)cc21 ; CNC(=O)CCn1c(=O)c(=O)n(C2CCOC2)c2ccc(Nc3nc(N4C[C@@H](C)C(F)(F)[C@@H](C)C4)ncc3Cl)cc21 ; CNC(=O)CCn1c(=O)c(=O)n(C2CCOCC2)c2ccc(Nc3nc(N4C[C@@H](C)C(F)(F)[C@@H](C)C4)ncc3Cl)cc21] 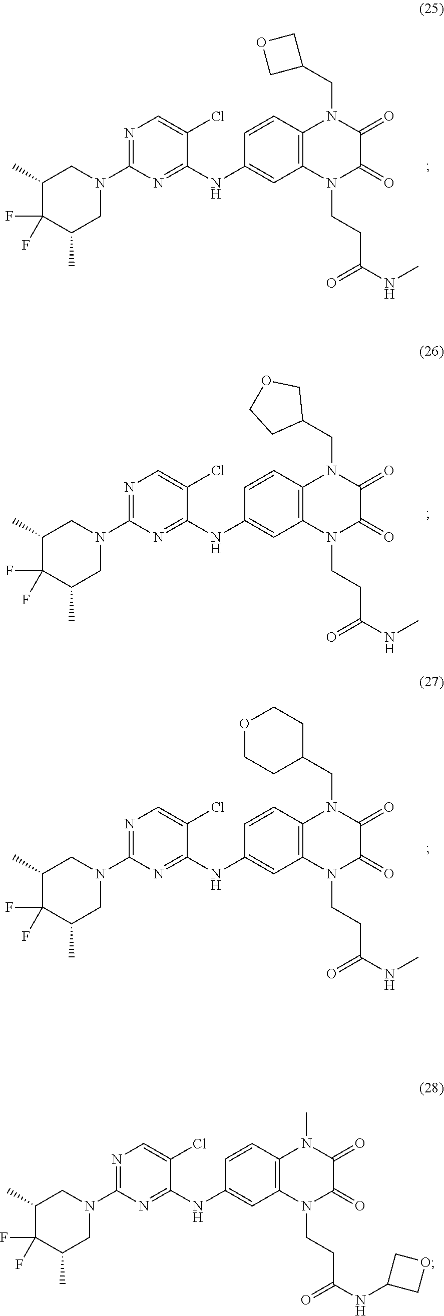 [SMILES: CNC(=O)CCn1c(=O)c(=O)n(CC2COC2)c2ccc(Nc3nc(N4C[C@@H](C)C(F)(F)[C@@H](C)C4)ncc3Cl)cc21 ; CNC(=O)CCn1c(=O)c(=O)n(CC2CCOC2)c2ccc(Nc3nc(N4C[C@@H](C)C(F)(F)[C@@H](C)C4)ncc3Cl)cc21 ; CNC(=O)CCn1c(=O)c(=O)n(CC2CCOCC2)c2ccc(Nc3nc(N4C[C@@H](C)C(F)(F)[C@@H](C)C4)ncc3Cl)cc21 ; C[C@@H]1CN(c2ncc(Cl)c(Nc3ccc4c(c3)n(CCC(=O)NC3COC3)c(=O)c(=O)n4C)n2)C[C@H](C)C1(F)F] 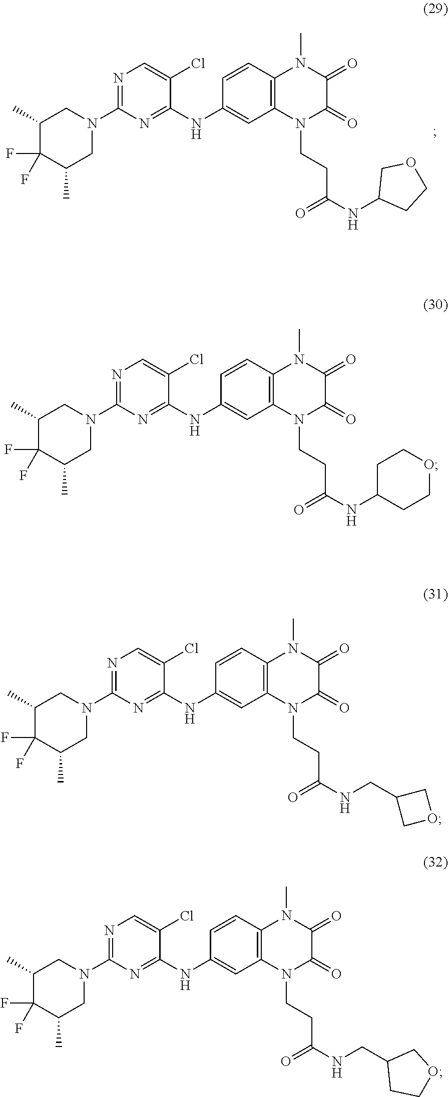 [SMILES: C[C@@H]1CN(c2ncc(Cl)c(Nc3ccc4c(c3)n(CCC(=O)NC3CCOC3)c(=O)c(=O)n4C)n2)C[C@H](C)C1(F)F ; C[C@@H]1CN(c2ncc(Cl)c(Nc3ccc4c(c3)n(CCC(=O)NC3CCOCC3)c(=O)c(=O)n4C)n2)C[C@H](C)C1(F)F ; C[C@@H]1CN(c2ncc(Cl)c(Nc3ccc4c(c3)n(CCC(=O)NCC3COC3)c(=O)c(=O)n4C)n2)C[C@H](C)C1(F)F ; C[C@@H]1CN(c2ncc(Cl)c(Nc3ccc4c(c3)n(CCC(=O)NCC3CCOC3)c(=O)c(=O)n4C)n2)C[C@H](C)C1(F)F] 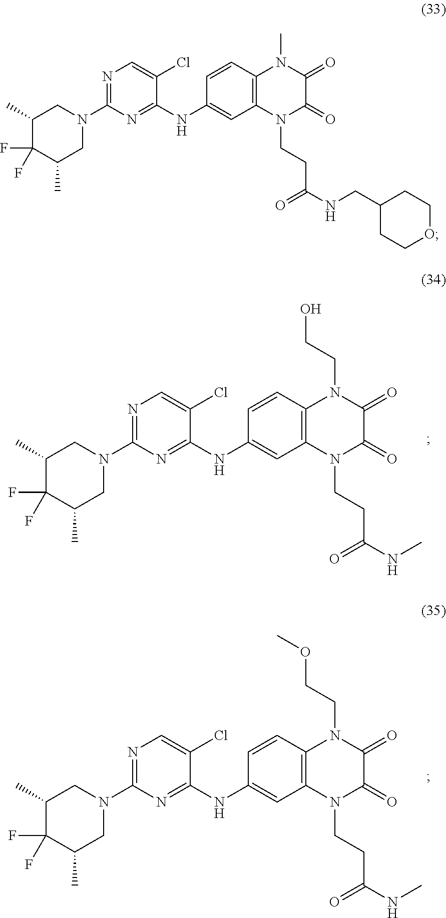 [SMILES: C[C@@H]1CN(c2ncc(Cl)c(Nc3ccc4c(c3)n(CCC(=O)NCC3CCOCC3)c(=O)c(=O)n4C)n2)C[C@H](C)C1(F)F ; CNC(=O)CCn1c(=O)c(=O)n(CCO)c2ccc(Nc3nc(N4C[C@@H](C)C(F)(F)[C@@H](C)C4)ncc3Cl)cc21 ; CNC(=O)CCn1c(=O)c(=O)n(CCOC)c2ccc(Nc3nc(N4C[C@@H](C)C(F)(F)[C@@H](C)C4)ncc3Cl)cc21] 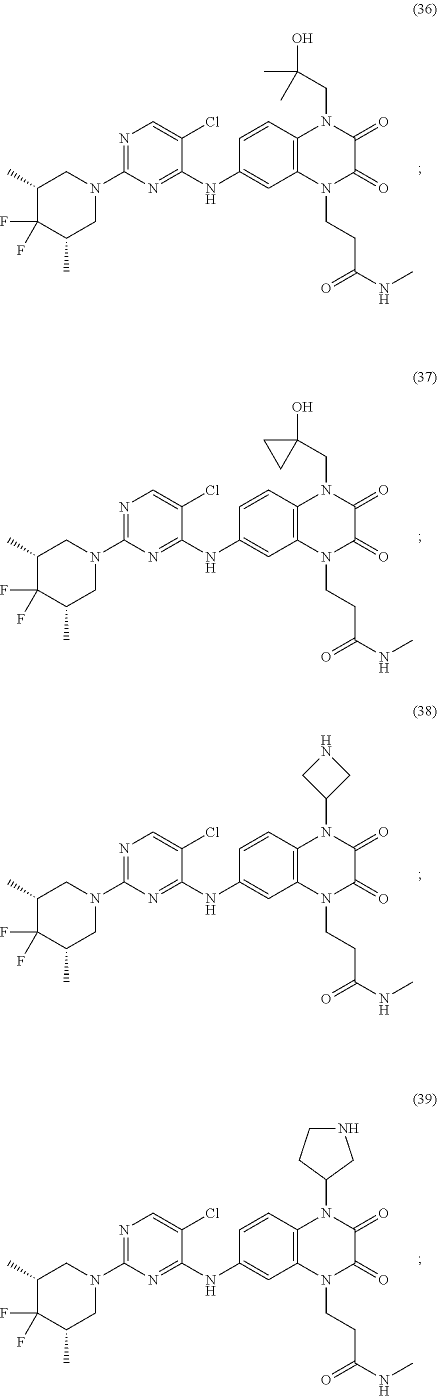 [SMILES: CNC(=O)CCn1c(=O)c(=O)n(CC(C)(C)O)c2ccc(Nc3nc(N4C[C@@H](C)C(F)(F)[C@@H](C)C4)ncc3Cl)cc21 ; CNC(=O)CCn1c(=O)c(=O)n(CC2(O)CC2)c2ccc(Nc3nc(N4C[C@@H](C)C(F)(F)[C@@H](C)C4)ncc3Cl)cc21 ; CNC(=O)CCn1c(=O)c(=O)n(C2CNC2)c2ccc(Nc3nc(N4C[C@@H](C)C(F)(F)[C@@H](C)C4)ncc3Cl)cc21 ; CNC(=O)CCn1c(=O)c(=O)n(C2CCNC2)c2ccc(Nc3nc(N4C[C@@H](C)C(F)(F)[C@@H](C)C4)ncc3Cl)cc21] 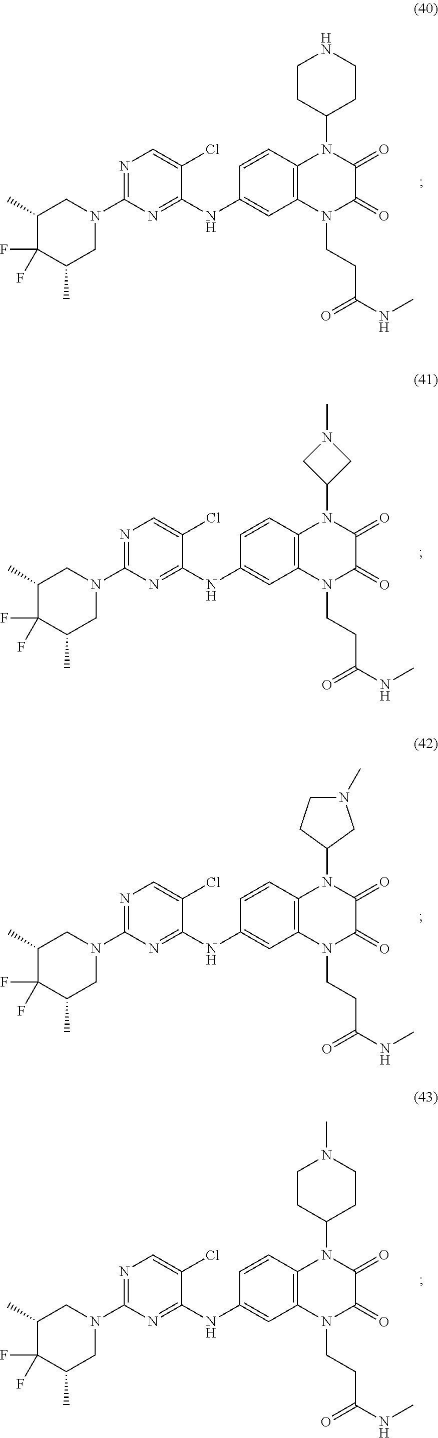 [SMILES: CNC(=O)CCn1c(=O)c(=O)n(C2CCNCC2)c2ccc(Nc3nc(N4C[C@@H](C)C(F)(F)[C@@H](C)C4)ncc3Cl)cc21 ; CNC(=O)CCn1c(=O)c(=O)n(C2CN(C)C2)c2ccc(Nc3nc(N4C[C@@H](C)C(F)(F)[C@@H](C)C4)ncc3Cl)cc21 ; CNC(=O)CCn1c(=O)c(=O)n(C2CCN(C)C2)c2ccc(Nc3nc(N4C[C@@H](C)C(F)(F)[C@@H](C)C4)ncc3Cl)cc21 ; CNC(=O)CCn1c(=O)c(=O)n(C2CCN(C)CC2)c2ccc(Nc3nc(N4C[C@@H](C)C(F)(F)[C@@H](C)C4)ncc3Cl)cc21] 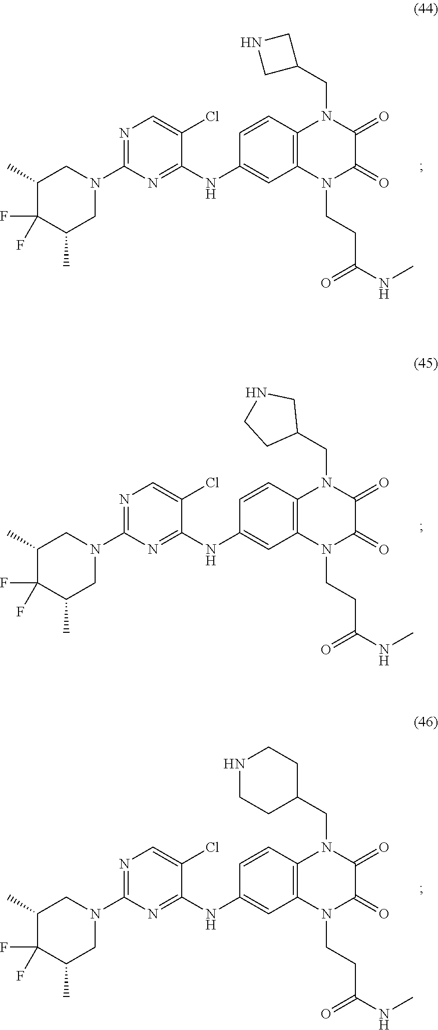 [SMILES: CNC(=O)CCn1c(=O)c(=O)n(CC2CNC2)c2ccc(Nc3nc(N4C[C@@H](C)C(F)(F)[C@@H](C)C4)ncc3Cl)cc21 ; CNC(=O)CCn1c(=O)c(=O)n(CC2CCNC2)c2ccc(Nc3nc(N4C[C@@H](C)C(F)(F)[C@@H](C)C4)ncc3Cl)cc21 ; CNC(=O)CCn1c(=O)c(=O)n(CC2CCNCC2)c2ccc(Nc3nc(N4C[C@@H](C)C(F)(F)[C@@H](C)C4)ncc3Cl)cc21] 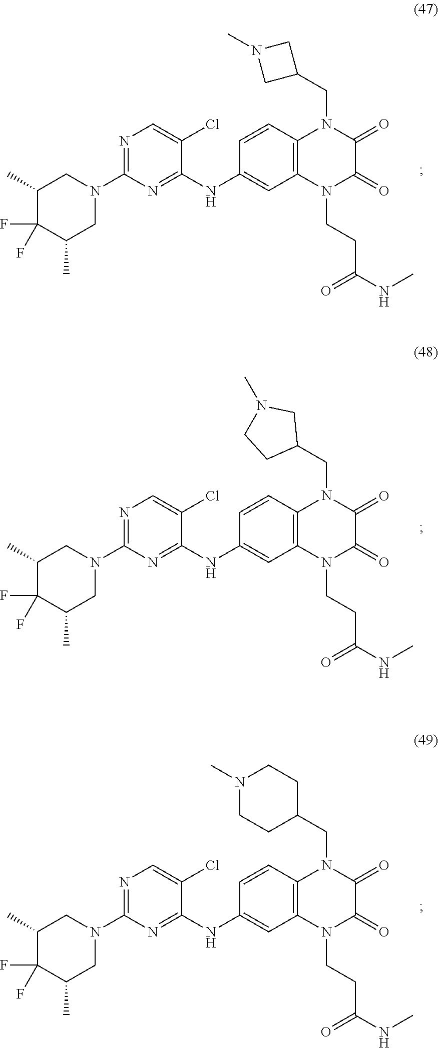 [SMILES: CNC(=O)CCn1c(=O)c(=O)n(CC2CN(C)C2)c2ccc(Nc3nc(N4C[C@@H](C)C(F)(F)[C@@H](C)C4)ncc3Cl)cc21 ; CNC(=O)CCn1c(=O)c(=O)n(CC2CCN(C)C2)c2ccc(Nc3nc(N4C[C@@H](C)C(F)(F)[C@@H](C)C4)ncc3Cl)cc21 ; CNC(=O)CCn1c(=O)c(=O)n(CC2CCN(C)CC2)c2ccc(Nc3nc(N4C[C@@H](C)C(F)(F)[C@@H](C)C4)ncc3Cl)cc21] 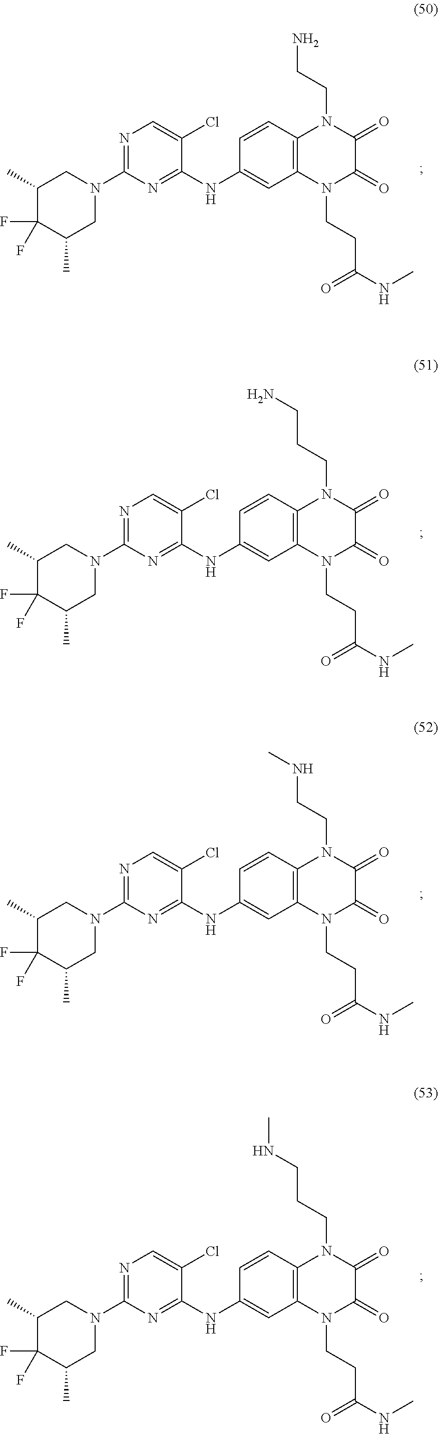 [SMILES: CNC(=O)CCn1c(=O)c(=O)n(CCN)c2ccc(Nc3nc(N4C[C@@H](C)C(F)(F)[C@@H](C)C4)ncc3Cl)cc21 ; CNC(=O)CCn1c(=O)c(=O)n(CCCN)c2ccc(Nc3nc(N4C[C@@H](C)C(F)(F)[C@@H](C)C4)ncc3Cl)cc21 ; CNCCn1c(=O)c(=O)n(CCC(=O)NC)c2cc(Nc3nc(N4C[C@@H](C)C(F)(F)[C@@H](C)C4)ncc3Cl)ccc21 ; CNCCCn1c(=O)c(=O)n(CCC(=O)NC)c2cc(Nc3nc(N4C[C@@H](C)C(F)(F)[C@@H](C)C4)ncc3Cl)ccc21] 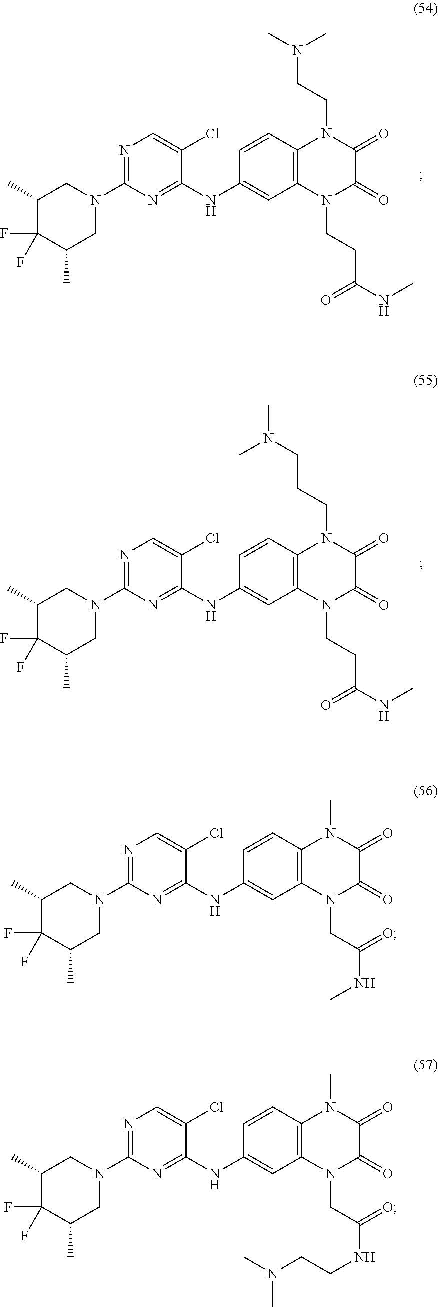 [SMILES: CNC(=O)CCn1c(=O)c(=O)n(CCN(C)C)c2ccc(Nc3nc(N4C[C@@H](C)C(F)(F)[C@@H](C)C4)ncc3Cl)cc21 ; CNC(=O)CCn1c(=O)c(=O)n(CCCN(C)C)c2ccc(Nc3nc(N4C[C@@H](C)C(F)(F)[C@@H](C)C4)ncc3Cl)cc21 ; CNC(=O)Cn1c(=O)c(=O)n(C)c2ccc(Nc3nc(N4C[C@@H](C)C(F)(F)[C@@H](C)C4)ncc3Cl)cc21 ; C[C@@H]1CN(c2ncc(Cl)c(Nc3ccc4c(c3)n(CC(=O)NCCN(C)C)c(=O)c(=O)n4C)n2)C[C@H](C)C1(F)F] 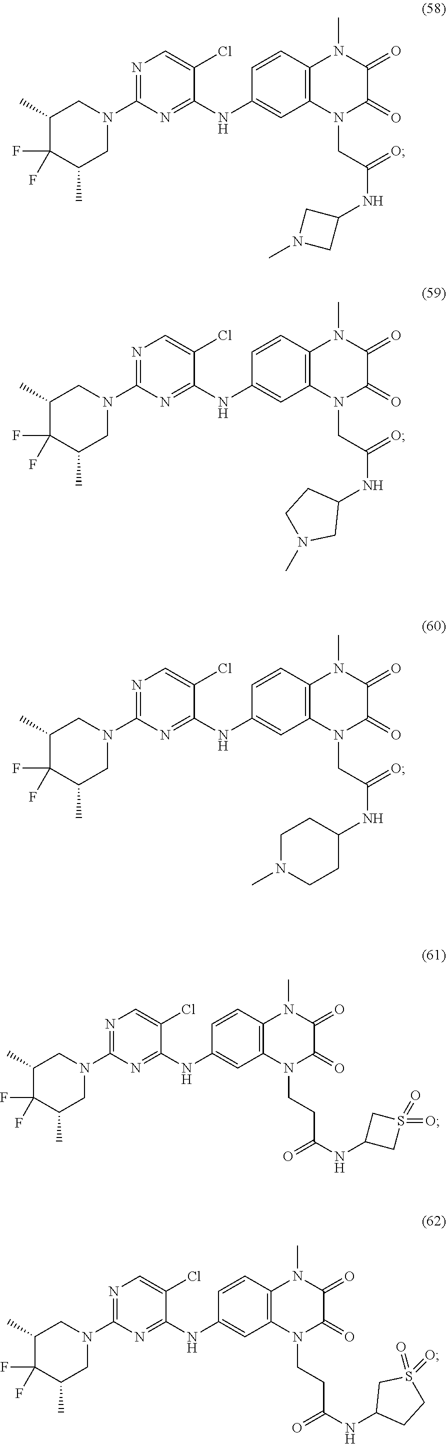 [SMILES: C[C@@H]1CN(c2ncc(Cl)c(Nc3ccc4c(c3)n(CC(=O)NC3CN(C)C3)c(=O)c(=O)n4C)n2)C[C@H](C)C1(F)F ; C[C@@H]1CN(c2ncc(Cl)c(Nc3ccc4c(c3)n(CC(=O)NC3CCN(C)C3)c(=O)c(=O)n4C)n2)C[C@H](C)C1(F)F ; C[C@@H]1CN(c2ncc(Cl)c(Nc3ccc4c(c3)n(CC(=O)NC3CCN(C)CC3)c(=O)c(=O)n4C)n2)C[C@H](C)C1(F)F ; C[C@@H]1CN(c2ncc(Cl)c(Nc3ccc4c(c3)n(CCC(=O)NC3CS(=O)(=O)C3)c(=O)c(=O)n4C)n2)C[C@H](C)C1(F)F ; C[C@@H]1CN(c2ncc(Cl)c(Nc3ccc4c(c3)n(CCC(=O)NC3CCS(=O)(=O)C3)c(=O)c(=O)n4C)n2)C[C@H](C)C1(F)F] 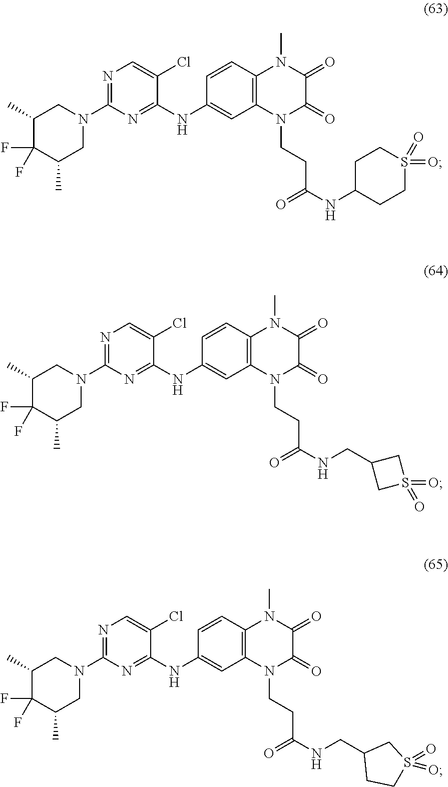 [SMILES: C[C@@H]1CN(c2ncc(Cl)c(Nc3ccc4c(c3)n(CCC(=O)NC3CCS(=O)(=O)CC3)c(=O)c(=O)n4C)n2)C[C@H](C)C1(F)F ; C[C@@H]1CN(c2ncc(Cl)c(Nc3ccc4c(c3)n(CCC(=O)NCC3CS(=O)(=O)C3)c(=O)c(=O)n4C)n2)C[C@H](C)C1(F)F ; C[C@@H]1CN(c2ncc(Cl)c(Nc3ccc4c(c3)n(CCC(=O)NCC3CCS(=O)(=O)C3)c(=O)c(=O)n4C)n2)C[C@H](C)C1(F)F] 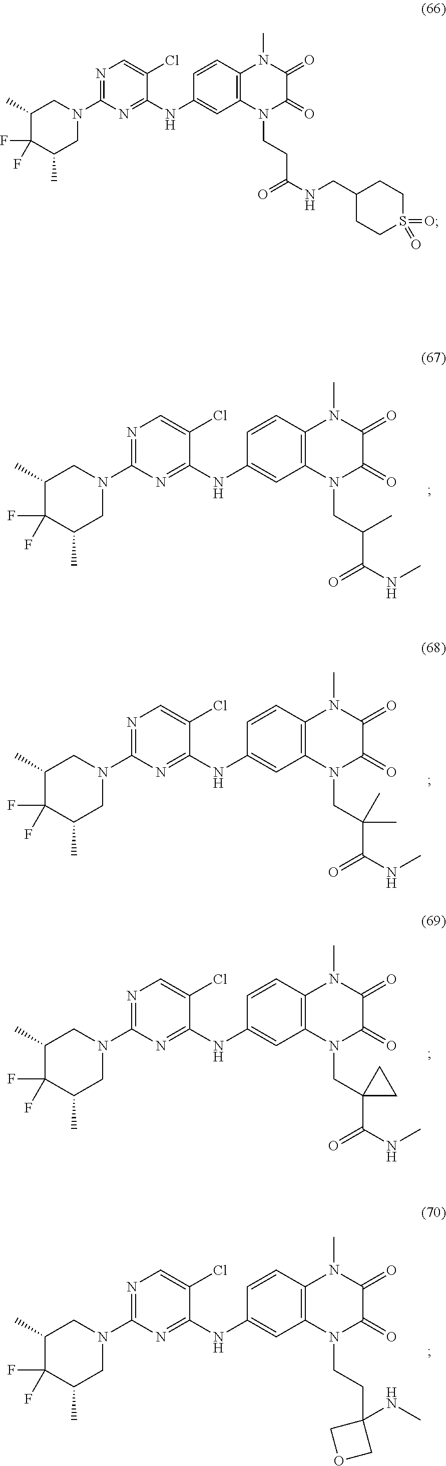 [SMILES: C[C@@H]1CN(c2ncc(Cl)c(Nc3ccc4c(c3)n(CCC(=O)NCC3CCS(=O)(=O)CC3)c(=O)c(=O)n4C)n2)C[C@H](C)C1(F)F ; CNC(=O)C(C)Cn1c(=O)c(=O)n(C)c2ccc(Nc3nc(N4C[C@@H](C)C(F)(F)[C@@H](C)C4)ncc3Cl)cc21 ; CNC(=O)C(C)(C)Cn1c(=O)c(=O)n(C)c2ccc(Nc3nc(N4C[C@@H](C)C(F)(F)[C@@H](C)C4)ncc3Cl)cc21 ; CNC(=O)C1(Cn2c(=O)c(=O)n(C)c3ccc(Nc4nc(N5C[C@@H](C)C(F)(F)[C@@H](C)C5)ncc4Cl)cc32)CC1 ; CNC1(CCn2c(=O)c(=O)n(C)c3ccc(Nc4nc(N5C[C@@H](C)C(F)(F)[C@@H](C)C5)ncc4Cl)cc32)COC1] 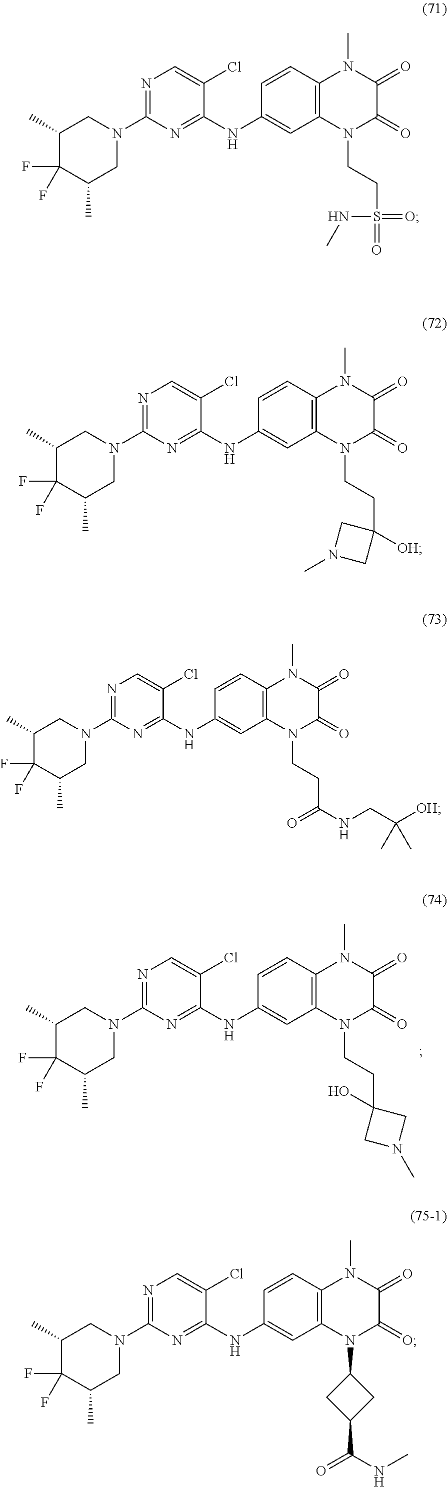 [SMILES: CNS(=O)(=O)CCn1c(=O)c(=O)n(C)c2ccc(Nc3nc(N4C[C@@H](C)C(F)(F)[C@@H](C)C4)ncc3Cl)cc21 ; C[C@@H]1CN(c2ncc(Cl)c(Nc3ccc4c(c3)n(CCC3(O)CN(C)C3)c(=O)c(=O)n4C)n2)C[C@H](C)C1(F)F ; C[C@@H]1CN(c2ncc(Cl)c(Nc3ccc4c(c3)n(CCC(=O)NCC(C)(C)O)c(=O)c(=O)n4C)n2)C[C@H](C)C1(F)F ; C[C@@H]1CN(c2ncc(Cl)c(Nc3ccc4c(c3)n(CCC3(O)CN(C)C3)c(=O)c(=O)n4C)n2)C[C@H](C)C1(F)F ; CNC(=O)[C@H]1C[C@@H](n2c(=O)c(=O)n(C)c3ccc(Nc4nc(N5C[C@@H](C)C(F)(F)[C@@H](C)C5)ncc4Cl)cc32)C1] 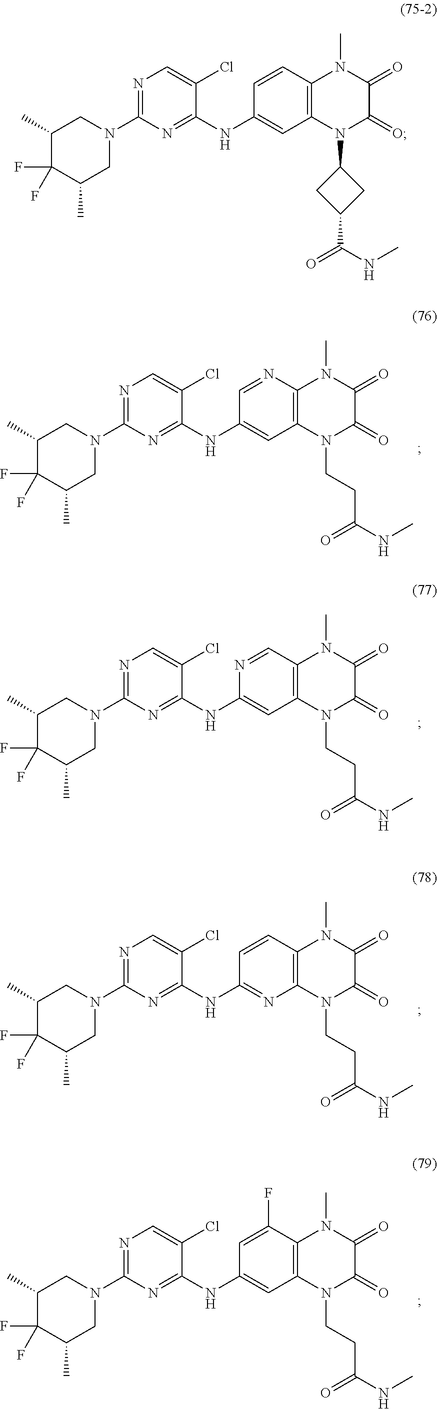 [SMILES: CNC(=O)[C@H]1C[C@H](n2c(=O)c(=O)n(C)c3ccc(Nc4nc(N5C[C@@H](C)C(F)(F)[C@@H](C)C5)ncc4Cl)cc32)C1 ; CNC(=O)CCn1c(=O)c(=O)n(C)c2ncc(Nc3nc(N4C[C@@H](C)C(F)(F)[C@@H](C)C4)ncc3Cl)cc21 ; CNC(=O)CCn1c(=O)c(=O)n(C)c2cnc(Nc3nc(N4C[C@@H](C)C(F)(F)[C@@H](C)C4)ncc3Cl)cc21 ; CNC(=O)CCn1c(=O)c(=O)n(C)c2ccc(Nc3nc(N4C[C@@H](C)C(F)(F)[C@@H](C)C4)ncc3Cl)nc21 ; CNC(=O)CCn1c(=O)c(=O)n(C)c2c(F)cc(Nc3nc(N4C[C@@H](C)C(F)(F)[C@@H](C)C4)ncc3Cl)cc21] 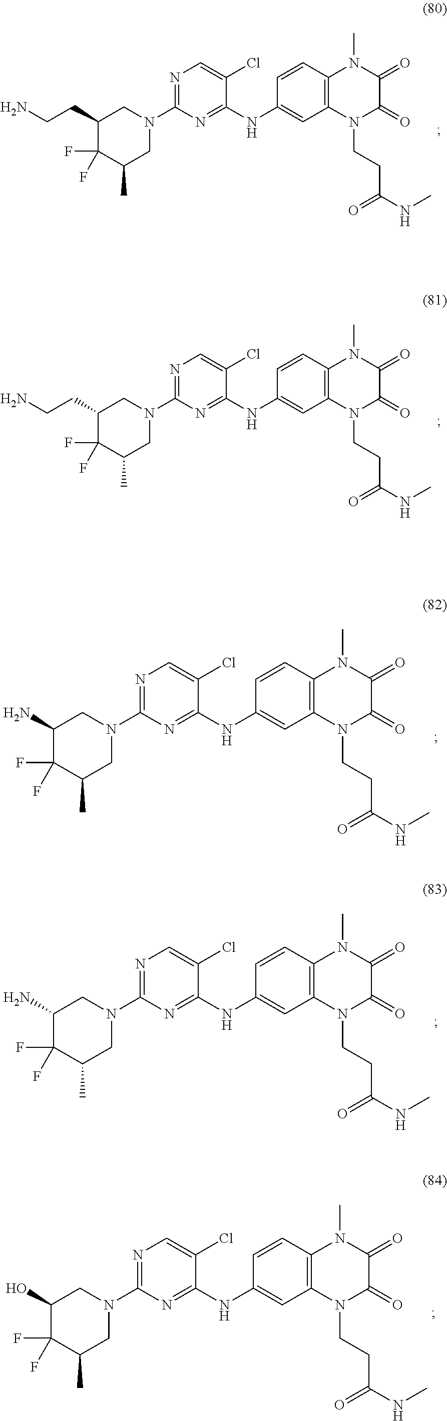 [SMILES: CNC(=O)CCn1c(=O)c(=O)n(C)c2ccc(Nc3nc(N4C[C@H](CCN)C(F)(F)[C@H](C)C4)ncc3Cl)cc21 ; CNC(=O)CCn1c(=O)c(=O)n(C)c2ccc(Nc3nc(N4C[C@@H](CCN)C(F)(F)[C@@H](C)C4)ncc3Cl)cc21 ; CNC(=O)CCn1c(=O)c(=O)n(C)c2ccc(Nc3nc(N4C[C@@H](C)C(F)(F)[C@@H](N)C4)ncc3Cl)cc21 ; CNC(=O)CCn1c(=O)c(=O)n(C)c2ccc(Nc3nc(N4C[C@@H](N)C(F)(F)[C@@H](C)C4)ncc3Cl)cc21 ; CNC(=O)CCn1c(=O)c(=O)n(C)c2ccc(Nc3nc(N4C[C@@H](C)C(F)(F)[C@@H](O)C4)ncc3Cl)cc21] 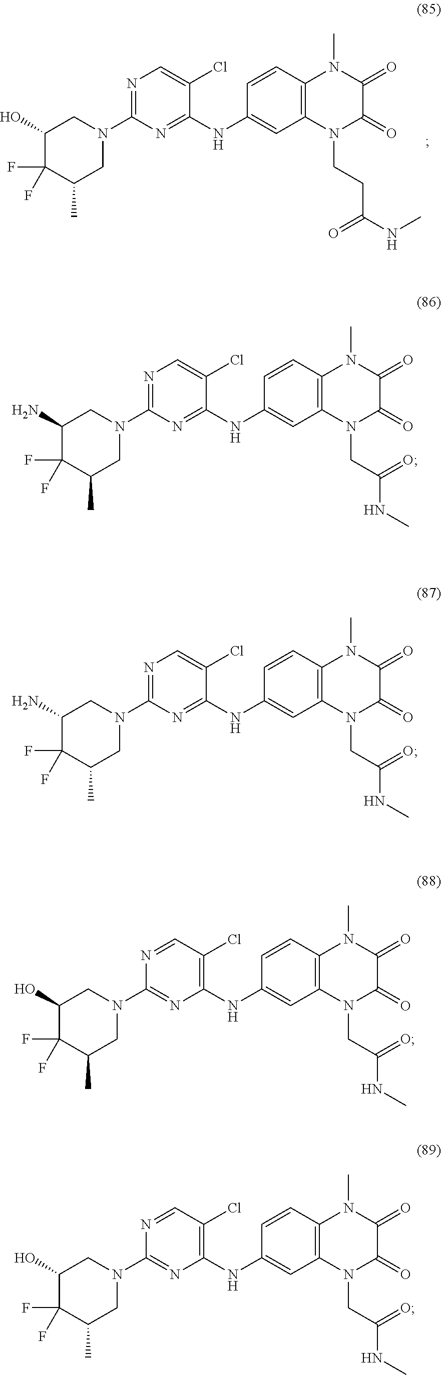 [SMILES: CNC(=O)CCn1c(=O)c(=O)n(C)c2ccc(Nc3nc(N4C[C@@H](O)C(F)(F)[C@@H](C)C4)ncc3Cl)cc21 ; CNC(=O)Cn1c(=O)c(=O)n(C)c2ccc(Nc3nc(N4C[C@@H](C)C(F)(F)[C@@H](N)C4)ncc3Cl)cc21 ; CNC(=O)Cn1c(=O)c(=O)n(C)c2ccc(Nc3nc(N4C[C@@H](N)C(F)(F)[C@@H](C)C4)ncc3Cl)cc21 ; CNC(=O)Cn1c(=O)c(=O)n(C)c2ccc(Nc3nc(N4C[C@@H](C)C(F)(F)[C@@H](O)C4)ncc3Cl)cc21 ; CNC(=O)Cn1c(=O)c(=O)n(C)c2ccc(Nc3nc(N4C[C@@H](O)C(F)(F)[C@@H](C)C4)ncc3Cl)cc21] 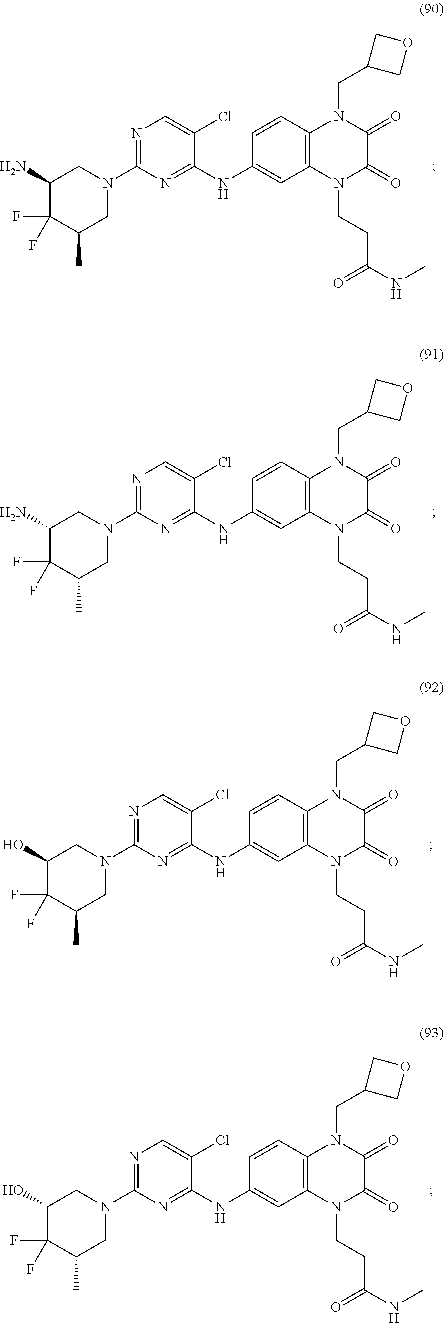 [SMILES: CNC(=O)CCn1c(=O)c(=O)n(CC2COC2)c2ccc(Nc3nc(N4C[C@@H](C)C(F)(F)[C@@H](N)C4)ncc3Cl)cc21 ; CNC(=O)CCn1c(=O)c(=O)n(CC2COC2)c2ccc(Nc3nc(N4C[C@@H](N)C(F)(F)[C@@H](C)C4)ncc3Cl)cc21 ; CNC(=O)CCn1c(=O)c(=O)n(CC2COC2)c2ccc(Nc3nc(N4C[C@@H](C)C(F)(F)[C@@H](O)C4)ncc3Cl)cc21 ; CNC(=O)CCn1c(=O)c(=O)n(CC2COC2)c2ccc(Nc3nc(N4C[C@@H](O)C(F)(F)[C@@H](C)C4)ncc3Cl)cc21] 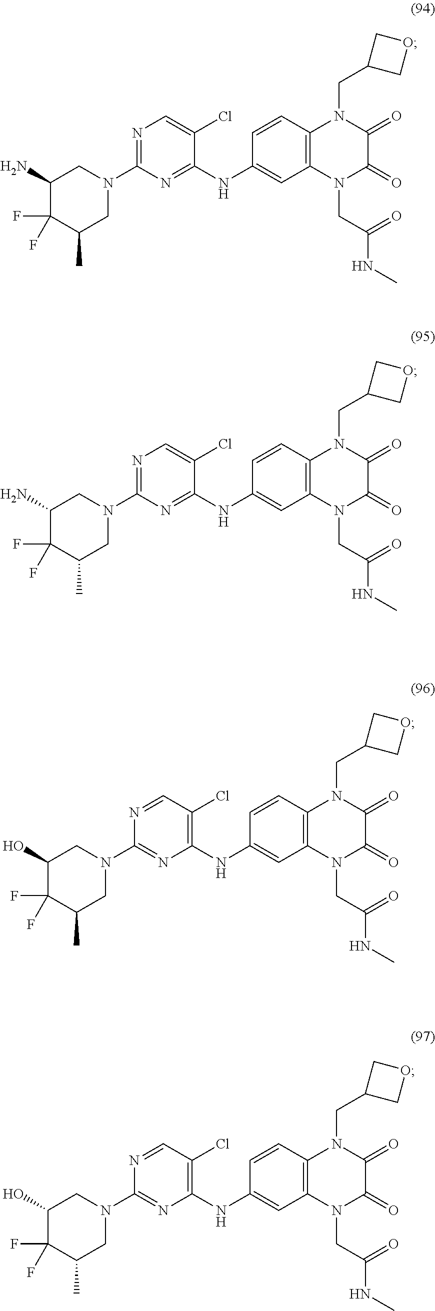 [SMILES: CNC(=O)Cn1c(=O)c(=O)n(CC2COC2)c2ccc(Nc3nc(N4C[C@@H](C)C(F)(F)[C@@H](N)C4)ncc3Cl)cc21 ; CNC(=O)Cn1c(=O)c(=O)n(CC2COC2)c2ccc(Nc3nc(N4C[C@@H](N)C(F)(F)[C@@H](C)C4)ncc3Cl)cc21 ; CNC(=O)Cn1c(=O)c(=O)n(CC2COC2)c2ccc(Nc3nc(N4C[C@@H](C)C(F)(F)[C@@H](O)C4)ncc3Cl)cc21 ; CNC(=O)Cn1c(=O)c(=O)n(CC2COC2)c2ccc(Nc3nc(N4C[C@@H](O)C(F)(F)[C@@H](C)C4)ncc3Cl)cc21] 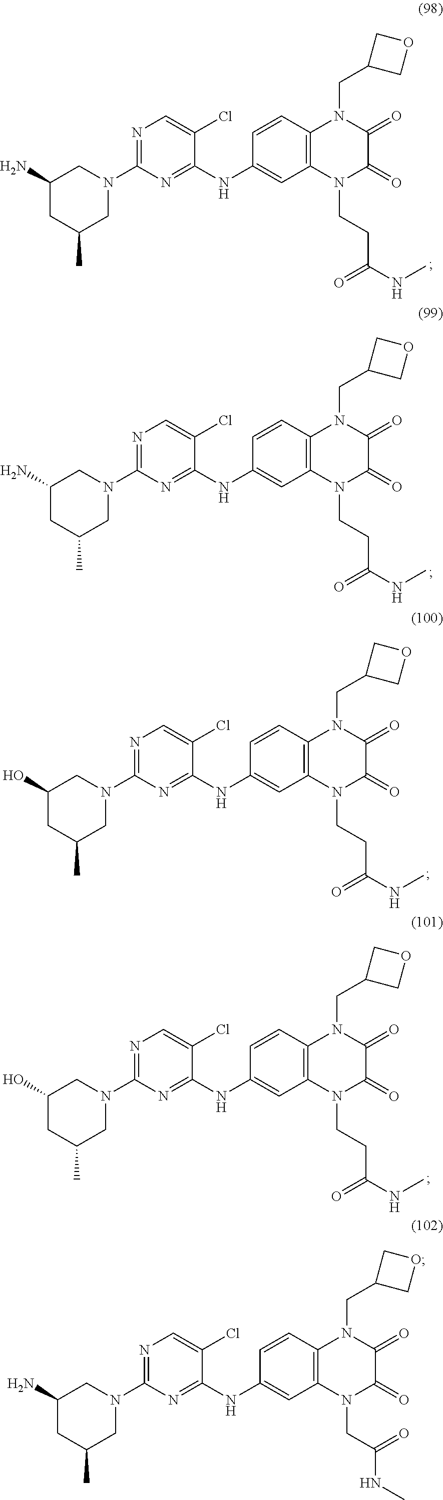 [SMILES: CNC(=O)CCn1c(=O)c(=O)n(CC2COC2)c2ccc(Nc3nc(N4C[C@@H](C)C[C@@H](N)C4)ncc3Cl)cc21 ; CNC(=O)CCn1c(=O)c(=O)n(CC2COC2)c2ccc(Nc3nc(N4C[C@H](C)C[C@H](N)C4)ncc3Cl)cc21 ; CNC(=O)CCn1c(=O)c(=O)n(CC2COC2)c2ccc(Nc3nc(N4C[C@@H](C)C[C@@H](O)C4)ncc3Cl)cc21 ; CNC(=O)CCn1c(=O)c(=O)n(CC2COC2)c2ccc(Nc3nc(N4C[C@H](C)C[C@H](O)C4)ncc3Cl)cc21 ; CNC(=O)Cn1c(=O)c(=O)n(CC2COC2)c2ccc(Nc3nc(N4C[C@@H](C)C[C@@H](N)C4)ncc3Cl)cc21] 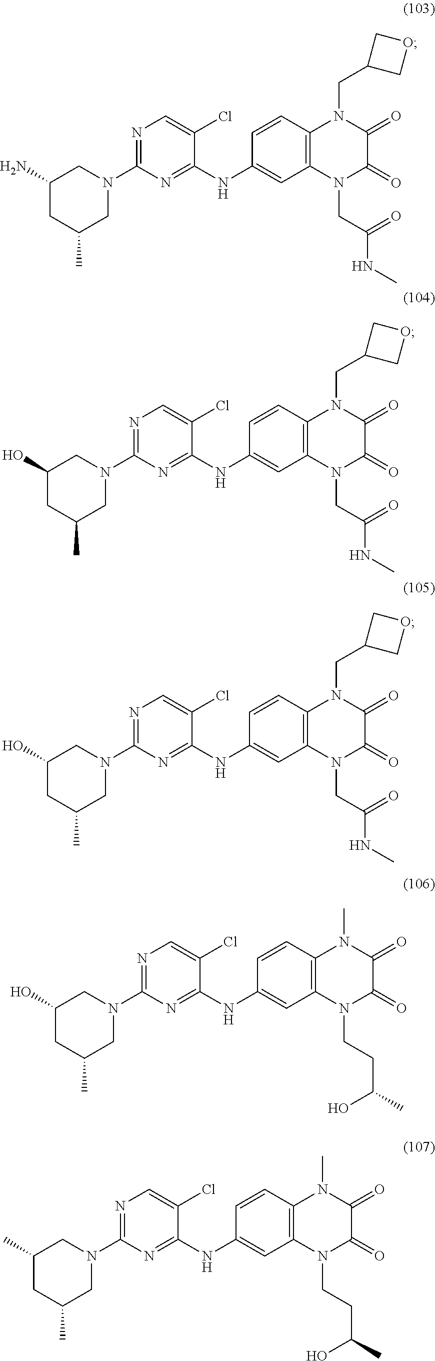 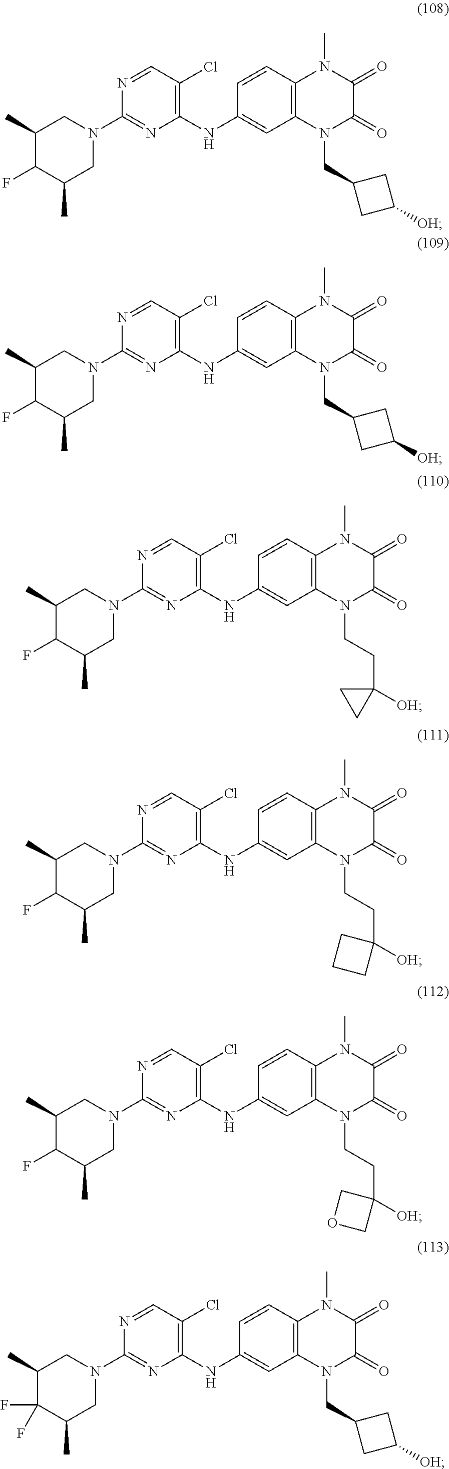 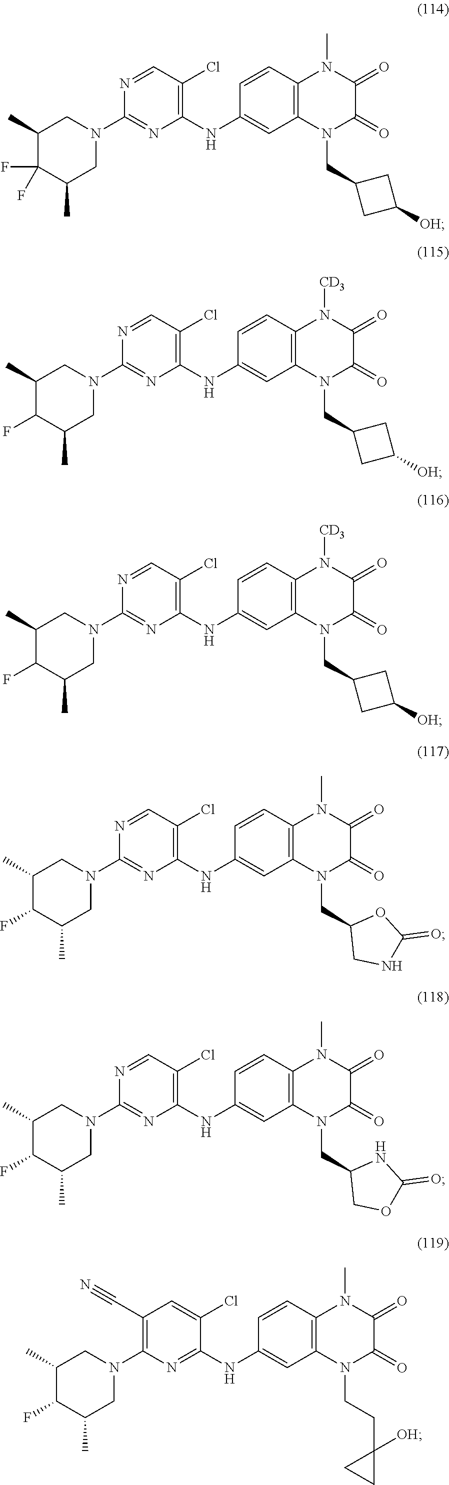 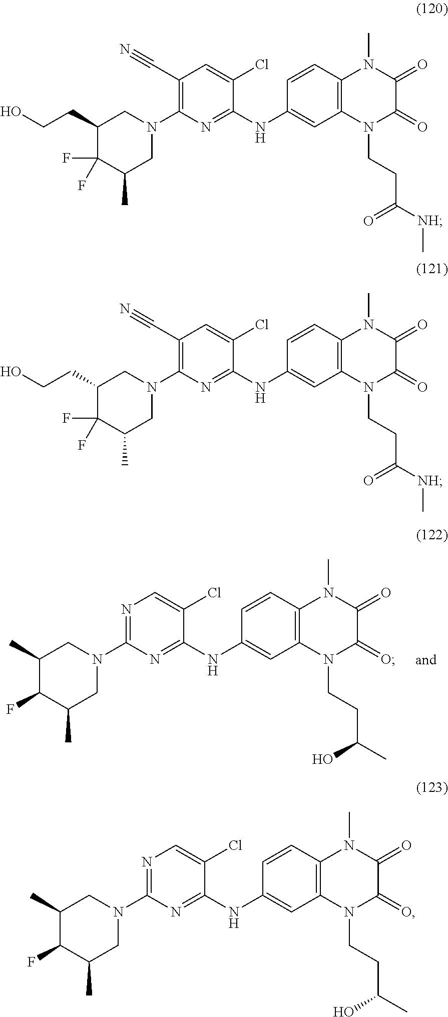
or a pharmaceutically acceptable salt or stereoisomer thereof.

60. A pharmaceutical composition, comprising a therapeutically effective amount of the compound or pharmaceutically acceptable salt or stereoisomer of claim 1, and a pharmaceutically acceptable carrier.

61. The pharmaceutical composition of claim 60, which is in the form of a liquid or a solid.

62. A method of treating a cancer characterized by aberrant B-cell lymphoma 6 (BCL6) activity, comprising administering to a subject in need thereof a therapeutically effective amount of the compound or pharmaceutically acceptable salt or stereoisomer thereof of claim 1.

63. The method of claim 62, wherein the cancer is a lymphoid malignancy.

64. The method of claim 63, wherein the lymphoid malignancy is peripheral T-cell lymphoma (PTCL), diffuse large B-cell lymphoma (DLBCL), mantle cell lymphoma (MCL), follicular lymphoma (FL), chronic lymphocytic leukemia (CLL), acute lymphoblastic leukemia/lymphoma (ALL), or cutaneous T-cell lymphoma.

65. The method of claim 62, further comprising administering an additional anti-cancer agent.

66. The method of claim 65, wherein the additional anti-cancer agent is an enhancer of zeste homolog 2 (EZH2) inhibitor.
